(19) United States
(12) Patent Application Publication
(10) Pub. No.: US 2016/0355835 A1
(43) Pub. Date: Dec. 8, 2016

(54) METHODS OF MODULATING PLANT SEED AND NECTARY CONTENT

(71) Applicant: CARNEGIE INSTITUTION OF WASHINGTON, Washington, DC (US)

(72) Inventor: Wolf B. Frommer, Washington, DC (US)

(73) Assignee: CARNEGIE INSTITUTION OF WASHINGTON, Washington, DC (US)

(21) Appl. No.: 14/774,352

(22) PCT Filed: Mar. 13, 2014
(86) PCT No.: PCT/US14/25310

§ 371 Date: Sep. 10, 2015

Related U.S. Application Data
(60) Provisional application No. 61/779,066, filed on Mar. 13, 2013.

(51) Int. Cl. C12N 15/82

(52) U.S. Cl. CPC C12N 15/8245

(57) ABSTRACT

The present invention relates to methods of increasing the levels of at least one sugar in developing seeds in a plant, with the methods comprising inserting an exogenous nucleic acid, which codes for at least one sugar transporter protein (SWEET protein), into a plant cell to create a transgenic plant cell, and subjecting the transgenic plant cell to conditions that promote expression of the at least one SWEET protein during seed development. The methods results in transgenic plant seeds, and transgenic plants that produce seed, where the levels of at least one sugar are increased as compared to seeds from non-transgenic plants of the same species grown under the same conditions.

### STATEMENT REGARDING FEDERALLY SPONSORED RESEARCH OR DEVELOPMENT

Part of the work performed during development of this invention utilized U.S. Government funds under Department of Energy Grant No. DE-FG02-04ER15542 and National Science Foundation Grant No. 0820730. The U.S. Government has certain rights in this invention.

### SEQUENCE LISTING INFORMATION

A computer readable text file, entitled “056100-5093-WO-SequenceListing.txt,” created on or about 12 Mar. 2014 with a file size of about 923 kb contains the sequence listing for this application and is hereby incorporated by reference in its entirety.

### BACKGROUND OF THE INVENTION

Field of the Invention

The present invention relates to methods of increasing the levels of at least one sugar in developing seeds in a plant, with the methods comprising inserting an exogenous nucleic acid, which codes for at least one sugar transporter protein (SWEET protein), into a plant cell to create a transgenic plant cell, and subjecting the transgenic plant cell to conditions that promote expression of the at least one SWEET protein during seed development. The methods results in transgenic plant seeds, and transgenic plants that produce seed, where the levels of at least one sugar are increased as compared to seeds from non-transgenic plants of the same species grown under the same conditions.

Background of the Invention

Yield potential is determined by the efficiency with which plants intercept light, harness it as chemical energy and ultimately make storage products in harvest organs. Sugars are a dominant currency in these transactions, yet the path from the arrival of sucrose at the terminal phloem endings that enter developing seeds and the subsequent transfer and conversion steps that leads to seed filling are among the least understood parts of the energy conversion chain.

In most plants sucrose is the major form of carbohydrate translocated from source to sink tissues. Sucrose is synthesized predominantly in leaf cells via a pair of enzymes, sucrose phosphate synthase and sucrose phosphate phosphatase, and is then exported into the apoplasm by sucrose transporters of the SWEET family and subsequently imported into the vasculature with the help of sucrose/H⁺ co-transporters of the SUT family. It is assumed that the driving force for sucrose translocation in the phloem is created by active import of sucrose into the veins, thereby creating an osmotic gradient and pressure driven flow and that SWEETs feed the SUTs. One of the least understood areas of carbon allocation is phloem unloading and specifically the transfer of sugars from the maternal phloem to the developing embryo and endosperm. In legumes, post-phloem unloading is assumed to occur symplasmically, via plasmodesmata, followed by efflux of sucrose from the seed coat via an elusive efflux transport mechanism. The developing legume embryo takes up sucrose with the help of sucrose/H⁺ cotransporters of the SUT family. Overexpression of SUT1 in developing embryos of pea led to increased sucrose influx, indicating that there is potential for increasing yield through increasing active influx into the embryo in large seed dicots. Accumulation of carbohydrates in the embryo is further driven by enzymatic conversion of sucrose to hexoses and activated hexoses via invertases and sucrose synthase as well as by consuming these products by synthesis of starch and other storage compounds.

Sucrose-metabolizing enzymes such as cell wall invertase (Mn1) in the basal endosperm transfer layer (BETL) and sucrose synthase (SuSy) in the endosperm also play crucial roles in carbon transfer. This two-step degradation is indicative of re-synthesis of sucrose in the endosperm before conversion into starch. However to date, and despite its pivotal role in determining yield, the path of sugar transfer and metabolism in maize kernels remains somewhat unclear. Little is known about membrane transporters that drive accumulation of sugars in this important organ.

Metabolism and transport are closely coupled at the cellular, subcellular, tissue, and whole organism level. While most modeling of metabolic and transport networks in plant systems have been focused on the cellular level, models at the tissue level that integrate transport and metabolic production, consumption and storage are well established for mammalian systems. Brain, heart and liver models have successfully integrated multiple transported metabolites undergoing metabolism through linked metabolic steps of several pathways in several tissue compartments inside and between cells. Established theoretical frameworks, together with modern computing hardware and software tools, allow numerical solution and testing of models that capture key features of tissue level transport and transformation of substrates and products.

Several published plant studies have integrated transport, metabolism and/or storage to varying degrees. Detailed modeling of spatial and developmental auxin transport and signaling has elegantly illuminated hormonal regulation of meristem growth. Published models of sucrose transport, metabolism and storage in sugarcane led to the identification of control points, and a target for increasing flux to sucrose was identified and experimentally validated by transgenic manipulation.

### SUMMARY OF THE INVENTION

The present invention relates to methods of increasing the levels of at least one sugar in developing seeds in a plant, with the methods comprising inserting an exogenous nucleic acid, which codes for at least one sugar transporter protein (SWEET protein), into a plant cell to create a transgenic plant cell, and subjecting the transgenic plant cell to conditions that promote expression of the at least one SWEET protein during seed development. The methods results in transgenic plant seeds, and transgenic plants that produce seed, where the levels of at least one sugar are increased as compared to seeds from non-transgenic plants of the same species grown under the same conditions.

### BRIEF DESCRIPTION OF THE DRAWINGS

FIG. 1 depicts SWEET9, a sucrose transporter, being necessary for nectar secretion. a-b, Sucrose uptake (a) and efflux (b) activity of AtSWEET9, and BrSWEET9 were performed in Xenopus oocytes. Truncated AtSWEET9_F201* (AtSWEET9m) and BrSWEET9_L201* (BrSWEET9m) served as negative controls. a, Oocyte uptake assay: SWEET9 and SWEET9 mediate ¹⁴C-sucrose uptake (±SEM, n≧14), *t significant at P<0.05., **t significant at P<0.01. b, Oocyte efflux assay: ¹⁴C-sucrose efflux by SWEET9 and SWEET9 in Xenopus oocytes injected with ¹⁴C-sucrose (±SEM, n≧8). c, Nectar droplet clinging to inside of sepal (wild-type). d-e, Lack of nectar in nectaries of sweet9-1 and sweet9-2 mutants. f, Increased nectar secreted from nectaries of flowers containing extra copies of SWEET9-eGFP. g-h, Nectar secreted from nectaries of complemented atsweet9 mutants under its native promoter: SWEET9 (g) or SWEET9-eGFP (h).

FIG. 2 depicts the cellular and subcellular localization of SWEET9 and starch accumulation in sweet9 mutants. a-d, Histochemical GUS analysis in Arabidopsis flowers expressing translational GUS fusion of SWEET9 (native promoter). GUS staining in lateral (a) and median nectaries (b), c-d, Transverse (c) and vertical (d) section of Arabidopsis flowers showing tissue specific localization of SWEET9. Cell walls stained with safranin-O (orange). e, Confocal images of eGFP fluorescence of proSWEET9:SWEET9-eGFP fusion showing subcellular localization at plasma membrane and Golgi. f-g, Flowers of wild-type (f) and sweet9-1 mutant (g) stained with Lugol's iodine solution 4 hours after dawn: starch in the floral stalk of sweet9-1. h-i, Close-up of nectaries for wild-type and sweet9-1. Starch accumulated only in guard cells of wild-type nectaries and in nectary parenchyma in sweet9-1 (sampled at the end of dark). j-k, LR White resin sections of Arabidopsis nectaries in wild-type and sweet9-1 mutants stained with Lugol's iodine solution. Starch grains (dark red) accumulate in nectaries of sweet9-1 mutants (k) and in stomata of wild-type nectary (j, *). Starch grains in floral stalks and nectaries in wild-type and sweet9 mutant lines at anthesis. Cell walls stained with safranin-O (orange).

FIG. 3 depicts that sucrose phosphate synthase 1 (SPS1) and SPS2 are necessary for nectar secretion in Arabidopsis. a-b, Artificial microRNA inhibition of the expression of SPS1 and SPS2 genes lead to a loss of nectar secretion. Arrow indicates the nectar secreted by wild-type flowers. c-d, MicroRNA inhibition of the expression of SPS1 and SPS2 genes altered starch accumulation in the nectaries compared with wild-type. Starch accumulated in the floral stalk of sps1f/2f mutant lines (red arrow) and only in guard cells of wild-type nectaries. e, Proposed model for the mechanism of nectar secretion: starch-derived sucrose is synthesized in nectaries by SPSs and exported by SWEET9. The exported sucrose is subsequently hydrolyzed by CWINV4, which creates a high osmotic potential in order to sustain water flow down the osmotic gradient.

FIG. 4 depicts that SWEET9 in B. rapa (BrSWEET9) and N. attenuata (NaSWEET9) are essential for nectar secretion. a, Nectar droplets in lateral nectary of wild-type B. rapa flowers. b and c, Lack of nectar in brsweet9-1 and brsweet9-2 mutants. d, NaSWEET9 transcript accumulation in N. attenuata. e, Mean (±SEM) nectar volume of flowers measured at 5 am in wild-type, nasweet9-1 and nasweet9-2 plants. f and g, Sucrose uptake (f) and efflux (g) activity of NaSWEET9 in oocytes. Truncated version of NaSWEET9_L201* (NaSWEET9m) served as control. f, Oocyte uptake: NaSWEET9 mediates ¹⁴C-sucrose uptake (±SEM, n≧14), **t significant at P<0.01. g, ¹⁴C-sucrose efflux by NaSWEET9 in oocytes (±SEM, n≧8). h, Data were collected from available genome databases (phytozome.org, genomevolution.org, bioinformatics.psb.ugent.be/plaza/) using SWEET9 protein sequence as bait, while the tree was generated using genomevolution.org as reference, and then confirmed accordingly with results shown in Davies et al. (Proc Natl Acad Sci USA. 2004 Feb. 17, 101(7):1904-9). Tree branches are a schematic representation and they are not defined by any real bootstrap value. Species belonging to the Core Eudicots clades of Rosids or Asterids are underlined in orange and yellow, respectively.

FIG. 5 depicts seed coat expression and mutant phenotype for SWEETs in Arabidopsis. (A) SWEET11-GFP. (B) Starch in wild-type embryos, 8 DAF. (C) A triple mutant of sweet11, 12, 15 (8 DAF) shows retarded development and reduced starch content.

FIG. 6 depicts GFP fusions of SWEET4a, SWEET4b, SWEET4d and SWEET11 localize to the plasma membrane in tobacco. Transient expression in Agrobacterium-infiltrated N. benthamiana leaves demonstrated strong localization of SWEET4a, SWEET4b, SWEET4d and SWEET11 (GFP C-term fusion) at the Plasma Membrane. Fluorescent signals were visualized using confocal laser scanning microscopy, 3 days after Agrobacterium infiltration.

FIG. 7 depicts (A) the function of SWEET4a and SWEET 4b (from maize) as hexose transporters, and (B) the function of SWEET11 (from maize) as a sucrose transporter. Identification of glucose or sucrose transport activity was carried by co-expression with cytosolic FRET glucose or sucrose sensors in HEK293T cells (FLIPglu600μD13V and FLIPsuc90mΔ1V respectively). Individual cells were analyzed by quantitative ratio imaging of CFP and Venus emission (acquisition interval 10s). Co-transfected HEK293T cells were perfused with medium, followed by pulses of 2 mM-5 mM-20 mM glucose or 10 mM Sucrose. HEK293T cells transfected with sensors only (“control”) as controls. SWEET4d is a glucose transporter such as SWEET4a and 4b.

FIG. 8 depicts an insertional allele mutant of SWEET4d in corn. (A) Shows 15DAP kernel phenotype with the wild-type on the left and the mutant on the right: the mutant shows overall reduction in size/weight of about 60% compared to the wild-type kernel. (B) Shows the sagittal cut of wild-type (left) and mutant (right) kernels: both the embryo and the endosperm seem to be heavily affected by the sweet4d mutation, while the maternal pericarp collapses showing an “empty pericarp” phenotype. (C) Shows a corn plant heterozygous for the insertional allele (left) and a homozygous plant (right) for the insertional allele. (D) Schematic drawing of the construct carrying the insertional allele (Mutator) into the last exon. (E) Shows IKI starch staining of the mutant (left) and wild-type (right) corn kernels: in wild-type condition the starch is mostly accumulated within the endosperm and few grains into the root meristem to sustain early germination. In the mutant the endosperm still accumulates starch but its size is dramatically affected, and most of the starch seems to be stored into the embryo.

FIG. 9 depicts the localized expression of SWEETs 11 and 15 in developing seeds of transgenic Arabidopsis carrying native promoter driving SWEET-GFP, respectively.

FIG. 10 depicts the comparison of the embryo phenotype among wild-type (Col), single (sweet15), double (sweet11,12, sweet11,15) and triple mutants (sweet11,12,15) at 8 DAF (Days After Flowering). Embryo of double mutants sweet11,12 and sweet11,15 shows slightly smaller than Col. Triple mutant sweet11,12,15 dramatically delays embryo growth

FIG. 11 depicts the comparison of starch accumulation in embryos of the wild-type (Col), single (sweet15), the triple mutants (sweet11,12,15) and the double mutant (sweet11,12) both at 8 and 11DAF. After siliques were stained for 5 min with Lugol's iodine solution and washed twice with water, embryos were dissected to take pictures. Embryo from triple mutant sweet11,12,15 accumulates less starch than sweet11,12 or Col and embryo from sweet11,12 has more starch than sweet11,12,15, less starch than Col

FIG. 12 depicts a phylogenetic analysis of the 23 Zea mays SWEETs and the 17 SWEET family members from Arabidopsis (At). To further explore the relationship of maize and Arabidopsis SWEETs a phylogenetic tree was constructed (MEGA 5.1) using the closest amino acid sequences from Arabidopsis obtained by a BlastP search of the Phytozome.net non-redundant protein database. The tree demonstrates that also the maize SWEET fall into the SWEET 4 Clades as defined in Arabidopsis.

FIG. 13 depicts amino acid alignment of SWEET4a, 4b and 4d in maize. Asterisks represent the conserved amino acids. Very high homology is observed throughout the all sequences, but decreases drastically within the C-term.

FIG. 14 depicts expression of various SWEETs at various stages of seed development. In this figure, the development pattern follows Arabidopsis SWEET expression. Abbreviation: A, absent; INS, inconsistent detection between biological replicas; M, marginal; P, present. Abbreviation of Stage and Tissue/Compartment: Stage: PGLOB—Pre-Globular Stage; GLOB—Globular Stage; HRT—Heart Stage; LCOT—Linear Cotyledon Stage; MG—Maturation Green Stage. Tissue: CZE—Chalazal Endosperm; CZSC—Chalazal Seed Coat; EP—Embryo Proper; GSC—General Seed Coat; :MCE—Micropylar Endosperm; PEN—Peripheral Endosperm; S—Suspensor; WS—Whole Seed. Signal Intensities (relative mRNA) and signal detection calls (P, A, or M) were generated using MAS 5.0 algorithm. For comparative purposes, GeneChip data were scaled globally to a target intensity of 500 for all probe sets on the chip using MAS 5.0 default parameters. Each probe set was manually assigned a consensus detection call based on the MAS 5.0 detection calls of both biological replicates of an RNA sample. Probe sets with same signal detection calls in both biological replicates were assigned consensus detection calls of P, A, or M, respectively. By contrast, probe sets with different or discordant detection calls for the two biological replicates (e.g., P and A; P and M) were assigned a consensus detection call of Insufficient (INS).

FIG. 15 depicts translational expression of SWEET12 in early seeds development stage. GFP signal was observed in seed coat and suspensor by confocal microscopy.

FIG. 16 depicts translational expression of SWEET15 in different development stages of seeds. SWEET15 localizes to the PM of the outmost layer of seed coat. GFP signal was also visualized in the endosperm at linear cotyledon stage.

FIG. 17 depicts the ability of SWEET11, 12 and 15 to uptake sucrose in oocytes. cRNA of SWEET11, 12 and 15 was injected into oocytes. ¹⁴C-sucrose uptake was measured after 2-day expression.

FIG. 18 depicts Arabidopsis embryo development being delayed in a triple mutant of SWEET 11, 12 and 15. The embryo of triple mutant sweet11,12,15 was mainly arrested from 5 DAF. Images were taken in cleared seeds at different stages by differential interference contrast (DIC) microscopy

FIG. 19 depicts the seed yield of triple mutants of SWEET11, 12 and 15 is lower than that of wild-type Arabidopsis. The sweet11,12 mutant had lower seeds yield than control and higher than sweet11,12,15. Either sweet11,12,15 or sweet11,12 doesn't affect the number of seeds per silique.

FIG. 20 depicts the ability of sucrose to partially rescue root growth of the triple mutant (SWEET11, 12 and 15) when sucrose is added to the growth medium in 5 day-old seedlings.

FIG. 21 depicts the maternal control of seed development being severely impaired in Arabidopsis triple mutant (SWEET11, 12, 15). (A) Two control plants that were crossed show normal seed development at 8DAF. (B) A maternal control was crossed with a paternal triple mutant and the resulting seeds appeared to develop normally at 8DAF. (C) A paternal control was crossed with a maternal triple mutant and the development of the resulting seeds was severely impaired at 8DAF. (D) A paternal triple mutant was crossed with a maternal triple mutant and the development of the resulting seeds was severely impaired at 8DAF. (E) Shows the surface area of the developing seedlings.

FIG. 22 depicts upregulation of SWEET11 in maize mutants in which starch biosynthesis/accumulation is defective. WT-wild-type; ae wx-amylose extender/waxy double mutant; sh1-shruken-1 mutant. Values are on Log scale. Construct in the bottom panel is a schematic representation of the gene SWEET11 and the 2 insertional alleles (DS-ANT and DS-ALV) created by remobilizing an endogenous DS transposon.

FIG. 23 depicts upregulation of SWEET11 in maize in leaves treated 3 days with lanolin and gibberellic acid (GA₃). Young leaves (8 weeks) were spread with a mix of Lanolin and GA₃ for 4 days. Lanolin and GA₃ were then removed to improve RNA extraction. qPCR was carried out using 18S gene as internal standard. Values represent the relative expression of SWEET11 normalized by the internal standard.

FIG. 24 depicts sagittal sections of wild-type and sweet4d mutant phloem termini and BETL. Starch staining was performed leaving the ultrathin (1 m) slides in a saturated IKI solution for 30 min. Black dots are starch grains and they accumulate preferentially within the maternal phloem termini in sweet4d maize mutants.

FIG. 25 depicts aberrant basal endosperm transfer layer (BETL) morphology with no visible cell wall ingrowths or cell organization in SWEET4d maize mutants. Slides were stained with Safranin to highlight cell wall morphology.

FIG. 26 depicts a weblogo of sequence alignment data of Arabidopsis SWEETs showing conserved amino acid sequences. The size of the letter in the weblogo represents the degree of conservation of amino acid sequences among various SWEETs.

FIG. 27 depicts a weblogo of sequence alignment data of Arabidopsis SWEETs showing conserved amino acid sequences. The size of the letter in the weblogo represents the degree of conservation of amino acid sequences among various SWEETs.

### DETAILED DESCRIPTION OF THE INVENTION

The present invention relates to methods of increasing the levels of at least one sugar in developing seeds in a plant, with the methods comprising inserting an exogenous nucleic acid, which codes for at least one sugar transporter protein (SWEET protein), into a plant cell to create a transgenic plant cell, and subjecting the transgenic plant cell to conditions that promote expression of the at least one SWEET protein during seed development. The methods results in transgenic plant seeds, and transgenic plants that produce seed, where the levels of at least one sugar are increased as compared to seeds from non-transgenic plants of the same species grown under the same conditions.

The SWEET proteins, in general, belong to the PFAM family “MtN3_slv” (Accession No. PF03083). See pfam.sanger.ac.uk, which is a database of protein families that are determined and represented by multiple sequence alignments and hidden Markov models (HMMs). In one embodiment of the present invention, the SWEET transporter proteins utilized in the methods, constructs, plants and plant seeds of the present invention are uniporters, which is a well-known term in the art that means a protein that facilitates transport through facilitated diffusion, i.e., the molecules being transported are being transported with the solute gradient. Uniporters do not typically utilize energy for movement of the molecules they transport, other than harnessing the solute gradient.

SWEET proteins are well-known in the art, and their primary amino acid structures can be found in a variety of databases including but not limited to plant membrane protein databases such as aramemnon.botanik.uni-koeln.de, C. elegans protein databases such as www.wormbase.org, and even in human transporter databases, such as www.tcdb.org. In general SWEETs have a characteristic modular structure that is different from other sugar transporters. For example, SWEETs have a different three-dimensional structure from lac permease, yeast hexose transporters, human GLUTs or human SGLTs. The basic unit of a SWEET transporter is a domain composed of three transmembrane domains (TMs). In bacteria, proteins with 3 TMs have to form at least one dimer to create a sugar transporting pore. The eukaryotic versions of the SWEET proteins contain a repeat of this subunit, which is separated by an additional TM domain. This additional TM domain (“TM4”) is not conserved amongst family members, thus the specific amino acid sequence of this domain is not critical to proper functioning across the kingdom of SWEET proteins. This additional TM4 domain serves as an inversion linker that puts the two repeat units of 3 TMs into a parallel configuration, which is how the dimer is formed with the bacterial protein. This 7 TM structure is unique from all other known sugar transporters. That the animal versions of these SWEET proteins as well as bacterial proteins from this same family all transport sugars is indicative that the plant version of these SWEET proteins sugar transporters.

Members of the SWEET transporter superfamily are defined both by conserved amino acid sequences and structural features. For example, all SWEETs are composed of 7 TM divided in two conserved MtN3/saliva motifs embedded in the tandem 3 TM repeat unit, which is connected by a central TM helix that is less conserved, indicating that this central TM serves as a linker. The resulting structure has been described as the 3-1-3 TM SWEET structure.

The first TM domain on average is predicted to be composed of 23 amino acids, but could vary between 20 and 25. Within this TM domain there are at least 4 highly conserved amino acids: G, P, T and F.

The second TM domain on average is predicted to be composed of 19 amino acids, but could vary between 16 and 23. Within this TM domain there are at least 3 highly conserved amino acids: P, Y and Y.

The third TM domain on average is predicted to be composed of 23 amino acids, but could vary between 20 and 25. Within this TM domain there are at least 3 highly conserved amino acids: T, N and G.

The fifth TM domain on average is predicted to be composed of 23 amino acids, but could vary between 20 and 25. Within this TM domain there are at least 3 highly conserved amino acids: G, P and L.

The fifth loop, linking together TM 5 and 6, has 2 highly conserved amino acids: V and T.

The sixth TM domain on average is predicted to be composed of 23 amino acids, but could vary between 19 and 25. Within this TM domain there are at least 7 highly conserved amino acids: S, V, M, P, L, S and Y.

The sixth loop, linking together TM 6 and 7, has a highly conserved amino acid: D.

The seventh TM domain on average is predicted to be composed of 23 amino acids, but could vary between 20 and 25. Within this TM domain there are at least 5 highly conserved amino acids: P, N, G, Q and Y.

Both sugar transport and the seven TM three-dimensional structure are the two key features for this superfamily of proteins. Despite the great variability in size or sequence, and despite the broad number of organisms from which they can be isolated, all SWEETs tested using different heterologous systems have shown sugar transport function.

In one embodiment of the present invention, the SWEET transporter proteins utilized in the methods, constructs, plants and plant seeds of the present invention are sucrose or hexose uniporters. A hexose uniporter is, as the name implies, a transporter protein that transports hexose sugars, e.g., cyclic hexoses, aldohexoses and ketohexoses. Examples of sucrose or hexose uniporters that may be utilized in the methods, constructs, plants and plant seeds of the present invention include but are not limited to glucose uniporters and fructose uniporters.

In general, SWEETs from a particular species of plant can be categorized into clades, or groups, based on amino acid sequence similarity. In maize, for example there are four clades of SWEET proteins based on sequence similarity within each Glade. For example, Clade I in Zea mays contains SWEETS 1a, 1b, 2, 3a and 3b; Clade II contains SWEETs 4a, 4b, 4d, 6a and 6b; Clade III contains SWEETs 11, 12a, 12b, 13a, 13b, 13c, 14a, 14b, 15a and 15b; Clade IV contains SWEETs 16a, 16b and 17. The number of the specific SWEET protein in maize is used to reflect the phylogenetic relationship to Arabidopsis SWEETs, e.g., SWEET11 in maize is most closely related, by sequence comparison, to SWEET 11 in Arabidopsis, and smaller letters are used to indicate a possible gene amplification relative to Arabidopsis.

Accordingly, the numbering of the SWEET proteins, e.g., SWEET 1, SWEET 2, etc., refers to the amino acid sequence of that specific SWEET protein as derived from Arabidopsis thaliana, as well as orthologs in other species, based on amino acid sequence comparison. Thus, although the gene and protein nomenclature refers to genes and proteins identified in The Arabidopsis Information Resource (TAIR) database, which is available on the worldwide web at www.arabidopsis.org, it is understood that the invention is not limited to genes and proteins only in Arabidposis and that the invention encompasses orthologs of genes in other species. For example, it is understood that the methods, constructs, plants and plant seeds of the present invention utilizing the transporter(s) encoded by the genes AtSweet1-At1G21460, AtSweet2-At3G14770, AtSweet3-At5G53190, AtSweet4-At3G28007, AtSweet5-At5G62850, AtSweet6-At1G66770, AtSweet7-At4G10850, AtSweet8-At5G40260, AtSweet9-At2G39060, AtSweet10-At5G50790, AtSweet11-At3G48740, AtSweet12-At5G23660, AtSweet13-At5G50800, AtSweet14-At4G25010, AtSweet15-At5G13170, AtSweet16-At3G16690 and AtSweet17-At4G15920 in Arabidopsis (accession numbers following the gene name, e.g., “At1G21460,” refer accession numbers from the TAIR database, as described above) to can be applied to methods, constructs, plants and plant seeds utilizing the transporter(s) encoded by the orthologous genes in another species. As used herein, orthologous genes are genes from different species that perform the same or similar function and are believed to descend from a common ancestral gene and thus share a certain amount of amino acid identities in their sequence. Often, proteins encoded by orthologous genes have similar or nearly identical amino acid sequence identities to one another, and the orthologous genes themselves have similar nucleotide sequences, particularly when the redundancy of the genetic code is taken into account. Thus, by way of example, the ortholog of a sucrose transporter in Arabidopsis would be a sucrose transporter in another species of plant, regardless of the amino acid sequence of the two proteins.

In specific embodiments, the SWEET transporter proteins used in methods, constructs, plants and plant seeds of the present invention are SWEET proteins from crops plants, such as a food crops, feed crops or biofuels crops. Exemplary important crops may include corn, wheat, soybean, cotton and rice. Crops also include corn, wheat, barley, triticale, soybean, cotton, millet, sorghum, sugarcane, sugar beet, potato, tomato, grapevine, citrus (orange, lemon, grapefruit, etc), lettuce, alfalfa, common bean, fava bean and strawberries, sunflowers and rapeseed, cassava, miscanthus and switchgrass. Other examples of plants include but are not limited to an African daisy, African violet, alfalfa, almond, anemone, apple, apricot, asparagus, avocado, azalea, banana and plantain, beet, bellflower, black walnut, bleeding heart, butterfly flower, cacao, caneberries, canola, carnation, carrot, cassava, diseases, chickpea, cineraria, citrus, coconut palm, coffee, common bean, maize, cotton, crucifers, cucurbit, cyclamen, dahlia, date palm, douglas-fir, elm, English walnut, flax, Acanthaceae, Agavaceae, Araceae, Araliaceae, Araucariacea, Asclepiadaceae, Bignoniaceae, Bromeliaceae, Cactaceae, Commelinaceae, Euphobiaceae, Gentianaceae, Gesneriaceae, Maranthaceae, Moraceae, Palmae, Piperaceae, Polypodiaceae, Urticaceae, Vitaceae, fuchsia, geranium, grape, hazelnut, hemp, holiday cacti, hop, hydrangea, impatiens, Jerusalem cherry, kalanchoe, lettuce, lentil, lisianthus, mango, mimulus, monkey-flower, mint, mustar, oats, papaya, pea, peach and nectarine, peanut, pear, pearl millet, pecan, pepper, Persian violet, pigeonpea, pineapple, pistachio, pocketbook plant, poinsettia, potato, primula, red clover, rhododendron, rice, rose, rye, safflower, sapphire flower, spinach, strawberry, sugarcane, sunflower, sweetgum, sweet potato, sycamore, tea, tobacco, tomato, verbena, and wild rice.

Based on the description of the amino acid sequences of SWEET transporters disclosed herein, one of skill could easily identify any SWEET transporter from virtually any plant species. Once identified, one of skill in the art can use readily available methods for isolating the coding sequence of the identified SWEET protein from a given species to produce nucleic acids encoding the desired SWEET proteins.

In specific embodiments, the SWEET proteins used in methods, constructs, plants and plant seeds of the present invention are SWEET proteins from Zea mays. Examples of nucleic acid sequences and/or amino acid sequences of the SWEET proteins include but are not limited to ZmSweet1a-GRMZM2G039365, ZmSweet1b-GRMZM2G153358, ZmSweet2-GRMZM2G324903, ZmSweet3a-GRMZM2G179679, ZmSweet3b-GRMZM2G060974, ZmSweet4a-GRMZM2G000812, ZmSweet4b-GRMZM2G144581, ZmSweet4d-GRMZM2G137954, ZmSweet6a-GRMZM2G157675, ZmSweet6b-GRMZM2G416965, ZmSweet11-GRMZM2G368827, ZmSweet12a-GRMZM2G133322, ZmSweet12b-GRMZM2G099609, ZmSweet13a-GRMZM2G173669, ZmSweet13b-GRMZM2G021706, ZmSweet13c-GRMZM2G179349, ZmSweet14a-GRMZM2G094955, ZmSweet14b-GRMZM2G015976, ZmSweet15a-GRMZM2G168365, ZmSweet15b-GRMZM5G872392, ZmSweet16a-GRMZM2G106462, ZmSweet16b-GRMZM2G111926, ZmSweet17-GRMZM2G107597. Accession numbers following the gene name, e.g., “GRMZM2G039365,” refer accession numbers from the Maize Genetics and Genomics database at www.maizegdb.org as described above.

In specific embodiments, the SWEET proteins used in methods, constructs, plants and plant seeds of the present invention are SWEET proteins from Orya sativa. Examples of nucleic acid sequences and/or amino acid sequences of the SWEET proteins include but are not limited to OsSweet1a-Os01g65880, OsSweet1b-Os05g35140, OsSweet2a-Os01g36070, OsSweet2b-Os01g50460, OsSweet3a-Os05g12320, OsSweet3b-Os01g12130, OsSweet4-0s02g19820, OsSweet5-0s05g51090, OsSweet6a-Os01g42110, OsSweet6b-Os01g42090, OsSweet7a-Os09g08030, OsSweet7b-Os09g08440, OsSweet7c-Os12g07860, OsSweet7d-Os09g08490, OsSweet7e-Os09g08270, OsSweet11-0s08g42350, OsSweet12-Os03g22590, OsSweet13-Os12g29220, OsSweet14-Os11g31190, OsSweet15-Os02g30910, OsSweet16-Os03g22200. Accession numbers following the gene name, e.g., “Os01g65880,” refer accession numbers from the Greenphyl database (version 4) at www.greenphyl.org as described herein, or the TIGR database at ice.plantbiology.msu.edu.

In specific embodiments, the SWEET proteins used in methods, constructs, plants and plant seeds of the present invention are SWEET proteins from Arabidopsis thaliana. Examples of nucleic acid sequences and/or amino acid sequences of the SWEET proteins include but are not limited to AtSweet1-At1G21460, AtSweet2-At3G14770, AtSweet3-At5G53190, AtSweet4-At3G28007, AtSweet5-At5G62850, AtSweet6-At1G66770, AtSweet7-At4G10850, AtSweet8-At5G40260, AtSweet9-At2G39060, AtSweet10-At5G50790, AtSweet11-At3G48740, AtSweet12-At5G23660, AtSweet13-At5G50800, AtSweet14-At4G25010, AtSweet15-At5G13170, AtSweet16-At3G16690, AtSweet17-At4G15920. Accession numbers following the gene name, e.g., “At5G23660,” refer accession numbers from the TAIR database as described above.

In specific embodiments, the SWEET proteins used in methods, constructs, plants and plant seeds of the present invention are SWEET proteins from Medicago truncatula. Examples of nucleic acid sequences and/or amino acid sequences of the SWEET proteins include but are not limited to MtSWEET2b-AC235677_9, MtSWEET3c-Medtr1g028460, MtSWEET1a-Medtr1g029380, MtSWEET15a-Medtr2g007890, MtSWEET6-Medtr3g080990, MtSWEET1b-Medtr3g089125, MtSWEET3a-Medtr3g090940, MtSWEET3b-Medtr3g090950, MtSWEET13-Medtr3g098910, MtSWEET11-Medtr3g098930, MtSWEET4-Medtr4g106990, MtSWEET15b-Medtr5g067530, MtSWEET9a-Medtr5g092600, MtSWEET5a-Medtr6g007610, MtSWEET5c-Medtr6g007623, MtSWEET5d-Medtr6g007633, MtSWEET5b-Medtr6g007637, MtSWEET2c-Medtr6g034600, MtSWEET9b-Medtr7g007490, MtSWEET15d-Medtr7g405710, MtSWEET15c-Medtr7g405730, MtSWEET2a-Medtr8g042490, MtSWEET14-Medtr8g096310, MtSWEET12-Medtr8g096320, MtSWEET7-Medtr8g099730, MtSWEET16-Mtr.42164.1.S1. Accession numbers following the gene name, e.g., “Medtr1g028460,” refer accession numbers from the legume genome database at www.plantgrn.noble.org as described herein

In specific embodiments, the SWEET proteins used in methods, constructs, plants and plant seeds of the present invention are SWEET proteins from Glycine max. Examples of nucleic acid sequences and/or amino acid sequences of the SWEET proteins include but are not limited to GmSWEET1a-XP003526670, GmSWEET1b-Glyma13g09140, GmSWEET1c-Glyma14g27610, GmSWEET2-XP003540515, GmSWEET3a-XP003544116, GmSWEET3b-Glyma13g08190, GmSWEET3c-ACU24301, GmSWEET3d-Glyma04g41680, GmSWEET4-Glyma17g09840, GmSWEET5a-Glyma19g01280, GmSWEET5b-Glyma19g01270, GmSWEET6a-Glyma20g16160, GmSWEET6b-Glyma13g10560.1, GmSWEET7-Glyma08g02890, GmSWEET9a-XP00355271, GmSWEET9b-XP003552719, GmSWEET9c-Glyma08g48281, GmSWEET10a-XP003532478, GmSWEET10b-Glyma05g38340, GmSWEET10c-NP001237418, GmSWEET10d-XP003523161, GmSWEET10e-Glyma06g17540, GmSWEET11a-XP003532471, GmSWEET11b-Glyma05g38351, GmSWEET12a-Glyma04g37530, GmSWEET12b-XP003526939, GmSWEET15a-Glyma08g19580, GmSWEET15b-Glyma15g05470, GmSWEET15c-XP003524088, GmSWEET15d-XP003551863, GmSWEET15e-Glyma08g47561, GmSWEET15f-Glyma18g53930, GmSWEET16a-Glyma09g04840, GmSWEET16b-Glyma15g16030, GmSWEET17-Glyma19g42040. Accession numbers following the gene name, e.g., “Glyma19g42040,” refer accession numbers from the legume genome database at www.plantgrn.noble.org or the Phytozome database at www.photozome.net, as described herein.

In other embodiments, the methods, constructs, plants and plant seed of the present invention may comprise or comprise the use of at least one exogenous nucleic acid encoding a SWEET protein or variant thereof, wherein the exogenous nucleic acid encodes a SWEET or variant thereof comprising an amino acid sequence that is at least 75%, 80%, 85%, 86%, 87%, 88%, 89%, 90%, 91%, 92%, 93%, 94%, 95%, 96%, 97%, 98% or 99% identical to any one of the amino acid sequences of SEQ ID NOs: 1-410. In another embodiment, the methods, constructs, plants and plant seed of the present invention may comprise or comprise the use of at least one exogenous nucleic acid encoding a SWEET protein or variant thereof, wherein the exogenous nucleic acid encodes a SWEET or variant thereof consists of an amino acid sequence that is at least 75%, 80%, 85%, 86%, 87%, 88%, 89%, 90%, 91%, 92%, 93%, 94%, 95%, 96%, 97%, 98% or 99% identical to any one of the amino acid sequences of SEQ ID NOs: 1-410.

TABLE I
List of Sequences

| Organisms | | Gene name | Gene ID | Function |
|---|---|---|---|---|
| Arabidopsis | 1 | AtSWEET1 | AT1G21460 | sugar |
| thaliana | | | | transport |
| | 2 | AtSWEET2 | A13G14770 | sugar |
| | | | | transport |
| | 2 | AtSWEET3 | AT5G53190 | sugar |
| | | | | transport |
| | 3 | AtSWEET4 | AT3G28007 | sugar |
| | | | | transport |
| | 3 | AtSWEET5 | AT5G62850 | sugar |
| | | | | transport |
| | 4 | AtSWEET6 | AT1G66770 | sugar |
| | | | | transport |
| | 4 | AtSWEET7 | AT4G10850 | sugar |
| | | | | transport |
| | 5 | AtSWEET8 | AT5G40260 | sugar |
| | | | | transport |
| | 5 | AtSWEET9 | AT2G39060 | sugar |
| | | | | transport |
| | 6 | AtSWEET10 | AT5G50790 | sugar |
| | | | | transport |
| | 6 | AtSWEET11 | AT3G48740 | sugar |
| | | | | transport |
| | 7 | AtSWEET12 | AT5G23660 | sugar |
| | | | | transport |
| | 7 | AtSWEET13 | AT5G50800 | sugar |
| | | | | transport |
| | 8 | AtSWEET14 | AT4G25010 | sugar |
| | | | | transport |
| | 8 | AtSWEET15 | AT5G13170 | sugar |
| | | | | transport |
| | 9 | AtSWEET16 | AT3G16690 | sugar |
| | | | | transport |
| | 9 | AtSWEET17 | AT4G15920 | sugar |
| | | | | transport |
| Nicotiana | 10 | NaSWEET9 | NEC1-Q9FPN0 | sugar |
| attenuata | | | | transport |
| Brassica rapa | 10 | BrSWEET9 | AGO61984 | sugar |
| | | | | transport |
| Populus | 11 | PtSWEET10a | Potri.015G101400 | sugar |
| trichocarpa | | | | transport |
| Lotus | 11 | LjSWEET3 | BT145500 | sugar |
| japonicus | | | | transport |
| Oryza sativa | 12 | OsSweet1a | Os01g0881300 | sugar |
| | | | | transport |
| | 13 | OsSweet1b | Os05g0426000 | sugar |
| | | | | transport |
| | 14 | OsSweet2a | Os01g0541800 | sugar |
| | | | | transport |
| | 15 | OsSweet2b | Os01g0700100 | sugar |
| | | | | transport |
| | 16 | OsSweet4 | Os02g0301100 | sugar |
| | | | | transport |
| | 17 | OsSweet11 | Os08g0535200 | sugar |
| | | | | transport |
| | 18 | OsSweet12 | Os03g0347500 | sugar |
| | | | | transport |
| | 19 | OsSweet13 | Os12g0476200 | sugar |
| | | | | transport |
| | 20 | OsSweet14 | Os11g0508600 | sugar |
| | | | | transport |
| Zea mays | 21 | ZmSWEET4a | GRMZM2G000812 | sugar |
| | | | | transport |
| | 22 | ZmSWEET4b | GRMZM2G144581 | sugar |
| | | | | transport |
| | 23 | ZmSWEET4d | GRMZM2G137954 | sugar |
| | | | | transport |
| | 24 | ZmSWEET11 | GRMZM2G368827 | sugar |
| | | | | transport |
| Medicago | 25 | MtSWEET11 | Medtr3g098930 | sugar |
| truncatula | | | | transport |
| Glycine max | 26 | GmSWEET11 | Glyma06g17530.1 | sugar |
| | | | | transport |
| Mus musculus | 27 | MmSWEET1 | MmRAG1_AP1 | sugar |
| | | | | transport |
| Homo sapiens | 28 | HsSWEET1 | HsRAG1_AP1 | sugar |
| | | | | transport |
| Caenorhabditis | 29 | CeSWEET1 | CeK02D7_5 | sugar |
| elegans | | | | transport |
| Xenopus laevis | 30 | XISWEET1 | NP_001084504 | sugar |
| | | | | transport |
| Brady- | 31 | BjSemiSWEET1 | bsr6460 | sugar |
| rhizobium | | | | transport |
| japonicum | | | | |

The invention relates to isolated nucleic acids encoding a SWEET, or variant thereof, and to constructs, cells, host cells, plant tissue and plant seeds comprising these nucleic acids. The nucleic acids of the invention can be DNA or RNA. The nucleic acid molecules can be double-stranded or single-stranded RNA or DNA; single stranded RNA or DNA can be the coding, or sense, strand or the non-coding, or antisense, strand. In particular, the nucleic acids may encode any SWEET or variant thereof, as well as fusion proteins. For example, the nucleic acids of the invention include polynucleotide sequences that encode glutathione-S-transferase (GST) fusion protein, poly-histidine (e.g., His6), poly-HN, poly-lysine, hemagglutinin, HSV-Tag. If desired, the nucleotide sequence of the isolated nucleic acid can include additional non-coding sequences such as non-coding 3′ and 5′ sequences (including regulatory sequences, for example).

The nucleic acid molecules of the invention can be “isolated.” As used herein, an “isolated” nucleic acid molecule or nucleotide sequence is intended to mean a nucleic acid molecule or nucleotide sequence that is not flanked by nucleotide sequences normally flanking the gene or nucleotide sequence (as in genomic sequences) and/or has been completely or partially removed from its native environment, e.g., a cell, tissue. For example, nucleic acid molecules that have been removed or purified from cells are considered isolated. In some instances, the isolated material will form part of a composition, for example, a crude extract containing other substances, buffer system or reagent mix. In other circumstances, the material may be purified to near homogeneity, for example as determined by PAGE or column chromatography such as HPLC. Thus, an isolated nucleic acid molecule or nucleotide sequence can includes a nucleic acid molecule or nucleotide sequence which is synthesized chemically, using recombinant DNA technology or using any other suitable method. To be clear, a nucleic acid contained in a vector would be included in the definition of “isolated” as used herein. Also, isolated nucleotide sequences include recombinant nucleic acid molecules, e.g., DNA, RNA, in heterologous organisms, as well as partially or substantially purified nucleic acids in solution. “Purified,” on the other hand is well understood in the art and generally means that the nucleic acid molecules are substantially free of cellular material, cellular components, chemical precursors or other chemicals beyond, perhaps, buffer or solvent. “Substantially free” is not intended to mean that other components beyond the novel nucleic acid molecules are undetectable. The nucleic acid molecules of the present invention may be isolated or purified. Both in vivo and in vitro RNA transcripts of a DNA molecule of the present invention are also encompassed by “isolated” nucleotide sequences.

The invention also encompasses variations of the nucleotide sequences of the invention, such as those encoding functional fragments or variants of the polypeptides as described herein. Such variants can be naturally-occurring, or non-naturally-occurring, such as those induced by various mutagens and mutagenic processes. Intended variations include, but are not limited to, addition, deletion and substitution of one or more nucleotides which can result in conservative or non-conservative amino acid changes, including additions and deletions.

The invention described herein also relates to fragments of the isolated nucleic acid molecules described herein. The term “fragment” is intended to encompass a portion of a nucleotide sequence described herein which is from at least about 20 contiguous nucleotides to at least about 50 contiguous nucleotides or longer in length. Such fragments may be useful as probes and primers. In particular, primers and probes may selectively hybridize to the nucleic acid molecule encoding the polypeptides described herein. For example, fragments which encode polypeptides that retain activity, as described below, are particularly useful.

The invention also provides nucleic acid molecules that hybridize under high stringency hybridization conditions, such as for selective hybridization, to the nucleotide sequences described herein (e.g., nucleic acid molecules which specifically hybridize to a nucleotide sequence encoding polypeptides described herein and encode a modified growth factor isooherin). Hybridization probes include synthetic oligonucleotides which bind in a base-specific manner to a complementary strand of nucleic acid. Suitable probes include polypeptide nucleic acids, as described in Nielsen et al., Science, 254:1497-1500 (1991).

Such nucleic acid molecules can be detected and/or isolated by specific hybridization e.g., under high stringency conditions. “Stringency conditions” for hybridization is a term of art that refers to the incubation and wash conditions, e.g., conditions of temperature and buffer concentration, which permit hybridization of a particular nucleic acid to a second nucleic acid; the first nucleic acid may be perfectly complementary, i.e., 100%, to the second, or the first and second may share some degree of complementarity, which is less than perfect, e.g., 60%, 75%, 85%, 95% or more. For example, certain high stringency conditions can be used which distinguish perfectly complementary nucleic acids from those of less complementarity.

“High stringency conditions”, “moderate stringency conditions” and “low stringency conditions” for nucleic acid hybridizations are explained in Current Protocols in Molecular Biology, John Wiley & Sons, (1998)), which is incorporated by reference. The exact conditions which determine the stringency of hybridization depend not only on ionic strength, e.g., 0.2×SSC, 0.1×SSC of the wash buffers, temperature, e.g., room temperature, 42° C., 68° C., etc., and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, high, moderate or low stringency conditions may be determined empirically.

By varying hybridization conditions from a level of stringency at which no hybridization occurs to a level at which hybridization is first observed, conditions which will allow a given sequence to hybridize with the most similar sequences in the sample can be determined.

Exemplary conditions are described in Krause, M. H. and S. A. Aaronson, Methods in Enzymology, 200:546-556 (1991), which is incorporated by reference. Washing is the step in which conditions are usually set so as to determine a minimum level of complementarity of the hybrids. Generally, starting from the lowest temperature at which only homologous hybridization occurs, each degree (° C.) by which the final wash temperature is reduced, while holding SSC concentration constant, allows an increase by 1% in the maximum extent of mismatching among the sequences that hybridize. Generally, doubling the concentration of SSC results in an increase in Tm. Using these guidelines, the washing temperature can be determined empirically for high, moderate or low stringency, depending on the level of mismatch sought. Exemplary high stringency conditions include, but are not limited to, hybridization in 50% formamide, 1 M NaCl, 1% SDS at 37° C., and a wash in 0.1×SSC at 60° C. Example of progressively higher stringency conditions include, after hybridization, washing with 0.2×SSC and 0.1% SDS at about room temperature (low stringency conditions); washing with 0.2×SSC, and 0.1% SDS at about 42° C. (moderate stringency conditions); and washing with 0.1×SSC at about 68° C. (high stringency conditions). Washing can be carried out using only one of these conditions, e.g., high stringency conditions, washing may encompass two or more of the stringency conditions in order of increasing stringency. Optimal conditions will vary, depending on the particular hybridization reaction involved, and can be determined empirically.

Equivalent conditions can be determined by varying one or more of the parameters given as an example, as known in the art, while maintaining a similar degree of identity or similarity between the target nucleic acid molecule and the primer or probe used. Hybridizable nucleotide sequences are useful as probes and primers for identification of organisms comprising a nucleic acid of the invention and/or to isolate a nucleic acid of the invention, for example. The term “primer” is used herein as it is in the art and refers to a single-stranded oligonucleotide which acts as a point of initiation of template-directed DNA synthesis under appropriate conditions in an appropriate buffer and at a suitable temperature. The appropriate length of a primer depends on the intended use of the primer, but typically ranges from about 15 to about 30 nucleotides. Short primer molecules generally require cooler temperatures to form sufficiently stable hybrid complexes with the template. A primer need not reflect the exact sequence of the template, but must be sufficiently complementary to hybridize with a template. The term “primer site” refers to the area of the target DNA to which a primer hybridizes. The term “primer pair” refers to a set of primers including a 5′ (upstream) primer that hybridizes with the 5′ end of the DNA sequence to be amplified and a 3′ (downstream) primer that hybridizes with the complement of the 3′ end of the sequence to be amplified.

The nucleic acids described herein can be amplified by methods known in the art. For example, amplification can be accomplished by the polymerase chain reaction (PCR). See PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Eckert et al., PCR Methods and Applications 1:17 (1991); PCR (eds. McPherson et al., IRL Press, Oxford); and U.S. Pat. No. 4,683,202, all of which are incorporated by reference. Other suitable amplification methods include the ligase chain reaction (LCR) (see Wu and Wallace, Genomics, 4:560 (1989), Landegren et al., Science, 241:1077 (1988), both of which are incorporated by reference), transcription amplification (Kwoh et al., Proc. Natl. Acad. Sci. USA, 86:1173 (1989), incorporated by reference), and self-sustained sequence replication (Guatelli et al., Proc. Nat. Acad. Sci. USA, 87:1874 (1990) incorporated by reference) and nucleic acid based sequence amplification (NASBA).

The present invention also relates to vectors that include nucleic acid molecules of the present invention, host cells that are genetically engineered with vectors of the invention and the production of SWEETs or variants thereof by recombinant techniques.

The terms “peptide,” “polypeptide” and “protein” are used interchangeably herein. As used herein, an “isolated polypeptide” is intended to mean a polypeptide that has been completely or partially removed from its native environment. For example, polypeptides that have been removed or purified from cells are considered isolated. In addition, recombinantly produced polypeptides molecules contained in host cells are considered isolated for the purposes of the present invention. Moreover, a peptide that is found in a cell, tissue or matrix in which it is not normally expressed or found is also considered as “isolated” for the purposes of the present invention. Similarly, polypeptides that have been synthesized are considered to be isolated polypeptides. “Purified,” on the other hand is well understood in the art and generally means that the peptides are substantially free of cellular material, cellular components, chemical precursors or other chemicals beyond, perhaps, buffer or solvent. “Substantially free” is not intended to mean that other components beyond the peptides or variants thereof are undetectable.

In specific embodiments, the SWEET proteins used in methods, constructs, plants and plant seeds of the present invention may comprise or comprise the use of a protein or peptide with an amino acid sequence of any one or more of SEQ ID NOs: 1-410.

In other embodiments, the methods, constructs, plants and plant seed of the present invention may comprise or comprise the use of variants of a SWEET protein. In one embodiment, SWEET variants comprise an amino acid sequence that is at least 75%, 80%, 85%, 86%, 87%, 88%, 89%, 90%, 91%, 92%, 93%, 94%, 95%, 96%, 97%, 98% or 99% identical to any one of the amino acid sequences of SEQ ID NOs: 1-410. In another embodiment, the SWEET variants consist of a peptide with an amino acid sequence that is at least 75%, 80%, 85%, 86%, 87%, 88%, 89%, 90%, 91%, 92%, 93%, 94%, 95%, 96%, 97%, 98% or 99% identical to any one of the amino acid sequences of SEQ ID NOs: 1-410.

TABLE II
List of SEQ IDs

| | SEQ |
|---|---|
| Organism | ID NO. |
| Arabidopsis thaliana | |
| AtSweet1-At1G21460-260876_at | 1 |
| AtSweet2-At3G14770-256548_at | 2 |
| AtSweet3-At5G53190-248245_at | 3 |
| AtSweet4-At3G28007-257271_at | 4 |
| AtSweet5-At5G62850-247424_at | 5 |
| AtSweet6-At1G66770-256371_at | 6 |
| AtSweet7-At4G10850-254956_at | 7 |
| AtSweet8-At5G40260-249401_at | 8 |
| AtSweet9-At2G39060-266201_at | 9 |
| AtSweet10-At5G50790-248496_at | 10 |
| AtSweet11-At3G48740-252327_at | 11 |
| AtSweet12-At5G23660-249800_at | 12 |
| AtSweet13-At5G50800-248467_at | 13 |
| AtSweet14-At4G25010-254090_at | 14 |
| AtSweet15-At5G13170-245982_at | 15 |
| AtSweet16-At3G16690-258421_at | 16 |
| AtSweet17-At4G15920-245524_at | 17 |
| Oryza sativa | |
| OsSweet1a-Os01g65880 | 18 |
| OsSweet1b-Os05g35140 | 19 |
| OsSweet2a-Os01g36070 | 20 |
| OsSweet2b-Os01g50460 | 21 |
| OsSweet3a-Os05g12320 | 22 |
| OsSweet3b-Os01g12130 | 23 |
| OsSweet4-Os02g19820 | 24 |
| OsSweet5-Os05g51090 | 25 |
| OsSweet6a-Os01g42110 | 26 |
| OsSweet6b-Os01g42090 | 27 |
| OsSweet7a-Os09g08030 | 28 |
| OsSweet7b-Os09g08440 | 29 |
| OsSweet7c-Os12g07860 | 30 |
| OsSweet7d-Os09g08490 | 31 |
| OsSweet7e-Os09g08270 | 32 |
| OsSweet11-Os08g42350-Os8N3-Os.10401.1.S1_s_at | 33 |
| Os5weet12-Os03g22590 | 34 |
| Os5weet13-Os12g29220-Os12N3 | 35 |
| OsSweet14-Os11g31190-Os11N3 | 36 |
| OsSweet15-Os02g30910 | 37 |
| Os5weet16-Os03g22200 | 38 |
| Zea mays | |
| ZmSweet1a-GRMZM2G039365-Zm.1488.1.S1_at | 39 |
| ZmSweet1b-GRMZM2G153358 | 40 |
| ZmSweet2-GRMZM2G324903-Zm.12522.1.A1_at | 41 |
| ZmSweet3a-GRMZM2G179679-Zm.8559.1.A1_at | 42 |
| ZmSweet3b-GRMZM2G060974 | 43 |
| ZmSweet4a-GRMZM2G000812-Zm.9995.1.A1_at | 44 |
| ZmSweet4b-GRMZM2G144581-Zm.4672.1.S1_at | 45 |
| ZmSweet4d-GRMZM2G137954-Zm.10819.1.S1_at | 46 |
| ZmSweet6a-GRMZM2G157675-Zm.1886.1.S1_at | 47 |
| ZmSweet6b-GRMZM2G416965 | 48 |
| ZmSweet11-GRMZM2G368827-Zm.12529.1.A1_at | 49 |
| ZmSweet12a-GRMZM2G133322 | 50 |
| ZmSweet12b-GRMZM2G099609 | 51 |
| ZmSweet13a-GRMZM2G173669-Zm.1482.3.A1_at | 52 |
| ZmSweet13b-GRMZM2G021706 | 53 |
| ZmSweet13c-GRMZM2G179349 | 54 |
| ZmSweet14a-GRMZM2G094955 | 55 |
| ZmSweet14b-GRMZM2G015976 | 56 |
| ZmSweet15a-GRMZM2G168365-Zm.13688.1.S1_at | 57 |
| ZmSweet15b-GRMZM5G872392-Zm.13688.1.S1_at | 58 |
| ZmSweet16a-GRMZM2G106462-Zm.9036.1.A1_at | 59 |
| ZmSweet16b-GRMZM2G111926 | 60 |
| ZmSweet17-GRMZM2G107597 | 61 |
| Citrus sinensis | |
| CsSweet1-CIT3027 | 62 |
| CsSweet2a-CIT4657 | 63 |
| CsSWEET2b - orange1.1g024679 | 64 |
| CsSWEET3 - orange1.1g042197 | 65 |
| CsSWEET4a - orange1.1g028709 | 66 |
| CsSWEET4b - orange1.1g043313 | 67 |
| CsSWEET5 - orange1.1g037762 | 68 |
| CsSWEET8a - orange1.1g042988 | 69 |
| CsSWEET8b - orange1.1g044881 | 70 |
| CsSweet9-CIT15918 | 71 |
| CsSWEET10 - orange1.1g047365 | 72 |
| CsSWEET11 - orange1.1g036251 | 73 |
| CsSWEET12 - orange1.1g020 | 74 |
| CsSWEET15 - orange1.1g025761 | 75 |
| CsSWEET16a - orange1.1g021755 | 76 |
| CsSWEET16b - orange1.1g039851 | 77 |
| CsSWEET17 - orange1.1g026722 | 78 |
| Medicago truncatula | |
| MtSWEET2b - AC235677_9 | 79 |
| MtSWEET3c - Medtr1g028460 | 80 |
| MtSWEET1a - Medtr1g029380 | 81 |
| MtSWEET15a - Medtr2g007890 | 82 |
| MtSWEET6 - Medtr3g080990 | 83 |
| MtSWEET1b - Medtr3g089125 | 84 |
| MtSWEET3a - Medtr3g090940 | 85 |
| MtSWEET3b - Medtr3g090950 | 86 |
| MtSWEET13 - Medtr3g098910 | 87 |
| MtSWEET11 - Medtr3g098930 | 88 |
| MtSWEET4 - Medtr4g106990 | 89 |
| MtSWEET15b - Medtr5g067530 | 90 |
| MtSWEET9a - Medtr5g092600 | 91 |
| MtSWEET5a - Medtr6g007610 | 92 |
| MtSWEET5c - Medtr6g007623 | 93 |
| MtSWEET5d - Medtr6g007633 | 94 |
| MtSWEET5b - Medtr6g007637 | 95 |
| MtSWEET2c - Medtr6g034600 | 96 |
| MtSWEET9b - Medtr7g007490 | 97 |
| MtSWEET15d - Medtr7g405710 | 98 |
| MtSWEET15c - Medtr7g405730 | 99 |
| MtSWEET2a - Medtr8g042490 | 100 |
| MtSWEET14 - Medtr8g096310 | 101 |
| MtSWEET12 - Medtr8g096320 | 102 |
| MtSWEET7 - Medtr8g099730 | 103 |
| MtSWEET16-Mtr.42164.1.S1_at | 104 |
| Triticum aestivum | |
| TaSWEET2-GR302815-65965389 | 105 |
| TaSweet13-EV254168 | 106 |
| Glycine max | |
| GmSweet1-XP003526670-GmaAffx.76027.1.S1_at | 107 |
| GmSweet2-XP003540515-GmaAffx.1401.1.S1_at | 108 |
| GmSweet3a-XP003544116 | 109 |
| GmSweet3b-255647679-ACU24301--GmaAffx.32284.1.S1_at | 110 |
| GmSweet9a-356499604-XP003552719 | 111 |
| GmSweet9b-GM18G53250 | 112 |
| GmSweet11a-Glyma06g17530.1 | 113 |
| GmSweet11b-XP003523161 | 114 |
| GmSweet11c-XP003532478-Gma.7424.1.S1_at | 115 |
| GmSweet12a-GlycineMaxcDNA-clone:GMFL01-46-E1- | 116 |
| Gma.1705.1.S1_a_at | |
| GmSweet12b-XP003526939 | 117 |
| GmSweet15a-XP003551863 | 118 |
| GmSweet15b-XP003524088 | 119 |
| Populus trichocarpa | |
| PtSWEET1a - Potri.005G187300 | 120 |
| PtSWEET17a - Potri.013G013800 | 121 |
| PtSWEET16c - Potri.013G014500 | 122 |
| PtSWEET17b - Potri.013G013900 | 123 |
| PtSWEET16b - Potri.013G014400 | 124 |
| PtSWEET15a - Potri.003G166800 | 125 |
| PtSWEET6 - Potri.003G143100 | 126 |
| PtSWEET2c - Potri.011G103600 | 127 |
| PtSWEET3a - Potri.015G021500 | 128 |
| PtSWEET10b - Potri.015G101600.1 | 129 |
| PtSWEET5 - Potri.015G074300 | 130 |
| PtSWEET10c - Potri.015G101500 | 131 |
| PtSWEET11 - Potri.015G101700 | 132 |
| PtSWEET1b - Potri.002G072600 | 133 |
| PtSWEET9 - Potri.019G030500-PtpAffx.221634.1.S1_at | 134 |
| PtSWEET10a - Potri.015G101400-PtpAffx.212900.1.S1_at | 135 |
| PtSWEET16a - Potri.005G023900 | 136 |
| PtSWEET16d - Potri.008G220600 | 137 |
| PtSWEET2d - Potri.001G355500 | 138 |
| PtSWEET2b - Potri.001G383000 | 139 |
| PtSWEET2a - Potri.001G383400 | 140 |
| PtSWEET4 - Potri.001G344300 | 141 |
| PtSWEET15b - Potri.001G060900 | 142 |
| PtSWEET10d - Potri.012G103200 | 143 |
| PtSWEET3b - Potri.012G031400 | 144 |
| Vitis vinifera | |
| VvSweet1-XP002265836 | 145 |
| VvSweet2a-XP002285636 | 146 |
| VvSweet2b-XP002269484 | 147 |
| VvSweet3-XP002267886 | 148 |
| VvSweet10\|225456416 | 149 |
| VvSweet9\|225436789 | 150 |
| Brachypodium distachyon | |
| BdSweet1a-XP003564773 | 151 |
| BdSweet1b-XP003568408 | 152 |
| BdSweet3-XP003568735 | 153 |
| Hordeum vulgare | |
| HvSweet1aBAJ94374 | 154 |
| HvSweet1b-BAK08026 | 155 |
| HvSweet13-BAJ85621 | 156 |
| HvSweet14-BAJ94651 | 157 |
| HvSweet15 - Contig8708_at-AK373077 | 158 |
| Sorghum bicolor | |
| SbSweet1\|Sb09g020860 | 159 |
| SbSweet2\|Sb03g032190 | 160 |
| SbSweet3a\|Sb09g006950 | 161 |
| SbSweet3b\|Sb03g001520 | 162 |
| SbSweet4a\|Sb04g012910 | 163 |
| SbSweet4b\|Sb04g015420 | 164 |
| SbSweet4c\|Sb04g012920 | 165 |
| SbSweet5\|Sb09g030270 | 166 |
| SbSweet6a\|Sb03g027260 | 167 |
| SbSweet8\|Sb03g003470 | 168 |
| SbSweet11a\|Sb07g026040 | 169 |
| SbSweet11b\|Sb02g029430 | 170 |
| SbSweet12\|Sb01g035490 | 171 |
| SbSweet13a\|Sb08g013620 | 172 |
| SbSweet13b\|Sb08g013840 | 173 |
| SbSweet13c\|Sb08g014040 | 174 |
| SbSweet14\|Sb05g018110 | 175 |
| SbSweet15\|Sb04g021000 | 176 |
| SbSweet16a\|Sb03g012930 | 177 |
| SbSweet16b\|Sb01g035840 | 178 |
| Picea sitchensis | |
| PsSweet1-ACN40940 | 179 |
| PsSweet3-ABK26022 | 180 |
| PsSweet2-A0E75802 | 181 |
| PsSweet8-ADE76727 | 182 |
| PsSweet16-ABK26262 | 183 |
| PsSweet17-ADE76959 | 184 |
| Physcomitrella patens | |
| PpSweet1a - Pp1s127_127V6.1 | 185 |
| PpSweet1b - Pp1s54_64V6.1 | 186 |
| PpSweet2a - Pp1s240_24V6.1 | 187 |
| PpSweet2b - Pp1s307_21V6.1 | 188 |
| PpSweet4 - Pp1s39_291V6.1 | 189 |
| PpSweet8 | 190 |
| Amborella trichopoda | |
| AmboSweet1 - scaffold00071.29 | 191 |
| AmboSweet2 - scaffold00007.362 | 192 |
| AmboSweet3a - scaffold00021.254 | 193 |
| AmboSweet3b - scaffold00015.111 | 194 |
| AmboSweet6 - scaffold00058.125 | 195 |
| AmboSweet7 - scaffold00058.151 | 196 |
| AmboSweet11 - scaffold00016.169 | 197 |
| AmboSweet16 - scaffold00045.233 | 198 |
| Aquilegia caerulea | |
| AcSweet1 - Aquca_014_00360 | 199 |
| AcSweet17 - Aquca_017_00148 | 200 |
| AcSweet3b - Aquca_012_00215 | 201 |
| AcSweet13 - Aquca_011_00056 | 202 |
| AcSweet12 - Aquca_021_00060 | 203 |
| AcSweet4 - Aquca_037_00238 | 204 |
| AcSweet6 - Aquca_001_00818 | 205 |
| AcSweet2b - Aquca_468_00002 | 206 |
| AcSweet16 - Aquca_003_00698 | 207 |
| AcSweet11 - Aquca_003_00877 | 208 |
| AcSweet5 - Aquca_002_00199 | 209 |
| AcSweet2c - Aquca_055_00129 | 210 |
| AcSweet2a - Aquca_022_00050 | 211 |
| AcSweet3a - Aquca_013_00133 | 212 |
| AcSweet7 - Aquca_025_00283 | 213 |
| Chlamydomonas reinhardtii | |
| CrSweet4 - Cre07.g340700 | 214 |
| CrSweet1 - Cre06.g271800 | 215 |
| CrSweet2 - Cre06.g27180 | 216 |
| CrSweet3 - Cre06.g275000 | 217 |
| CrSweet5 - Cre10.g421650 | 218 |
| Lotus japonicus | |
| LjSweet3 | 219 |
| Saccharum officinarum | |
| SCCCLB1004H11.g | 220 |
| SCCCRT2002F04.g | 221 |
| SCJFRZ2015H09.g | 222 |
| SCQGST1029B12.g | 223 |
| SCJLRZ1021E01.g | 224 |
| SCSBFL4070E03.g | 225 |
| SCUTST3085E04.g | 226 |
| SCSGRT2065C08.g | 227 |
| SCEQLB1063D10.g | 228 |
| SCEQRT1031C11.g | 229 |
| SCCCLR1072D05.g | 230 |
| SCJLHR1025D07.g | 231 |
| SCEZSD1079C10.g | 232 |
| Musa acuminata | |
| GSMUA_AchrUn_T01040 | 233 |
| GSMUA_Achr11P09020_001 | 234 |
| GSMUA_Achr5P01260_001 | 235 |
| GSMUA_Achr4P09090_001 | 236 |
| GSMUA_Achr8P25010_001 | 237 |
| GSMUA_Achr1P12680_001 | 238 |
| GSMUA_Achr10P22330_001 | 239 |
| GSMUA_Achr1P25290_001 | 240 |
| GSMUA_Achr8P00620_001 | 241 |
| GSMUA_Achr7P14690_001 | 242 |
| GSMUA_Achr6P09180_001 | 243 |
| GSMUA_AchrUn_randomP01040_001 | 244 |
| GSMUA_Achr10P20300_001 | 245 |
| GSMUA_Achr8P09230_001 | 246 |
| GSMUA_Achr3P32170_001 | 247 |
| GSMUA_Achr6P23850_001 | 248 |
| GSMUA_Achr11P05500_001 | 249 |
| GSMUA_Achr10P11880_001 | 250 |
| GSMUA_Achr3P18700_001 | 251 |
| GSMUA_Achr6P07950_001 | 252 |
| GSMUA_Achr8P10260_001 | 253 |
| GSMUA_Achr11P15500_001 | 254 |
| GSMUA_Achr3P08960_001 | 255 |
| GSMUA_Achr3P08120_001 | 256 |
| GSMUA_Achr9P17640_001 | 257 |
| GSMUA_AchrUn_randomP01030_001 | 258 |
| GSMUA_Achr6P09170_001 | 259 |
| Manioth esculenta | |
| MeSWEET1a - cassava4.1_014638m | 260 |
| MeSWEET1b - cassava4.1_014650m | 261 |
| MeSWEEET2a - cassava4.1_015227m | 262 |
| MeSWEET2b - cassava4.1_030719m | 263 |
| MeSWEET3a - cassava4.1_026477m | 264 |
| MeSWEET3b - cassava4.1_022559m | 265 |
| MeSWEET4 - cassava4.1_016815m | 266 |
| MeSWEET5 - cassava4.1_026390m | 267 |
| MeSWEET6 - cassava4.1_014231m | 268 |
| MeSWEET7 - cassava4.1_028141m | 269 |
| MeSWEET8a - cassava4.1_032999m | 270 |
| MeSWEET8b - cassava4.1_012690m | 271 |
| MeSWEET8c - cassava4.1_014587m | 272 |
| MeSWEET9 - cassava4.1_032222m | 273 |
| MeSWEET10a - cassava4.1_013474m | 274 |
| MeSWEET10b - cassava4.1_015602m | 275 |
| MeSWEET10c - cassava4.1_021350m | 276 |
| MeSWEET10d - cassava4.1_013519m | 277 |
| MeSWEET10e - cassava4.1_032927m | 278 |
| MeSWEET11 - cassava4.1_028116m | 279 |
| MeSWEET12a - cassava4.1_017557m | 280 |
| MeSWEET12b - cassava4.1_018003m | 281 |
| MeSWEET13cassava4.1_026944m | 282 |
| MeSWEET15a - cassava4.1_026251m | 283 |
| MeSWEET15b - cassava4.1_014124m | 284 |
| MeSWEET16a - cassava4.1_014996m | 285 |
| MeSWEET16b - cassava4.1_015143m | 286 |
| MeSWEET17 - cassava4.1_014640m | 287 |
| MeSWEET X - cassava4.1_031208m | 288 |
| Cucumis sativus | |
| Csativus\|Cucsa.057980\|Cucsa.057980.1 | 289 |
| Csativus\|Cucsa.057980\|Cucsa.057980.2 | 290 |
| Csativus\|Cucsa.077130\|Cucsa.077130.1 | 291 |
| Csativus\|Cucsa.077130\|Cucsa.077130.2 | 292 |
| Csativus\|Cucsa.091060\|Cucsa.091060.1 | 293 |
| Csativus\|Cucsa.098360\|Cucsa.098360.1 | 294 |
| Csativus\|Cucsa.114740\|Cucsa.114740.1 | 295 |
| Csativus\|Cucsa.114740\|Cucsa.114740.2 | 296 |
| Csativus\|Cucsa.114740\|Cucsa.114740.3 | 297 |
| Csativus\|Cucsa.134790\|Cucsa.134790.1 | 298 |
| Csativus\|Cucsa.134800\|Cucsa.134800.1 | 299 |
| Csativus\|Cucsa.157110\|Cucsa.157110.1 | 300 |
| Csativus\|Cucsa.157120\|Cucsa.157120.1 | 301 |
| Csativus\|Cucsa.181790\|Cucsa.181790.1 | 302 |
| Csativus\|Cucsa.201980\|Cucsa.201980.1 | 303 |
| Csativus\|Cucsa.252960\|Cucsa.252960.1 | 304 |
| Csativus\|Cucsa.277610\|Cucsa.277610.1 | 305 |
| Csativus\|Cucsa.277620\|Cucsa.277620.1 | 306 |
| Csativus\|Cucsa.303950\|Cucsa.303950.1 | 307 |
| Csativus\|Cucsa.339600\|Cucsa.339600.1 | 308 |
| Csativus\|Cucsa.339610\|Cucsa.339610.1 | 309 |
| Csativus\|Cucsa.349380\|Cucsa.349380.1 | 310 |
| Nicotiana attenuata | |
| Na_454_00948 | 311 |
| Na_454_01003 | 312 |
| Na_454_02704 | 313 |
| Na_454_03028 | 314 |
| Na_454_03036 | 315 |
| Na_454_03741 | 316 |
| Na_454_04103 | 317 |
| Na_454_04416 | 318 |
| Na_454_05017 | 319 |
| Na_454_05156 | 320 |
| Na_454_05391 | 321 |
| Na_454_06723 | 322 |
| Na_454_07492 | 323 |
| Na_454_16634 | 324 |
| Na_454_27848 | 325 |
| Na_454_02675 | 326 |
| Na_454_20567 | 327 |
| Phoenix dactylifera | |
| PDK_30s1148281g011 | 328 |
| PDK_30s763631g005 | 329 |
| PDK_30s763631g006 | 330 |
| PDK_30s847911g001 | 331 |
| PDK_30s668711g003 | 332 |
| PDK_30s844111g003 | 333 |
| PDK_30s1125281g002 | 334 |
| PDK_30s818661g002 | 335 |
| PDK_30s922871g007 | 336 |
| PDK_30s724061g001 | 337 |
| PDK_30s791261g004 | 338 |
| PDK_30s672781g004 | 339 |
| PDK_30s1113331g001 | 340 |
| PDK_30s1113331g002 | 341 |
| PDK_30s1113331g003 | 342 |
| PDK_30s664101g001 | 343 |
| PDK_30s759071g001 | 344 |
| PDK_30s767611g001 | 345 |
| PDK_30s669461g001 | 346 |
| PDK_30s733511g001 | 347 |
| Phaseolus vulgaris | |
| Pvulgaris\|Phvul.003G199300\|Phvul.003G199300.1 | 348 |
| Pvulgaris\|Phvul.009G162700\|Phvul.009G162700.1 | 349 |
| Pvulgaris\|Phvul.009G137700\|Phvul.009G137700.1 | 350 |
| Pvulgaris\|Phvul.009G249700\|Phvul.009G249700.1 | 351 |
| Pvulgaris\|Phvul.009G162900\|Phvul.009G162900.1 | 352 |
| Pvulgaris\|Phvul.009G134300\|Phvul.009G134300.1 | 353 |
| Pvulgaris\|Phvul.009G162800\|Phvul.009G162800.1 | 354 |
| Pvulgaris\|Phvul.005G076300\|Phvul.005G076300.2 | 355 |
| Pvulgaris\|Phvul.005G076300\|Phvul.005G076300.1 | 356 |
| Pvulgaris\|Phvul.011G168100\|Phvul.011G168100.1 | 357 |
| Pvulgaris\|Phvul.008G001100\|Phvul.008G001100.1 | 358 |
| Pvulgaris\|Phvul.008G001200\|Phvul.008G001200.1 | 359 |
| PvSWEET9-Pvulgaris\|Phvul.008G007600\|Phvul.008G007600.1 | 360 |
| Pvulgaris\|Phvul.004G017200\|Phvul.004G017200.1 | 361 |
| Pvulgaris\|Phvul.004G017400\|Phvul.004G017400.1 | 362 |
| Pvulgaris\|Phvul.004G017300\|Phvul.004G017300.1 | 363 |
| Pvulgaris\|Phvul.004G017100\|Phvul.004G017100.1 | 364 |
| Pvulgaris\|Phvul.001G061900\|Phvul.001G061900.1 | 365 |
| Pvulgaris\|Phvul.001G064300\|Phvul.001G064300.1 | 366 |
| Pvulgaris\|Phvul.006G210800\|Phvul.006G210800.1 | 367 |
| Pvulgaris\|Phvul.006G000600\|Phvul.006G000600.1 | 368 |
| Pvulgaris\|Phvul.002G283800\|Phvul.002G283800.1 | 369 |
| Pvulgaris\|Phvul.002G283900\|Phvul.002G283900.1 | 370 |
| Pvulgaris\|Phvul.002G283900\|Phvul.002G283900.2 | 371 |
| Pvulgaris\|Phvul.002G300900\|Phvul.002G300900.1 | 372 |
| Pvulgaris\|Phvul.002G203600\|Phvul.002G203600.1 | 373 |
| Ricunus communis | |
| 27985.m000892 | 374 |
| 30169.m006529 | 375 |
| 30128.m008852 | 376 |
| 29726.m004066 | 377 |
| 30068.m002528 | 378 |
| 30147.m014444 | 379 |
| 30147.m014445 | 380 |
| 29579.m000197 | 381 |
| RcSWEET9-29647.m002020 | 382 |
| 29929.m004599 | 383 |
| 29822.m003348 | 384 |
| 30147.m014447 | 385 |
| 30026.m001515 | 386 |
| 30147.m013970 | 387 |
| 29475.m000237 | 388 |
| 30147.m014446 | 389 |
| 29822.m003349 | 390 |
| 27613.m000628 | 391 |
| Prunus persica | |
| Ppersica\|ppa017677m.g\|ppa017677m | 392 |
| Ppersica\|ppa010394m.g\|ppa010394m | 393 |
| Ppersica\|ppa010181m.g\|ppa010181m | 394 |
| Ppersica\|ppa020717m.g\|ppa020717m | 395 |
| Ppersica\|ppa024244m.g\|ppa024244m | 396 |
| Ppersica\|ppa009789m.g\|ppa009789m | 397 |
| Ppersica\|ppa021855m.g\|ppa021855m | 398 |
| Ppersica\|ppa014953m.g\|ppa014953m | 399 |
| Ppersica\|ppa018792m.g\|ppa018792m | 400 |
| Ppersica\|ppa010594m.g\|ppa010594m | 401 |
| Ppersica\|ppa023718m.g\|ppa023718m | 402 |
| Ppersica\|ppa021908m.g\|ppa021908m | 403 |
| Ppersica\|ppa015264m.g\|ppa015264m | 404 |
| Ppersica\|ppa010208m.g\|ppa010208m | 405 |
| Ppersica\|ppa009422m.g\|ppa009422m | 406 |
| Ppersica\|ppa010808m.g\|ppa010808m | 407 |
| Ppersica\|ppa017165m.g\|ppa017165m | 408 |
| Ppersica\|ppa019530m.g\|ppa019530m | 409 |
| Ppersica\|ppa021919m.g\|ppa021919m | 410 |

In additional embodiments, the peptide variants described herein are functional and capable of transporting at least one sugar when used in the methods, constructs, plants and plant seeds of the present invention. In some embodiments, the SWEET variants of the present invention have an enhanced ability to transport at least one sugar compared to the wild-type SWEET.

A polypeptide having an amino acid sequence at least, for example, about 95% “identical” to a reference an amino acid sequence, e.g., SEQ ID NO: 1, is understood to mean that the amino acid sequence of the polypeptide is identical to the reference sequence except that the amino acid sequence may include up to about five modifications per each 100 amino acids of the reference amino acid sequence. In other words, to obtain a peptide having an amino acid sequence at least about 95% identical to a reference amino acid sequence, up to about 5% of the amino acid residues of the reference sequence may be deleted or substituted with another amino acid or a number of amino acids up to about 5% of the total amino acids in the reference sequence may be inserted into the reference sequence. These modifications of the reference sequence may occur at the N-terminus or C-terminus positions of the reference amino acid sequence or anywhere between those terminal positions, interspersed either individually among amino acids in the reference sequence or in one or more contiguous groups within the reference sequence.

As used herein, “identity” is a measure of the identity of nucleotide sequences or amino acid sequences compared to a reference nucleotide or amino acid sequence. In general, the sequences are aligned so that the highest order match is obtained. “Identity” per se has an art-recognized meaning and can be calculated using well known techniques. While there are several methods to measure identity between two polynucleotide or polypeptide sequences, the term “identity” is well known to skilled artisans (Carillo (1988) J. Applied Math. 48, 1073). Examples of computer program methods to determine identity and similarity between two sequences include, but are not limited to, GCG program package (Devereux (1984) Nucleic Acids Research 12, 387), BLASTP, ExPASy, BLASTN, FASTA (Atschul (1990) J. Mol. Biol. 215, 403) and FASTDB. Examples of methods to determine identity and similarity are discussed in Michaels (2011) Current Protocols in Protein Science, Vol. 1, John Wiley & Sons.

In one embodiment of the present invention, the algorithm used to determine identity between two or more polypeptides is BLASTP. In another embodiment of the present invention, the algorithm used to determine identity between two or more polypeptides is FASTDB, which is based upon the algorithm of Brutlag (1990) Comp. App. Biosci. 6, 237-245). In a FASTDB sequence alignment, the query and reference sequences are amino sequences. The result of sequence alignment is in percent identity. In one embodiment, parameters that may be used in a FASTDB alignment of amino acid sequences to calculate percent identity include, but are not limited to: Matrix=PAM, k-tuple=2, Mismatch Penalty=1, Joining Penalty=20, Randomization Group Length=0, Cutoff Score=1, Gap Penalty=5, Gap Size Penalty 0.05, Window Size=500 or the length of the subject amino sequence, whichever is shorter.

If the reference sequence is shorter or longer than the query sequence because of N-terminus or C-terminus additions or deletions, but not because of internal additions or deletions, a manual correction can be made, because the FASTDB program does not account for N-terminus and C-terminus truncations or additions of the reference sequence when calculating percent identity. For query sequences truncated at the N- or C-termini, relative to the reference sequence, the percent identity is corrected by calculating the number of residues of the query sequence that are N- and C-terminus to the reference sequence that are not matched/aligned, as a percent of the total bases of the query sequence. The results of the FASTDB sequence alignment determine matching/alignment. The alignment percentage is then subtracted from the percent identity, calculated by the above FASTDB program using the specified parameters, to arrive at a final percent identity score. This corrected score can be used for the purposes of determining how alignments “correspond” to each other, as well as percentage identity. Residues of the reference sequence that extend past the N- or C-termini of the query sequence may be considered for the purposes of manually adjusting the percent identity score. That is, residues that are not matched/aligned with the N- or C-termini of the comparison sequence may be counted when manually adjusting the percent identity score or alignment numbering.

For example, a 90 amino acid residue query sequence is aligned with a 100 residue reference sequence to determine percent identity. The deletion occurs at the N-terminus of the query sequence and therefore, the FASTDB alignment does not show a match/alignment of the first 10 residues at the N-terminus. The 10 unpaired residues represent 10% of the reference sequence (number of residues at the N- and C-termini not matched/total number of residues in the reference sequence) so 10% is subtracted from the percent identity score calculated by the FASTDB program. If the remaining 90 residues were perfectly matched (100% alignment) the final percent identity would be 90% (100% alignment—10% unmatched overhang). In another example, a 90 residue query sequence is compared with a 100 reference sequence, except that the deletions are internal deletions. In this case the percent identity calculated by FASTDB is not manually corrected, since there are no residues at the N- or C-termini of the subject sequence that are not matched/aligned with the query. In still another example, a 110 amino acid query sequence is aligned with a 100 residue reference sequence to determine percent identity. The addition in the query occurs at the N-terminus of the query sequence and therefore, the FASTDB alignment may not show a match/alignment of the first 10 residues at the N-terminus. If the remaining 100 amino acid residues of the query sequence have 95% identity to the entire length of the reference sequence, the N-terminal addition of the query would be ignored and the percent identity of the query to the reference sequence would be 95%.

As used herein, the terms “correspond(s) to” and “corresponding to,” as they relate to sequence alignment, are intended to mean enumerated positions within the reference protein, e.g., wild-type SWEET4d, and those positions in the variant or ortholog SWEET4d that align with the positions with the reference protein. Thus, when the amino acid sequence of a subject SWEET is aligned with the amino acid sequence of a reference SWEET, the amino acids in the subject sequence that “correspond to” certain enumerated positions of the reference sequence are those that align with these positions of the reference sequence, e.g., SEQ ID NO: 2, but are not necessarily in these exact numerical positions of the reference sequence. Methods for aligning sequences for determining corresponding amino acids between sequences are described herein.

Variants resulting from insertion of the polynucleotide encoding a SWEET into an expression vector system are also contemplated. For example, variants (usually insertions) may arise from when the amino terminus and/or the carboxy terminus of a SWEET is/are fused to another polypeptide.

In another aspect, the invention provides deletion variants wherein one or more amino acid residues in a SWEET are removed. Deletions can be effected at one or both termini of the SWEET, or with removal of one or more non-terminal amino acid residues of the SWEET. Deletion variants, therefore, include all functional fragments of a particular SWEET.

Within the confines of the disclosed percent identity, the invention also relates to substitution variants of disclosed polypeptides of the invention. Substitution variants include those polypeptides wherein one or more amino acid residues of a SWEET are removed and replaced with alternative residues. In one aspect, the substitutions are conservative in nature; however, the invention embraces substitutions that are also non-conservative. Conservative substitutions for this purpose may be defined as set out in the tables below. Amino acids can be classified according to physical properties and contribution to secondary and tertiary protein structure. A conservative substitution is recognized in the art as a substitution of one amino acid for another amino acid that has similar properties. Exemplary conservative substitutions are set out in below.

TABLE III
Conservative Substitutions

| Side Chain Characteristic | Amino Acid |
|---|---|
| Aliphatic | |
| Non-polar | Gly, Ala, Pro, Iso, Leu, Val |
| Polar-uncharged | Cys, Ser, Thr, Met, Asn, Gln |
| Polar-charged | Asp, Glu, Lys, Arg |
| Aromatic | His, Phe, Trp, Tyr |
| Other | Asn, Gln, Asp, Glu |

Alternatively, conservative amino acids can be grouped as described in Lehninger (1975) Biochemistry, Second Edition; Worth Publishers, pp. 71-77, as set forth below.

TABLE IV
Conservative Substitutions

| Side Chain Characteristic | Amino Acid |
|---|---|
| Non-polar (hydrophobic) | |
| Aliphatic: | Ala, Leu, Iso, Val, Pro |
| Aromatic: | Phe, Trp |
| Sulfur-containing: | Met |
| Borderline: | Gly |
| Uncharged-polar | |
| Hydroxyl: | Ser, Thr, Tyr |
| Amides: | Asn, Gln |
| Sulfhydryl: | Cys |
| Borderline: | Gly |
| Positively Charged (Basic): | Lys, Arg, His |
| Negatively Charged (Acidic) | Asp, Glu |

And still other alternative, exemplary conservative substitutions are set out below.

TABLE V
Conservative Substitutions

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

It should be understood that the definition of peptides or polypeptides of the invention is intended to include polypeptides bearing modifications other than insertion, deletion, or substitution of amino acid residues. By way of example, the modifications may be covalent in nature, and include for example, chemical bonding with polymers, lipids, other organic and inorganic moieties. Such derivatives may be prepared to improve intracellular processing, the targeting capacity of the polypeptide for desired cells or tissues and the like. Similarly, the invention further embraces SWEETs or variants thereof that have been covalently modified to include one or more water-soluble polymer attachments such as polyethylene glycol, polyoxyethylene glycol or polypropylene glycol.

The plant cell(s) utilized in methods, constructs, plants and plant seeds of the present invention can be from any part or tissue of a plant including but not limited to the root, stem, leaf, seed, seedcoat, flower, fruit, anther, nectary, ovary, petal, tapetum, xylem, or phloem. If the genetically modified plant cell is comprised within a whole plant, the entire plant need not contain or express the genetic modification.

As described herein, the genetically modified plants and/or plant cells and/or plant seeds may be a plant or from a plant that is a dicot or monocot or gymnosperm. The plant may be crops, such as a food crops, feed crops or biofuels crops. Exemplary important crops may include corn, wheat, soybean, cotton and rice. Crops also include corn, wheat, barley, triticale, soybean, cotton, millet, sorghum, sugarcane, sugar beet, potato, tomato, grapevine, citrus (orange, lemon, grapefruit, etc), lettuce, alfalfa, common bean, fava bean and strawberries, sunflowers and rapeseed, cassava, miscanthus and switchgrass. Other examples of plants include but are not limited to an African daisy, African violet, alfalfa, almond, anemone, apple, apricot, asparagus, avocado, azalea, banana and plantain, beet, bellflower, black walnut, bleeding heart, butterfly flower, cacao, caneberries, canola, carnation, carrot, cassava, diseases, chickpea, cineraria, citrus, coconut palm, coffee, common bean, maize, cotton, crucifers, cucurbit, cyclamen, dahlia, date palm, douglas-fir, elm, English walnut, flax, Acanthaceae, Agavaceae, Araceae, Araliaceae, Araucariacea, Asclepiadaceae, Bignoniaceae, Bromeliaceae, Cactaceae, Commelinaceae, Euphobiaceae, Gentianaceae, Gesneriaceae, Maranthaceae, Moraceae, Palmae, Piperaceae, Polypodiaceae, Urticaceae, Vitaceae, fuchsia, geranium, grape, hazelnut, hemp, holiday cacti, hop, hydrangea, impatiens, Jerusalem cherry, kalanchoe, lettuce, lentil, lisianthus, mango, mimulus, monkey-flower, mint, mustar, oats, papaya, pea, peach and nectarine, peanut, pear, pearl millet, pecan, pepper, Persian violet, pigeonpea, pineapple, pistachio, pocketbook plant, poinsettia, potato, primula, red clover, rhododendron, rice, rose, rye, safflower, sapphire flower, spinach, strawberry, sugarcane, sunflower, sweetgum, sweet potato, sycamore, tea, tobacco, tomato, verbena, and wild rice.

The methods, constructs, plants and plant seeds of the present invention relate to increasing levels of sugar in developing seeds. The terms “sugar” is well known in the art and is used to mean a monosaccharide, a disaccharide, a trisaccharide, a tetrasaccharide or polysaccharide. The sugar or sugars measured may or may not be modified, such as being acetylated. Specifically, the sugars that are increased are selected from the groups consisting of sucrose, fructose, glucose, mannose and galactose. The sugars that are increased may or may not be part of more complex compounds, such as trisaccharides, e.g., raffinose, tetrasaccharides, e.g., stachyose or polysaccharides, e.g., amylose, amylopectin. The invention is not limited to the identity of the specific sugars that are increased in the seeds and plants of the present invention. Indeed, the SWEET transporters of the present invention predominantly transport hexoses, such as but not limited to glucose, mannose, fructose and galactose, as well as disaccharides, such as but not limited to sucrose, lactose, maltose, trehalose, cellobiose into the developing seed. Once inside the seed coat or developing seed coat, however, the seed may utilize these increased hexoses and/or disaccharides to then form more complex sugars. These more complex sugars that may be contained (increased) in the seed or developing seed include but are not limited to disaccharides, trisaccharides, e.g., raffinose, tetrasaccharides, e.g., stachyose or polysaccharides, e.g., amylose, amylopectin.

Thus, an “increase in glucose,” for example, is used herein to mean that the levels of glucose are increased over controls, regardless of whether the glucose is free glucose, i.e., occurs as a monosaccharide, or if the glucose subunit is part of a more complex compound, such as but not limited to disaccharides, trisaccharides, tetrasaccharides, or even polysaccharides. Similarly, an “increase in fructose,” for example, is used herein to mean that the levels of fructose are increased over controls, regardless of whether the fructose is free fructose, i.e., occurs as a monosaccharide, or if the fructose subunit is part of a more complex compound, such as but not limited to disaccharides, trisaccharides, tetrasaccharides, or even polysaccharides. Similarly, an “increase in sucrose,” for example, is used herein to mean that the levels of sucrose are increased over controls, regardless of whether the sucrose is free sucrose, i.e., occurs as a disaccharide, or if the fructose is part of a more complex compound, such as but not limited to trisaccharides, tetrasaccharides, or even polysaccharides. Given that the building blocks of di-, tri-, tetra- and polysaccharides are well known, and that methods are well established for analyzing sugar content in seeds, e.g., Hirst, E. L., et al., Biochem. J., 95:453-458 (1965), Steadman, K., et al., Ann. Botany, 77:667-674 (1996), Buckeridge, M. S., Plant Physiol., 154(3):1017-1023 (2010), all of which are incorporated by reference, one of skill in the art can readily ascertain if there is an increase in the level of sugar in a seed or developing seed compared to control seeds or control developing seeds. In select embodiments, methods of assessing or measuring levels of sugar and/or starch content in seeds include but are not limited to HPLC, NMR and mass spectroscopy.

As used herein, the phase “increase in the levels at least one sugar,” or “increase at least one sugar,” or some derivation thereof, means an increase in the levels of at least one specific, measured sugar in the seed or developing seed, as compared to control seed or control developing seed, even if levels of another sugar in the seed or developing seed may decrease or remain static. Of course, more than one specific, measured sugar may be increased as compared to control seed or control developing seed. In specific embodiments, the phrase “increase in the levels of at least one sugar” means an increase in at least one of at least, glucose, mannose, fructose, galactose, sucrose, lactose, maltose, trehalose or cellobiose into the seed or developing seed. In other specific embodiments, the phrase “increase in the levels of at least one sugar” means an increase in at least two of, glucose, mannose, fructose, galactose, sucrose, lactose, maltose, trehalose or cellobiose into the seed or developing seed. In other specific embodiments, the phrase “increase in the levels of at least one sugar” means an increase in at least three of, glucose, mannose, fructose, galactose, sucrose, lactose, maltose, trehalose or cellobiose into the seed or developing seed. In other specific embodiments, the phrase “increase in the levels of at least one sugar” means an increase in at least four of, glucose, mannose, fructose, galactose, sucrose, lactose, maltose, trehalose or cellobiose into the seed or developing seed. In other specific embodiments, the phrase “increase in the levels of at least one sugar” means an increase in at least five of, glucose, mannose, fructose, galactose, sucrose, lactose, maltose, trehalose or cellobiose into the seed or developing seed. In other specific embodiments, the phrase “increase in the levels of at least one sugar” means an increase in at least six of, glucose, mannose, fructose, galactose, sucrose, lactose, maltose, trehalose or cellobiose into the seed or developing seed. In other specific embodiments, the phrase “increase in the levels of at least one sugar” means an increase in at least seven of, glucose, mannose, fructose, galactose, sucrose, lactose, maltose, trehalose or cellobiose into the seed or developing seed. In other specific embodiments, the phrase “increase in the levels of at least one sugar” means an increase in at least eight of, glucose, mannose, fructose, galactose, sucrose, lactose, maltose, trehalose or cellobiose into the seed or developing seed. In other specific embodiments, the phrase “increase in the levels of at least one sugar” means an increase in glucose, mannose, fructose, galactose, sucrose, lactose, maltose, trehalose and cellobiose into the seed or developing seed.

As used herein, the term “seed” is used as it is in the art, i.e., an embryonic plant contained in a seed coat and is generated after fertilization and at least some growth within the maternal plant. A “developing seed” is an embryonic plant that has not completed its growth within the maternal plant, or it can be an embryonic plant around which the seed coat has not completely formed. For the purposes of measuring sugars in seeds or developing seeds as that relates to the present invention described herein, the seeds or developing seeds may or may not be contained within the maternal plant. For example, the seeds may be contained within or on a fruit of the plant, and the fruit may or may not be free of the maternal plant at harvest. The location and methods of isolating the seeds or developing seeds is irrelevant for the purposes of the present invention.

The methods, constructs, plants and plant seeds of the present invention relate to inserting an exogenous nucleic acid into a plant cell, wherein the nucleic acid codes for at least one SWEET transporter protein described herein. As used herein, the phrase “exogenous nucleic acid” is used to mean a nucleic acid that normally does not exist or occur in the genome of the plant cell. For example, at least one extra copy of nucleic acid encoding a wild-type SWEET transporter is an exogenous nucleic acid. Of course copies of nucleic acids encoding mutant SWEET transporters would also be considered an exogenous nucleic acid.

In one embodiment, the exogenous nucleic acid that codes for at least one SWEET transporter protein is derived from the same species (which includes being from the same or different subspecies within the same species) in which the exogenous nucleic acid is to be inserted. For example, a nucleic acid coding for the at least one SWEET transporter protein is a nucleic acid encoding the Zea mays SWEET transporter protein and the exogenous nucleic acid is being inserted into Zea mays plant cells. In another embodiment, the exogenous nucleic acid that codes for at least one SWEET transporter protein is derived from a different species in which the exogenous nucleic acid is to be inserted. For example, a nucleic acid coding for the at least one SWEET transporter protein is a nucleic acid encoding the Arabidopsis thaliana SWEET transporter protein and the exogenous nucleic acid is being inserted into Zea mays plant cells. In yet another embodiment, the exogenous nucleic acid that codes for at least one SWEET transporter protein is derived from a different genus in which the exogenous nucleic acid is to be inserted. For example, a nucleic acid coding for the at least one SWEET transporter protein is a nucleic acid encoding the Zea perennis SWEET transporter protein and the exogenous nucleic acid is being inserted into Zea mays plant cells.

Methods for the introduction or insertion of nucleic acid molecules into plants and plant cells are well-known in the art. For example, plant transformation may be carried out using Agrobacterium-mediated gene transfer, microinjection, electroporation or biolistic methods as it is, e.g., described in Potrykus and Spangenberg (Eds.), Gene Transfer to Plants. Springer Verlag, Berlin, N.Y., 1995. Therein, and in numerous other references available to one of skill in the art, useful plant transformation vectors, selection methods for transformed cells and tissue as well as regeneration techniques are described and can be applied to the methods of the present invention.

By inserting the exogenous nucleic acid into a plant cell, a transgenic plant is thus created. The methods generally involve inserting an exogenous nucleic acid into a plant cell. The insertion may be transient such that the inserted nucleic acid is not necessarily inherited to subsequence generations. In the alternative, the insertion may be stable or integrated such that the inserted nucleic acid is inherited to subsequence generations. Moreover, the plant cell into which the nucleic acids are inserted may be in culture or it may be part of a whole plant. For example transfection of nucleic acids into plant cells, as understood herein, includes introducing nucleic acids into plant protoplasts and allowing the protoplasts to develop into a callus, which is then allowed to grow into a mature plant. As used herein, the phrase “growing the transgenic plant cell into a mature plant” is used to mean using culture or non-culture growing conditions that allow the transfected plant cell(s) to develop into a whole plant which will contain the at least one copy of the nucleic acid encoding at least one SWEET transporter protein. In other embodiments, “growing the transgenic plant cell into a mature plant” includes introducing the nucleic acid into a portion of a plant, such as a leaf, embryo or portion thereof, and subsequently regenerating a whole plant (T₀ generation) from the leaf, embryo or portion thereof. The T₀ generation plants can subsequently be mated or crossed with other plants to produce T₁, T₂, T₃, etc generations of plants. These other “mating plants” crossed with the T₀ generation plants that are used to produce subsequent generations of transgenic plants (transgenic for the SWEET transporters described herein) may or may not be wild-type plants. In another embodiment, the mating plants crossed with the T₀ generation plants that are used to produce subsequent generations of transgenic plants may or may not be transgenic plants themselves, including but not limited to another T₀ generation plant that is transgenic for at least one SWEET transporter disclosed herein). Of course, if the mating plants used to grow the transgenic plant cells into mature transgenic plants are themselves transgenic, the mating plants can be transgenic for the or different protein or nucleic acid. This subsequent crossing or mating of the T₀ generation plants into subsequent generations, e.g., T₁, T₂, T₃, etc., is included and contemplated when the phrase “growing transgenic plant cells into a mature transgenic plant” is used herein.

Once the transgenic plant cells are created, the transgenic plant cell(s) may then grow into a transgenic seed-bearing plant using methods disclosed herein and well-established in the art. The seeds produced by the transgenic seed-bearing plants then are capable of producing seeds that have increased sugar content as compared to non-transgenic plants of the same species. As used herein, a “non-transgenic plant” indicates that the plant does not have the same exogenous nucleic acid (as determined by sequence identity) encoding the SWEET protein as the transgenic plants provided herein. Thus, a non-transgenic plant, as used herein, can be a wild-type plant or it may be transgenic for a different nucleic acid, protein, mutation, etc.

As used herein, the phrase “increased levels of sugar” or “the levels are increased” is used to mean that at least one specific sugar, as defined herein, is increased when compared to control levels.

Once levels of at least one sugar are measured or assessed, either directly or indirectly, these measured levels can then be compared to control levels of the least one sugar. Control levels of sugar(s) are levels that are deemed to be levels of sugars in seeds from a non-transgenic plant (as defined herein) from the same species as the transgenic plant and grown in similar, if not the same, conditions. To establish the measured sugar levels of a non-transgenic (“normal”) plant, an individual non-transgenic plant or group of non-transgenic plants may be analyzed to determine levels of the specific sugar in the seeds that the plant or plants typically produce. The methods, constructs, compositions, plants and plant seeds of the present invention do not necessarily require that one skilled in the art actually perform the analysis to determine control levels of the at least one sugar in plants, as such data may be readily accessible in the literature or such data may be provided.

Of course, measurements of normal measured sugar levels can fall within a range of values, and values that do not fall within this “normal range” are said to be outside the normal range. These measurements may or may not be converted to a value, number, factor or score as compared to measurements in the “normal range.” For example, a specific measured value that is above the normal range may be assigned a value or +1, +2, +3, etc., depending on the scoring system devised. The comparison of the measured sugar levels to control levels is to determine if the plant seeds have elevated levels of sugar over control levels of the same sugar in the non-transgenic plants grown in the similar, if not the same, conditions.

The levels of sugar in both control and transgenic seeds can be assessed in a seed or developing seed. In one embodiment, the levels of sugar are measured in seeds or developing seeds from transgenic and non-transgenic plants when the seeds or developing seeds are at roughly the same stage of development. For example, in one embodiment, the levels of sugar are measured in seeds or developing seeds from transgenic and non-transgenic plants at the zygote stage of seed development, the pre-globular stage of seed development, the globular stage of seed development, the transition stage of seed development, the heart stage of seed development, the torpedo stage of seed development, the linear cotyledon stage of seed development, the bending cotyledon stage of seed development, or the maturation green stage of seed development. In another embodiment, the levels of sugar are measured in seeds or developing seeds from transgenic and non-transgenic plants in at least two stages selected from the zygote stage of seed development, the pre-globular stage of seed development, the globular stage of seed development, the transition stage of seed development, the heart stage of seed development, the torpedo stage of seed development, the linear cotyledon stage of seed development, the bending cotyledon stage of seed development, or the maturation green stage of seed development. In another embodiment, the levels of sugar are measured in seeds or developing seeds from transgenic and non-transgenic plants in at least three stages selected from the zygote stage of seed development, the pre-globular stage of seed development, the globular stage of seed development, the transition stage of seed development, the heart stage of seed development, the torpedo stage of seed development, the linear cotyledon stage of seed development, the bending cotyledon stage of seed development, or the maturation green stage of seed development. In another embodiment, the levels of sugar are measured in seeds or developing seeds from transgenic and non-transgenic plants in at least four stages selected from the zygote stage of seed development, the pre-globular stage of seed development, the globular stage of seed development, the transition stage of seed development, the heart stage of seed development, the torpedo stage of seed development, the linear cotyledon stage of seed development, the bending cotyledon stage of seed development, or the maturation green stage of seed development. In another embodiment, the levels of sugar are measured in seeds or developing seeds from transgenic and non-transgenic plants in at least five stages selected from the zygote stage of seed development, the pre-globular stage of seed development, the globular stage of seed development, the transition stage of seed development, the heart stage of seed development, the torpedo stage of seed development, the linear cotyledon stage of seed development, the bending cotyledon stage of seed development, or the maturation green stage of seed development. In another embodiment, the levels of sugar are measured in seeds or developing seeds from transgenic and non-transgenic plants in at least six stages selected from the zygote stage of seed development, the pre-globular stage of seed development, the globular stage of seed development, the transition stage of seed development, the heart stage of seed development, the torpedo stage of seed development, the linear cotyledon stage of seed development, the bending cotyledon stage of seed development, or the maturation green stage of seed development. In another embodiment, the levels of sugar are measured in seeds or developing seeds from transgenic and non-transgenic plants in at least seven stages selected from the zygote stage of seed development, the pre-globular stage of seed development, the globular stage of seed development, the transition stage of seed development, the heart stage of seed development, the torpedo stage of seed development, the linear cotyledon stage of seed development, the bending cotyledon stage of seed development, or the maturation green stage of seed development. In another embodiment, the levels of sugar are measured in seeds or developing seeds from transgenic and non-transgenic plants in at least eight stages selected from the zygote stage of seed development, the pre-globular stage of seed development, the globular stage of seed development, the transition stage of seed development, the heart stage of seed development, the torpedo stage of seed development, the linear cotyledon stage of seed development, the bending cotyledon stage of seed development, or the maturation green stage of seed development. In another embodiment, the levels of sugar are measured in seeds or developing seeds from transgenic and non-transgenic plants at the zygote stage of seed development, the pre-globular stage of seed development, the globular stage of seed development, the transition stage of seed development, the heart stage of seed development, the torpedo stage of seed development, the linear cotyledon stage of seed development, the bending cotyledon stage of seed development, or the maturation green stage of seed development. As understood herein, levels of sugar in seeds from transgenic plants are considered as “increased” over levels of sugar in seeds from non-transgenic plants if levels are higher in at least one of these stages of seed development.

As used herein, subject a transgenic plant cell or a transgenic plant to conditions that promote expression of the at least one SWEET transporter is understood to mean that the plant or plant cells are grown under conditions to allow expression of the exogenous nucleic acid. In many instances, such methods of subjecting a plant or plant cell to conditions to allow expression of the at least one SWEET transporter protein include normal growth (greenhouse, field, etc.) conditions. Such circumstances would include instances where the promoter used to drive expression of the nucleic acid encoding the SWEET transporter protein is not an inducible promoter, e.g., a constitutive or tissue specific promoter. In other embodiments, methods of subjecting a plant or plant cell to conditions to allow expression of the at least one SWEET transporter protein include providing a stimulus to the transgenic plant or plant cells to induce expression of the promoter that is operably linked to the nucleic acid encoding the at least one SWEET transporter protein. One of skill in the art will be able to readily recognize the conditions or stimuli that are necessary to induce a chosen inducible promoter to drive expression of a nucleic acid.

The nucleic acid encoding at least one SWEET transporter may be isolated. As used herein, the term isolated refers to molecules separated from other cell/tissue constituents (e.g. DNA or RNA) that are present in the natural source of the macromolecule. The term isolated may also refer to a nucleic acid or peptide that is substantially free of cellular material, viral material, and culture medium when produced by recombinant DNA techniques, or that is substantially free of chemical precursors or other chemicals when chemically synthesized. Moreover, an isolated nucleic acid may include nucleic acid fragments which are not naturally occurring as fragments and would not be found in the natural state.

The nucleic acids to be inserted into the plant cells may be part of an expression vector. An expression vector is one into which a desired nucleic acid sequence may be inserted by restriction and ligation such that it is operably joined or operably linked to regulatory sequences and may be expressed as an RNA transcript. Expression refers to the transcription and/or translation of an endogenous gene, transgene or coding region in a cell.

A coding sequence and regulatory sequences are operably joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of affecting transcription of that DNA sequence such that the resulting transcript might be translated into the desired protein or polypeptide.

Vectors may further contain one or more promoter sequences. A promoter may include an untranslated nucleic acid sequence usually located upstream of the coding region that contains the site for initiating transcription of the nucleic acid. The promoter region may also include other elements that act as regulators of gene expression. In further embodiments of the invention, the expression vector contains an additional region to aid in selection of cells that have the expression vector incorporated. The promoter sequence is often bounded (inclusively) at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. A promoter also optionally includes distal enhancer or repressor elements, which can be located as much as several thousand base pairs from the start site of transcription.

Activation of promoters may be specific to certain cells or tissues, for example by transcription factors only expressed in certain tissues, or the promoter may be ubiquitous and capable of expression in most cells or tissues.

A constitutive promoter is a promoter that is active under most environmental and developmental conditions. An inducible promoter is a promoter that is active under certain or specific environmental or developmental regulation. Any inducible promoter can be used, see, e.g., Ward et al. Plant Mol. Biol. 22:361-366, 1993. Exemplary inducible promoters include, but are not limited to, that from the ACEI system (responsive to copper) (Meft et al. Proc. Natl. Acad. Sci. USA 90:4567-4571, 1993, In2 gene from maize (responsive to benzenesulfonamide herbicide safeners) (Hershey et al. Mol. Gen. Genetics 227:229-237, 1991, and Gatz et al. Mol. Gen. Genetics 243:32-38, 1994) or Tet repressor from Tn10 (Gatz et al. Mol. Gen. Genetics 227:229-237, 1991). The inducible promoter may respond to an agent foreign to the host cell, see, e.g., Schena et al. PNAS 88: 10421-10425, 1991. Other promoters include but are not limited to waxy 1 (“wx1”) promoter active in starchy endosperm tissue, the BETL1 promoter, Esr6a and 6b promoters and the Miniature1 (Mn1) promoter.

The inserted exogenous nucleic acid encoding at least one SWEET transporter may be expressed in any location in the cell, including the cytoplasm, cell surface or subcellular organelles such as the nucleus, vesicles, ER, vacuole, etc. Methods and vector components for targeting the expression of proteins to different cellular compartments are well known in the art, with the choice dependent on the particular cell or organism in which the transporter is expressed. See, for instance, Okumoto et al. PNAS 102: 8740-8745, 2005, Fehr et al. J. Fluoresc. 14: 603-609, 2005. Transport of protein to a subcellular compartment such as the chloroplast, vacuole, peroxisome, glyoxysome, cell wall or mitochondrion or for secretion into the apoplast, may be accomplished by means of operably linking a nucleotide sequence encoding a signal sequence to the 5′ and/or 3′ region of a gene encoding the transporter. Targeting sequences at the 5′ and/or 3′ end of the structural gene may determine during protein synthesis and processing where the encoded protein is ultimately compartmentalized.

The presence of a signal sequence directs a polypeptide to either an intracellular organelle or subcellular compartment or for secretion to the apoplasm. The term targeting signal sequence refers to amino acid sequences, the presence of which in or appended to an expressed protein targets it to a specific subcellular localization. For example, corresponding targeting signals may lead to the secretion of the expressed SWEET transporter, e.g. from a bacterial host in order to simplify its purification. In one embodiment, targeting of the transporter may be used to affect the concentration of at least one sugar in a specific subcellular or extracellular compartment. Appropriate targeting signal sequences useful for different groups of organisms are known to the person skilled in the art and may be retrieved from the literature or sequence data bases.

If targeting to the plastids of plant cells is desired, a targeting signal peptide can be used. An example of a targeting signal peptide includes but is not limited to amino acid residues 1 to 124 of Arabidopsis thaliana plastidial RNA polymerase (AtRpoT 3) (Plant Journal 17: 557-561, 1999), the targeting signal peptide of the plastidic Ferredoxin:NADP+ oxidoreductase (FNR) of spinach (Jansen et al. Current Genetics 13: 517-522, 1988), the amino acid sequence encoded by the nucleotides −171 to 165 of the cDNA sequence disclosed therein, the transit peptide of the waxy protein of maize including or without the first 34 amino acid residues of the mature waxy protein (Klosgen et al. Mol. Gen. Genet. 217: 155-161, 1989), the signal peptides of the ribulose bisphosphate carboxylase small subunit (Wolter et al. PNAS 85: 846-850, 1988; Nawrath et al. PNAS 91: 12760-12764, 1994), the signal peptide of the NADP malat dehydrogenase (Gallardo et al. Planta 197: 324-332, 1995), the signal peptide of the glutathione reductase (Creissen et al. Plant J. 8: 167-175, 1995) or the signal peptide of the R1 protein (Lorberth et al. Nature Biotechnology 16: 473-477, 1998).

Targeting to the mitochondria of plant cells may be accomplished by using targeting signal peptides such as but not limited to amino acid residues 1 to 131 of Arabidopsis thaliana mitochondrial RNA polymerase (AtRpoT 1) (Plant Journal 17: 557-561, 1999) or the transit peptide described by Braun (EMBO J. 11: 3219-3227, 1992).

Targeting to the vacuole in plant cells may be achieved by using targeting signal peptides such as but not limited to the N-terminal sequence (146 amino acids) of the patatin protein (Sonnewald et al. Plant J. 1: 95-106, 1991), the signal sequences described by Matsuoka and Neuhaus (Journal of Exp. Botany 50: 165-174, 1999), Chrispeels and Raikhel (Cell 68: 613-616, 1992), Matsuoka and Nakamura (PNAS 88: 834-838, 1991), Bednarek and Raikhel (Plant Cell 3: 1195-1206, 1991) and/or Nakamura and Matsuoka (Plant Phys. 101: 1-5, 1993).

Targeting to the ER in plant cells may be achieved by using, e.g., the ER targeting peptide HKTMLPLPLIPSLLLSLSSAEF in conjunction with the C-terminal extension HDEL (Haselhoff, PNAS 94: 2122-2127, 1997). Targeting to the nucleus of plant cells may be achieved by using, e.g., the nuclear localization signal (NLS) of the tobacco C2 polypeptide QPSLKRMKIQPSSQP (SEQ ID NO: 411).

Targeting to the extracellular space may be achieved by using a transit peptide such as but not limited to the signal sequence of the proteinase inhibitor II-gene (Keil et al. Nucleic Acid Res. 14: 5641-5650, 1986, von Schaewen et al. EMBO J. 9: 30-33, 1990), of the levansucrase gene from Erwinia amylovora (Geier and Geider, Phys. Mol. Plant Pathol. 42: 387-404, 1993), of a fragment of the patatin gene B33 from Solanum tuberosum, which encodes the first 33 amino acids (Rosahl et al. Mol Gen. Genet. 203: 214-220, 1986) or of the one described by Oshima et al. (Nucleic Acids Res. 18: 181, 1990).

Additional targeting to the plasma membrane of plant cells may be achieved by fusion to a different transporter, preferentially to the sucrose transporter SUT1 (Riesmeier, EMBO J. 11: 4705-4713, 1992). Targeting to different intracellular membranes may be achieved by fusion to membrane proteins present in the specific compartments such as vacuolar water channels (γTIP) (Karlsson, Plant J. 21: 83-90, 2000), MCF proteins in mitochondria (Kuan, Crit. Rev. Biochem. Mol. Biol. 28: 209-233, 1993), triosephosphate translocator in inner envelopes of plastids (Flugge, EMBO J. 8: 39-46, 1989) and photosystems in thylacoids.

Targeting to the Golgi apparatus can be accomplished using the C-terminal recognition sequence K(X)KXX where “X” is any amino acid (Garabet, Methods Enzymol. 332: 77-87, 2001. Targeting to the peroxisomes can be done using the peroxisomal targeting sequence PTS I or PTS II (Garabet, Methods Enzymol. 332: 77-87, 2001).

SWEETs Involvement in Seed Filling

Although no SWEET involvement has been found in embryos, overexpression of other sugar transporters, such as the tonoplast monosaccharide transporter (TMT1), under the control of a constitutive cauliflower mosaic virus 35S promoter, has been shown to increase biomass of Arabidopsis seeds. See Wingenter, K., et al., Plant Physiol., 154(2): 665-677 (October 2010), which is incorporated by reference. In particular, Wingenter et al. showed that increasing expression of the TMT1 transporter increased lipid and protein content in Arabidopsis seeds. Specifically, Arabidopsis overexpressing TMT1 grew faster than wild-type plants on soil and in high-glucose (Glc)-containing liquid medium. Soil-grown TMT1 overexpressor mutants produced larger seeds and greater total seed yield, which was associated with increased lipid and protein content. These changes in seed properties were correlated with slightly decreased nocturnal CO₂ release and increased sugar export rates from detached source leaves. Thus, increased TMT activity in Arabidopsis induced modified subcellular sugar compartimentation, altered cellular sugar sensing, affected assimilate allocation, increased the biomass of Arabidopsis seeds, and accelerated early plant development.

In other contexts, Rossi, G., et al., Microbial Cell Factories, 9:15 (March 2010) (doi:10.1186/1475-2859-9-15) also reports that yeast cells engineered to overexpress the hexose transporter HXT1 or HXT7, lead to increased in glucose uptake in the cells. In still other contexts, Wang et al. (2008) reports that the rice GIF1 (Grain Incomplete Filling 1) gene encoding a cell-wall invertase is required for carbon partitioning during early grain-filling. Ectopic expression of the cultivated GIF1 gene with the 35S or rice Waxy promoter resulted in smaller grains, whereas overexpression of GIF1, driven by its native promoter, increased grain production. These findings, together with the domestication signature, which were identified by comparing nucleotide diversity of the GIF1 loci between cultivated and wild rice, strongly suggest that GIF1 is a potential domestication gene and that such a domestication-selected gene can be used for further crop improvement.

Analysis of cell-specific expression in developing seeds is consistent with a role of several SWEETs in sugar import into developing seeds. Analysis of public databases and prior publications indicates that Arabidopsis SWEET1, 4, 5, 7 and 8 (Clade I and II hexose transporters), are expressed in seeds during seed maturation: SWEET1 and 7 in seed coat, SWEET8 in endosperm, and SWEETS in embryo. See Chen, L. Q., et al., Nature, 468:527-532 (2010), which is incorporated by reference. Moreover, SWEET10, 11, 12 and 15 (Clade III sucrose transporters) are expressed in seeds during maturation, specifically SWEET11, 12 and 15 in seed coat, SWEET10 in the chalazal seed coat, and SWEET11 and 15 in the endosperm. See Chen, L. Q., et al., Science, 335:207-211 (January 2012).

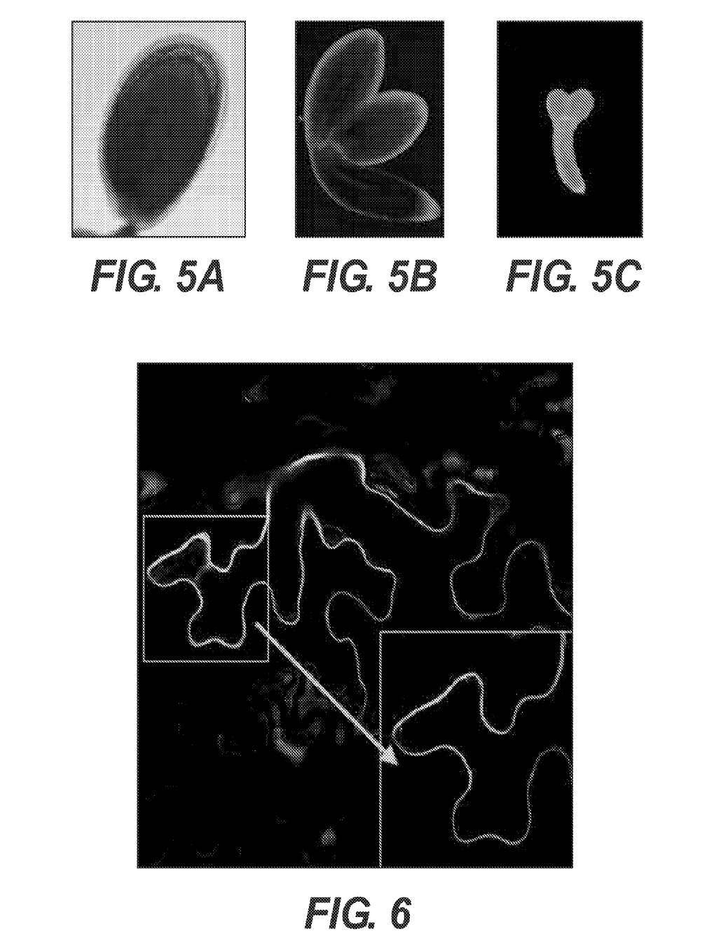

Analysis provided herein confirms that GFP-fusions of SWEET11, 12 and 15 are expressed in seed coat (FIG. 5A). Moreover, a triple sweet11, sweet12, sweet15 mutant shows retarded development and reduced starch content (FIG. 5B, C). Members of the SUT/SUCs proton sucrose cotransporter family are also expressed in the seed coat. Specifically, SUC2, 3, 4, and 5 are expressed during seed maturation, with SUC2 specifically during the ‘maturation green stage’, and SUCS during the linear cotyledon stage.

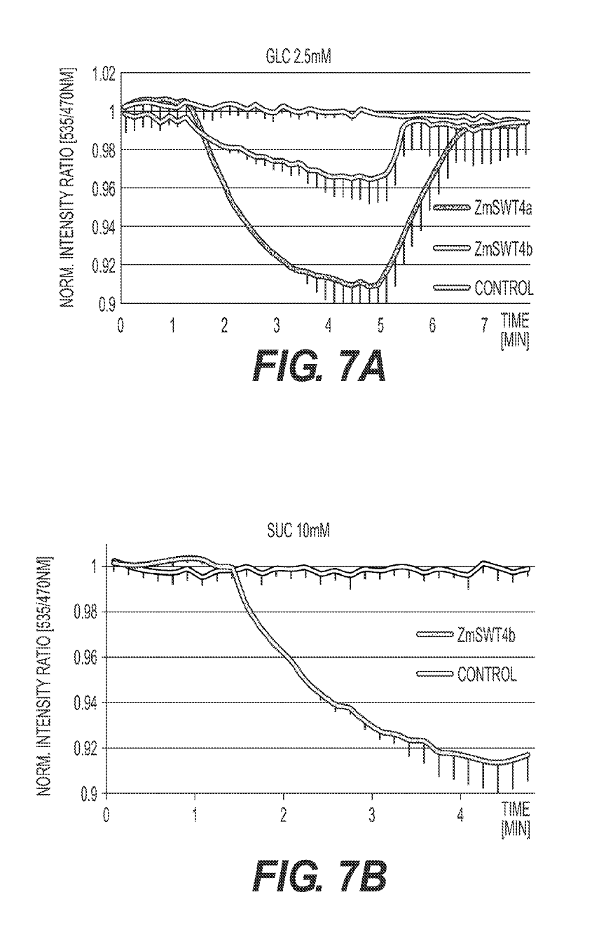

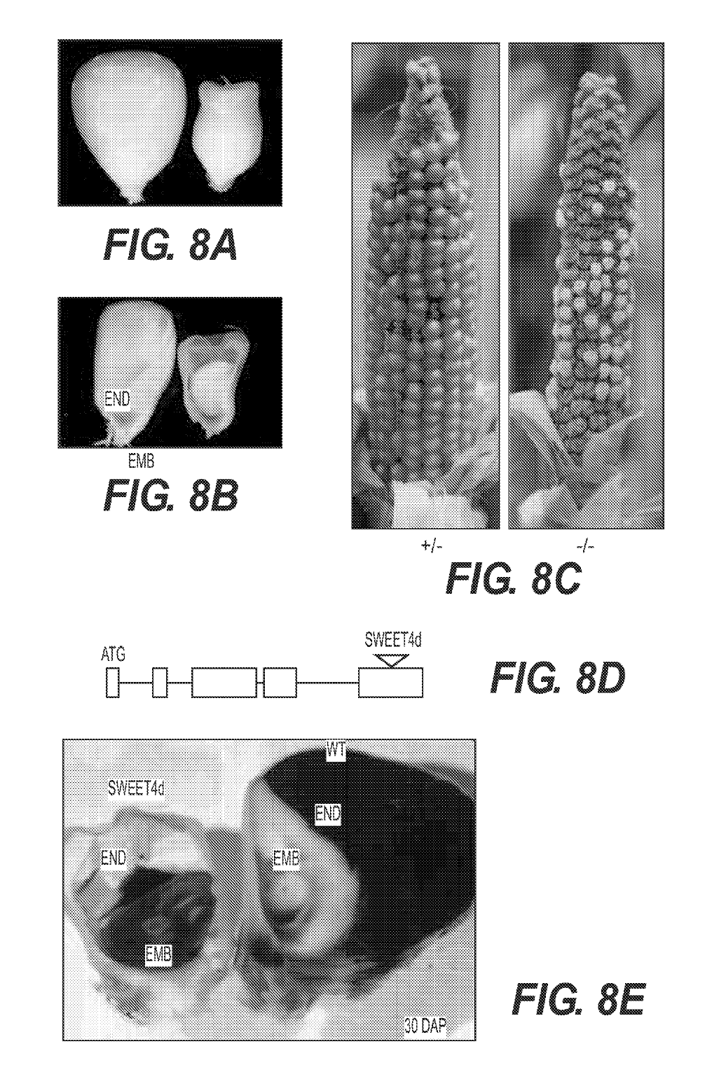

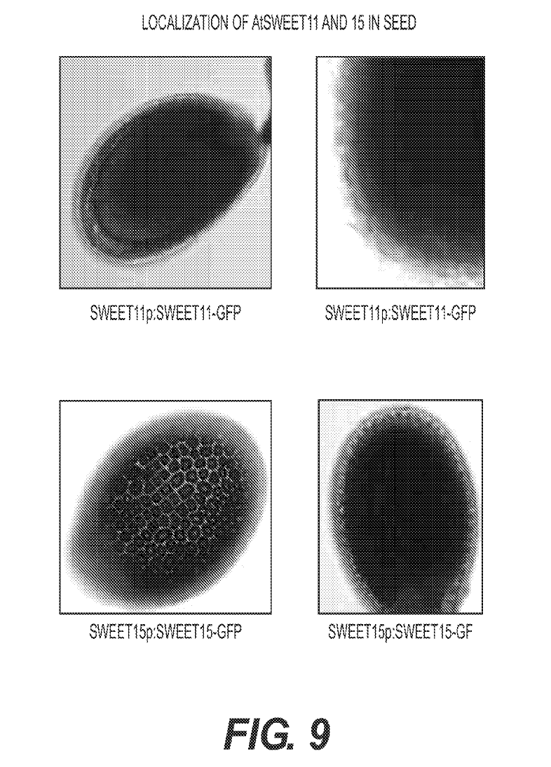

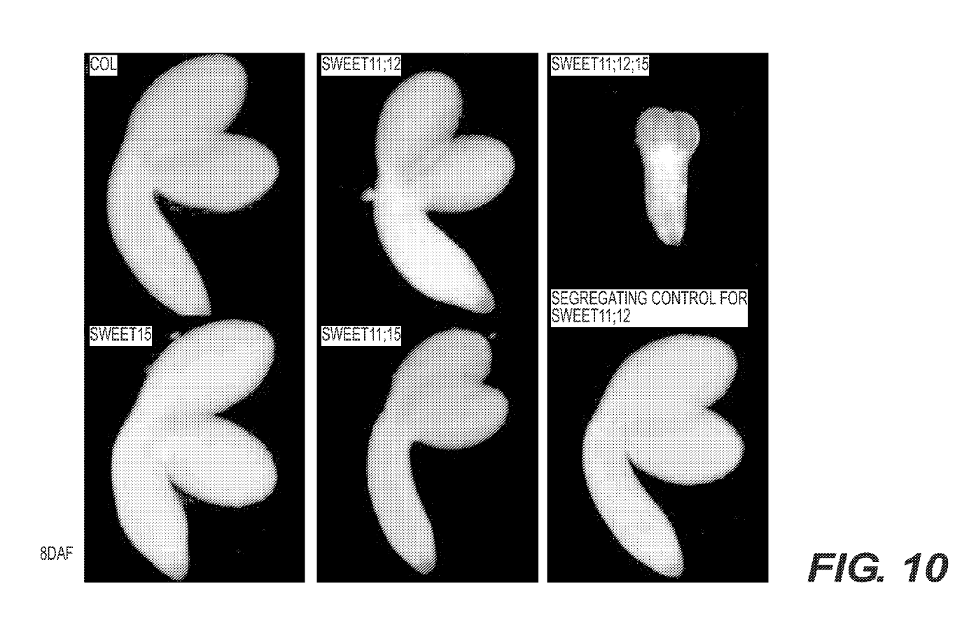

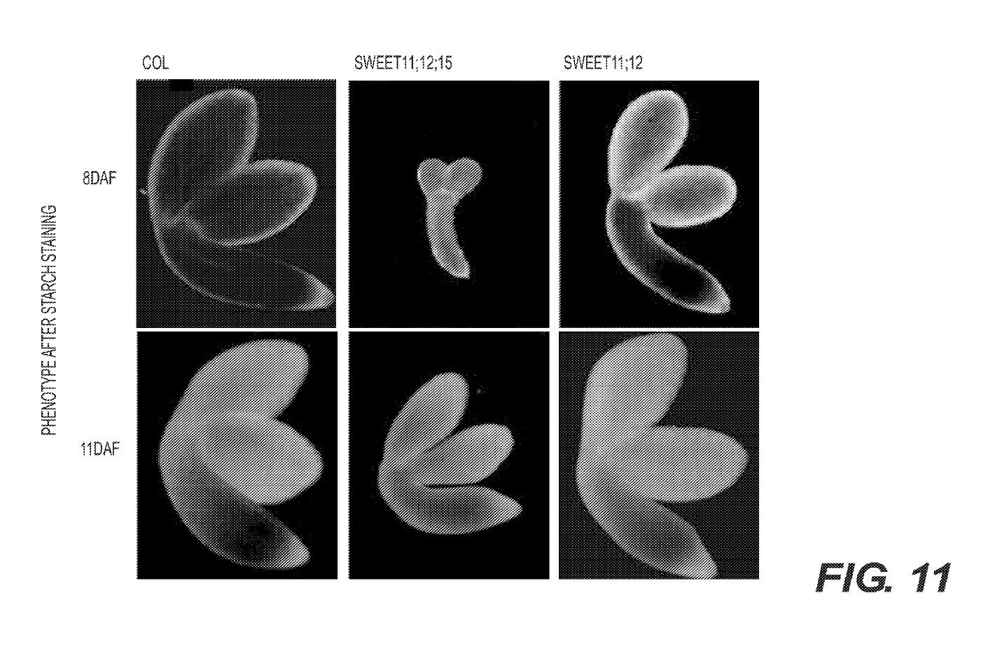

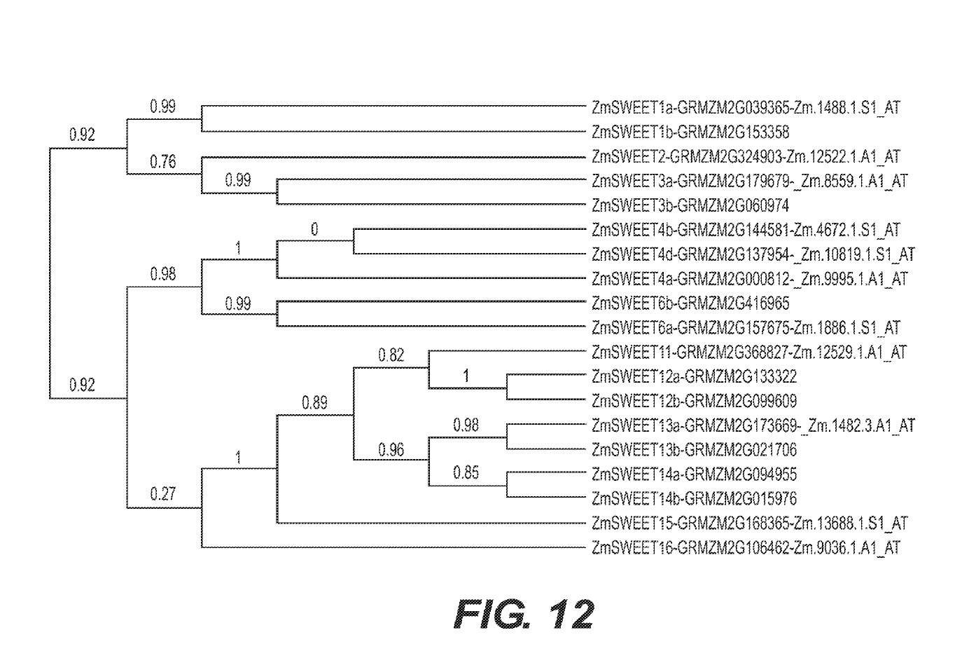

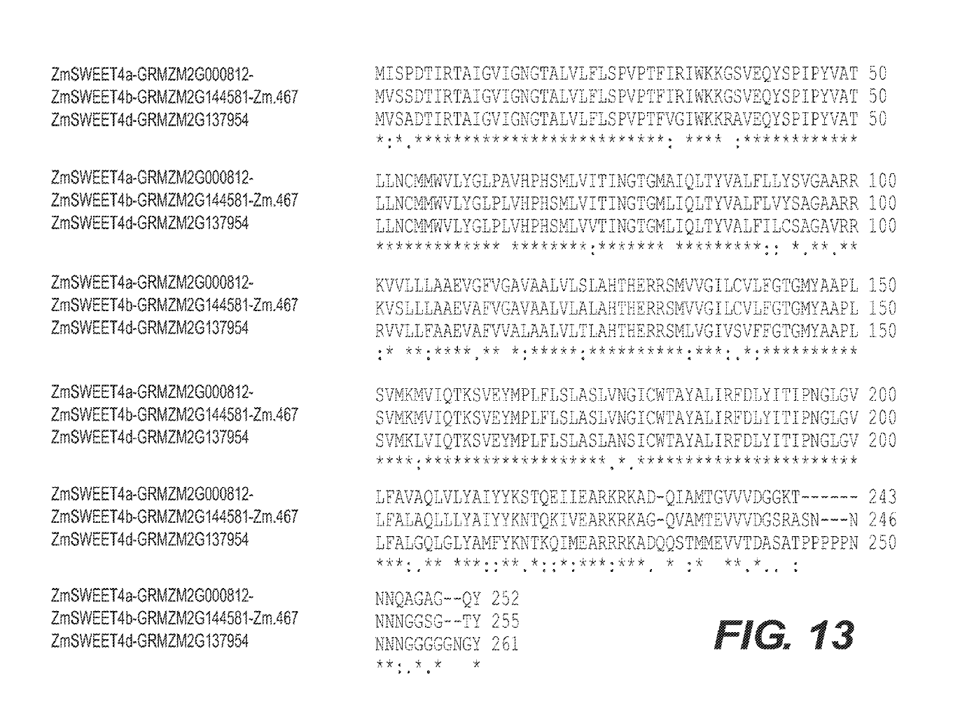

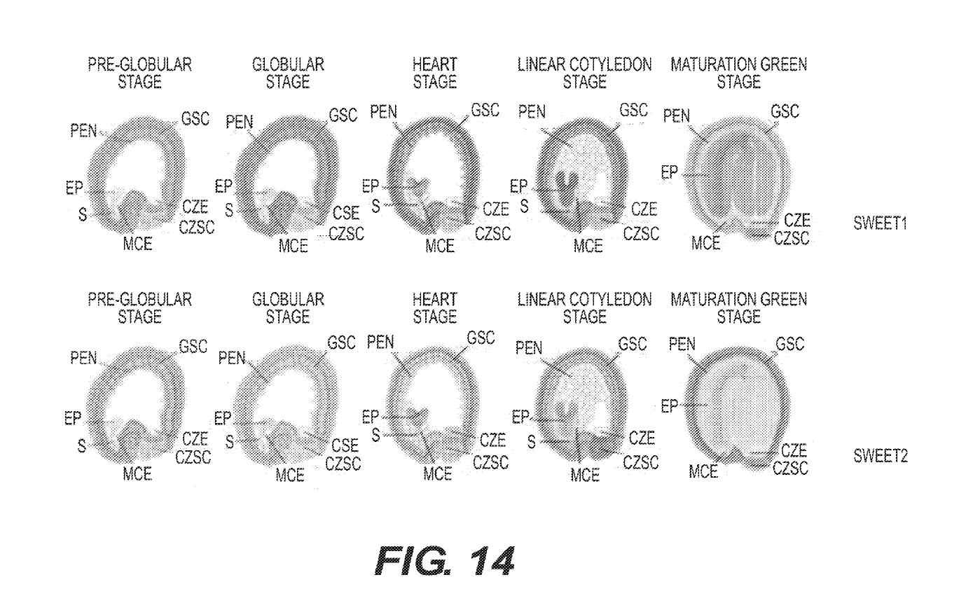

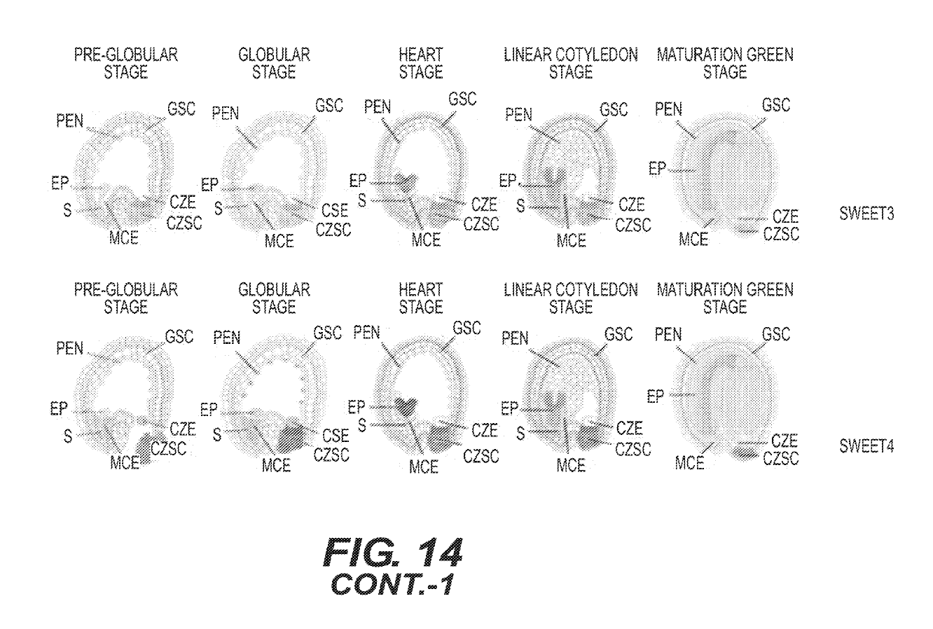

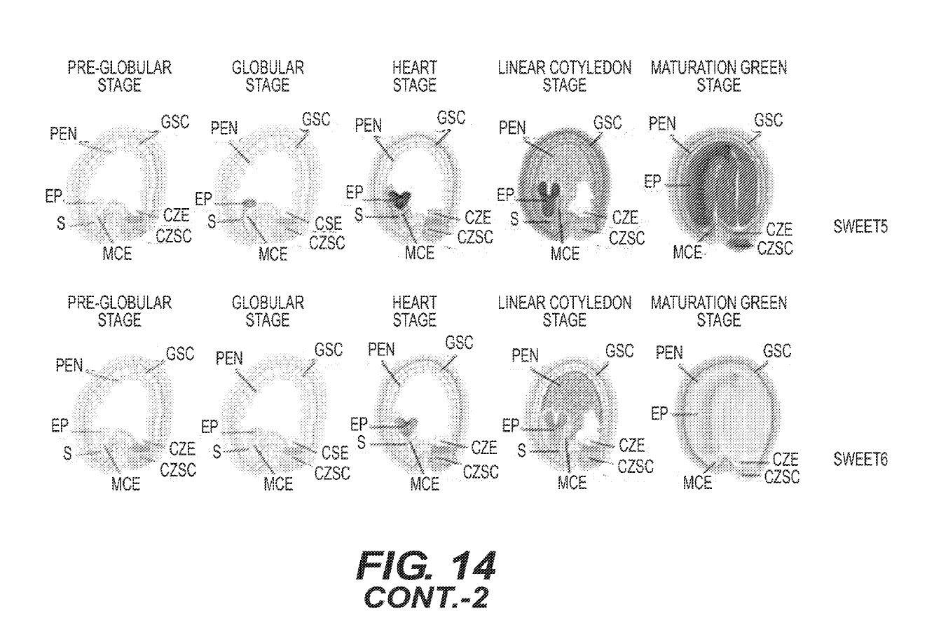

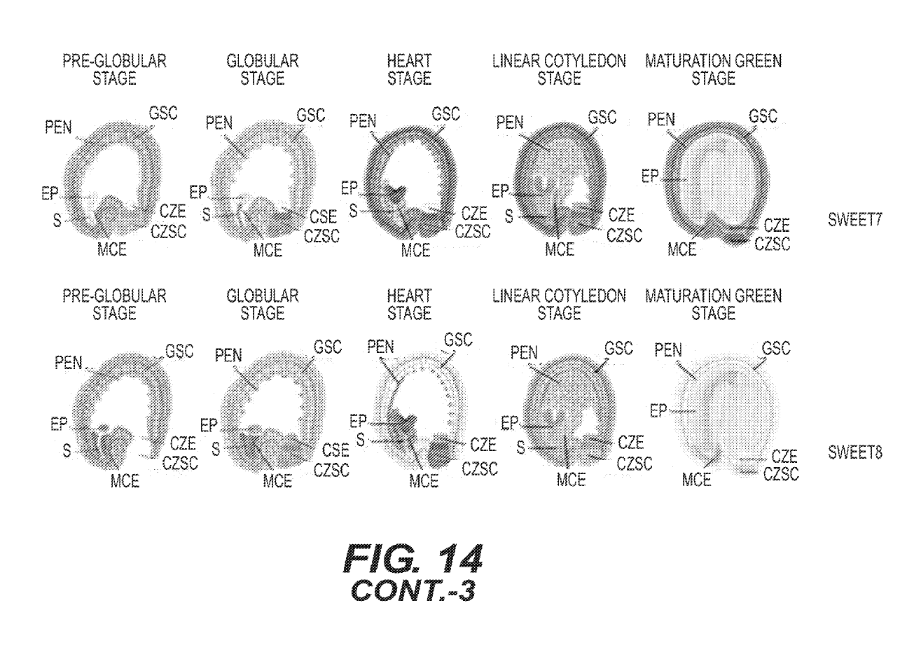

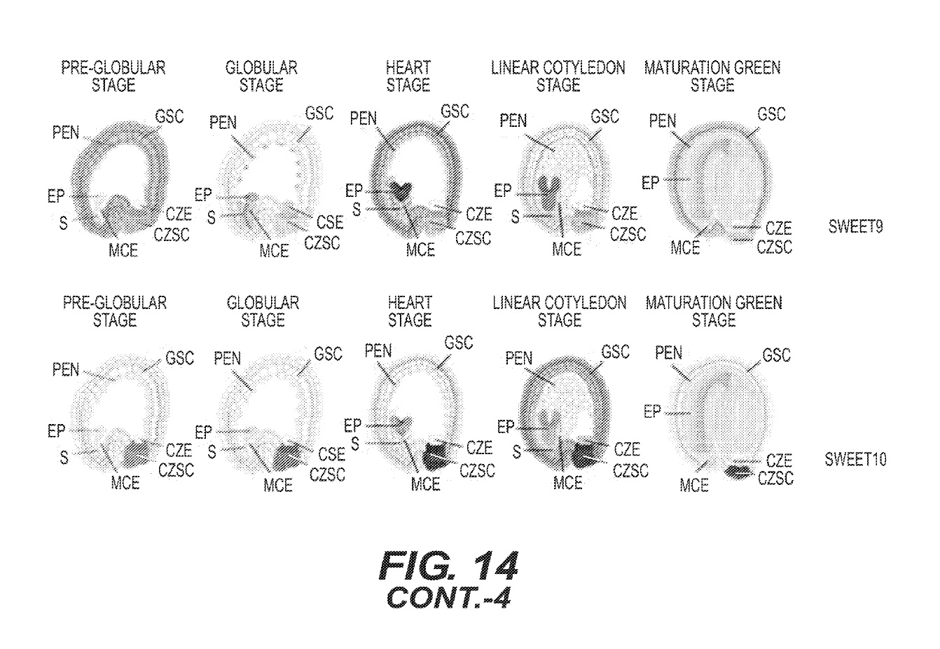

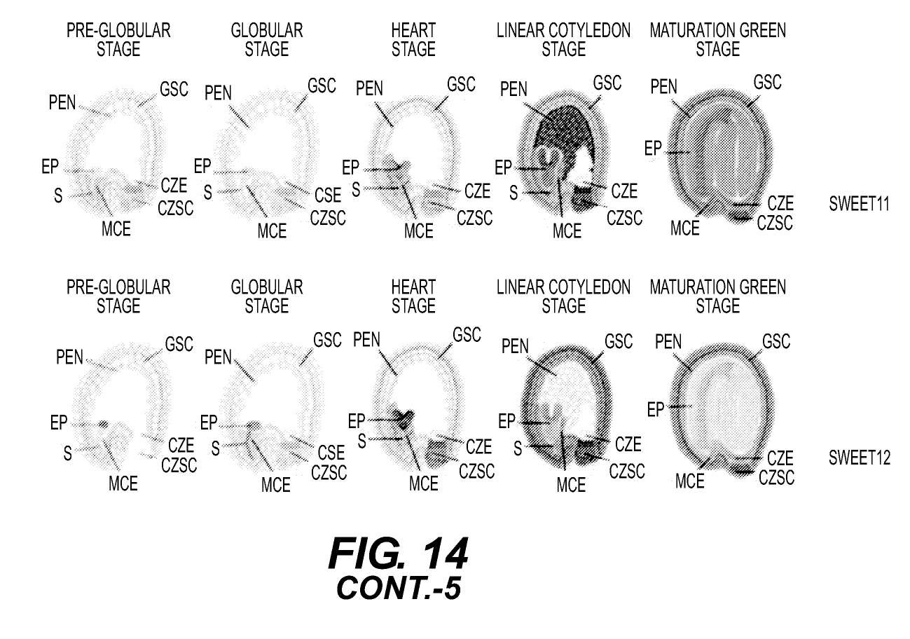

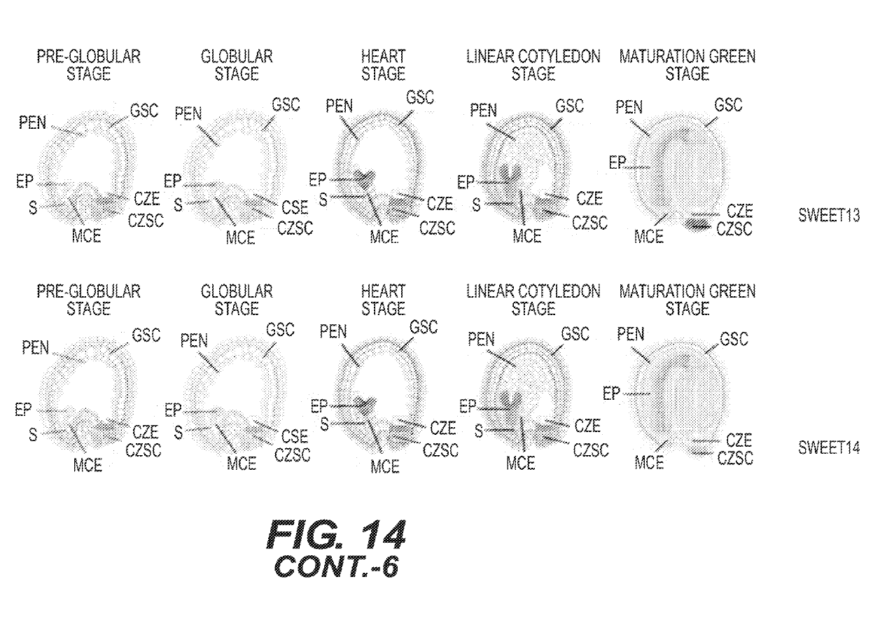

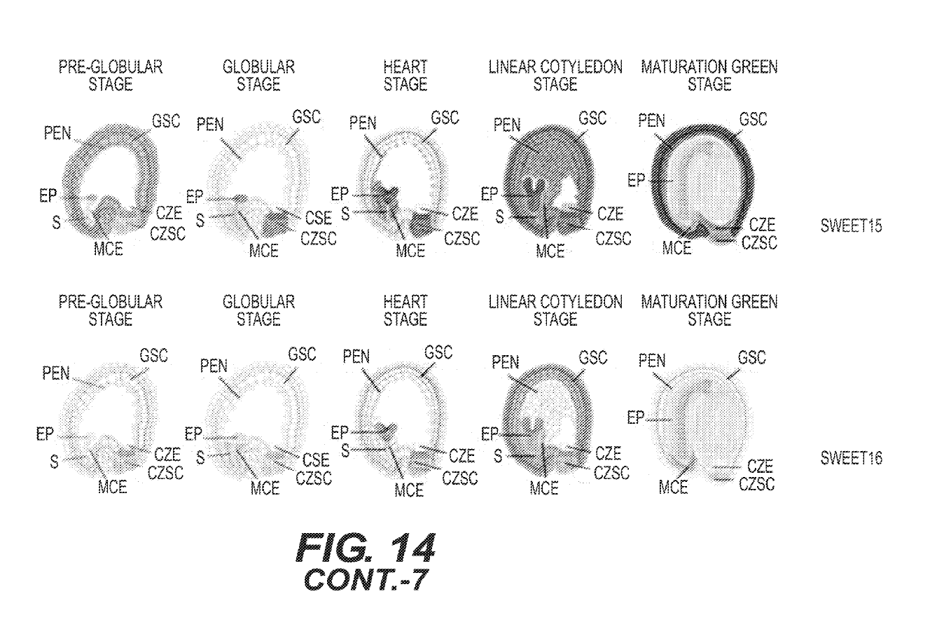

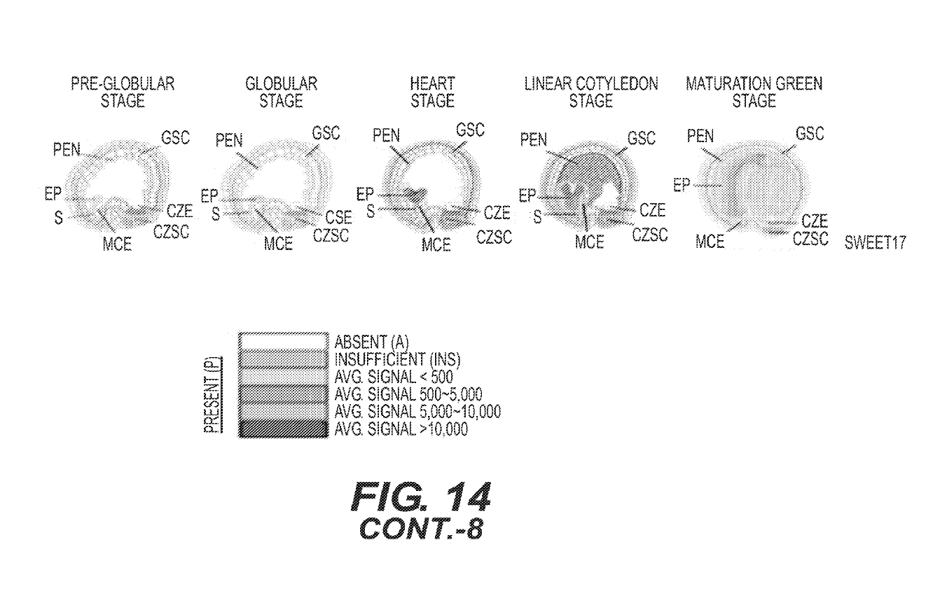

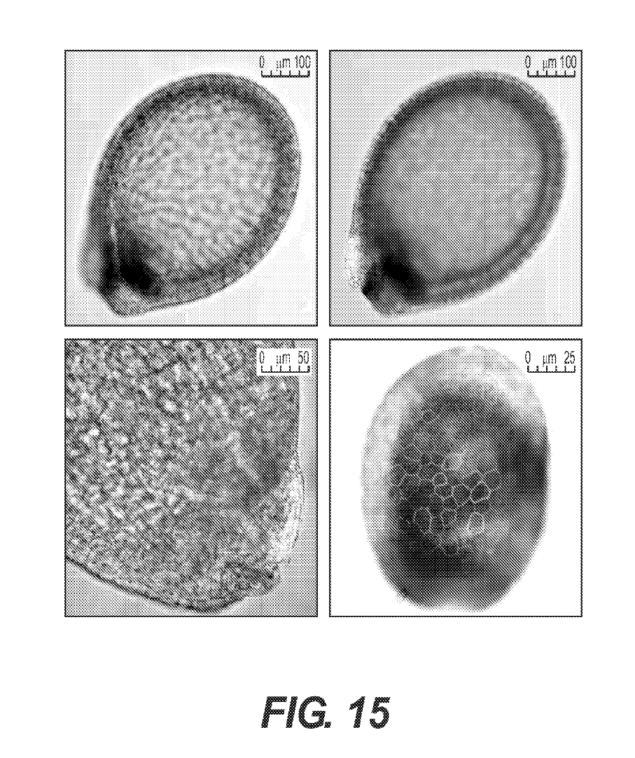

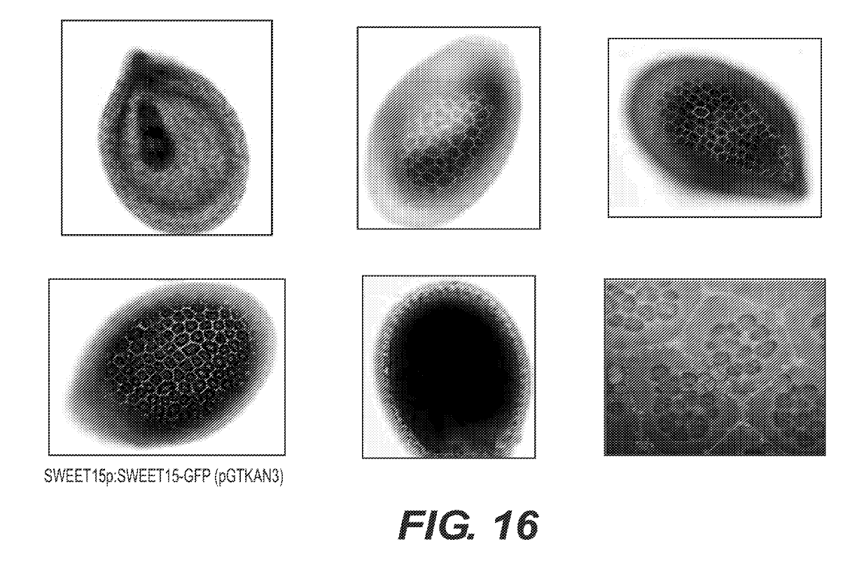

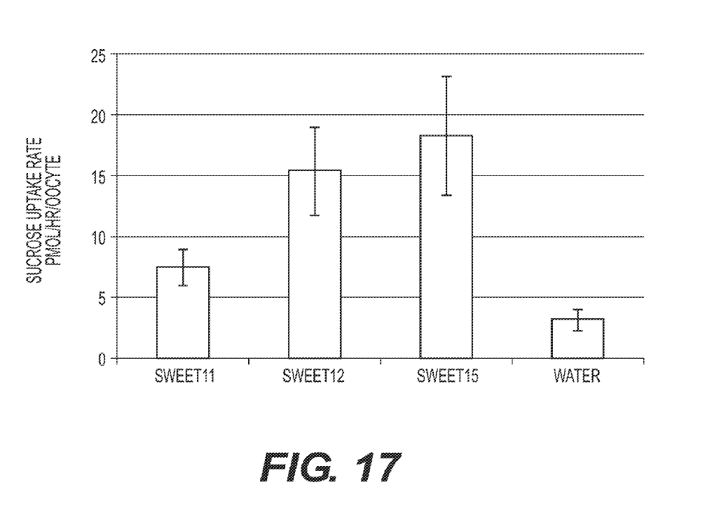

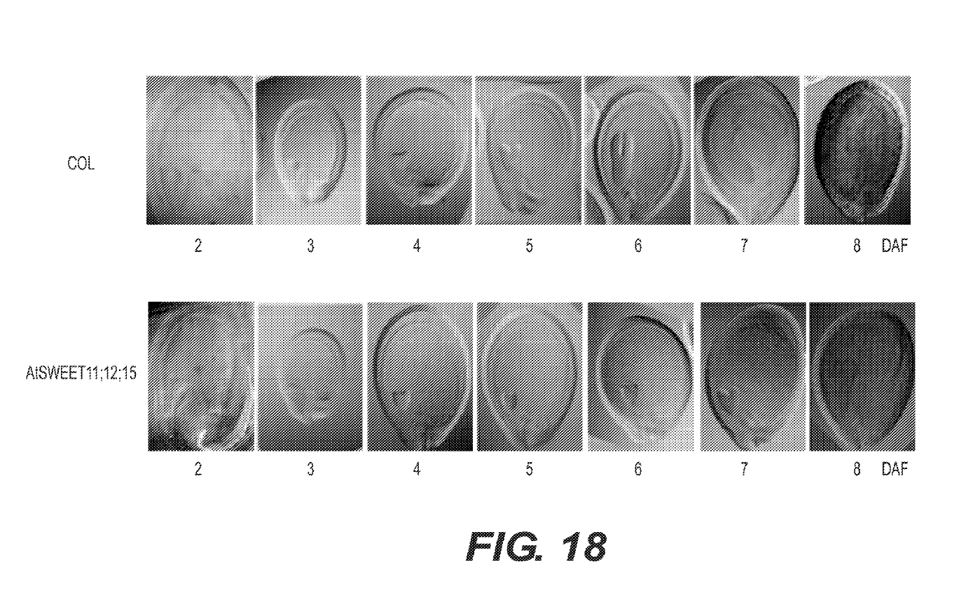

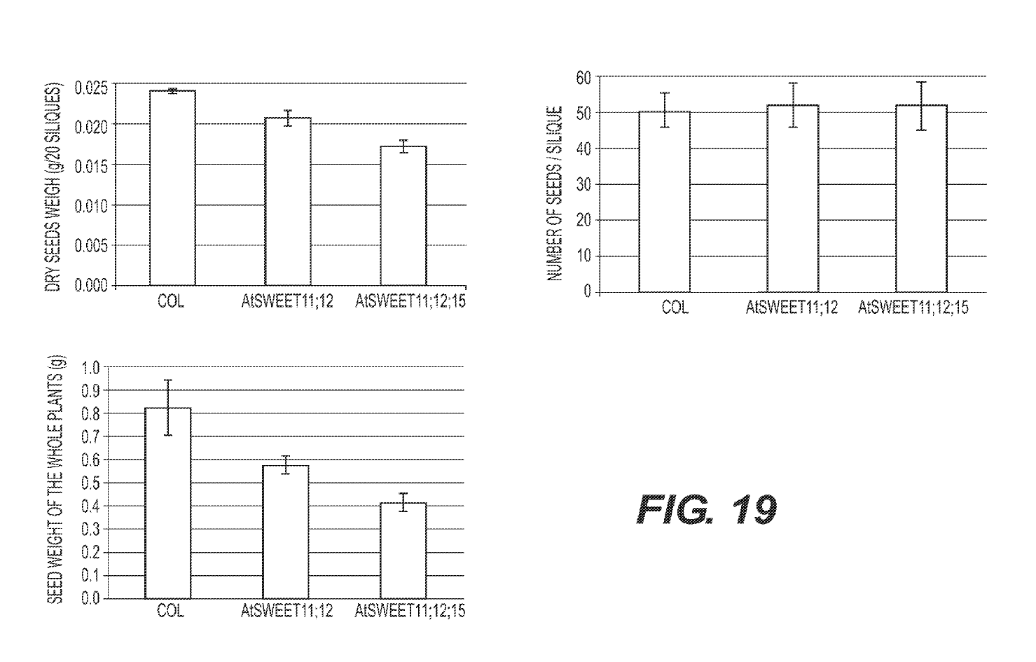

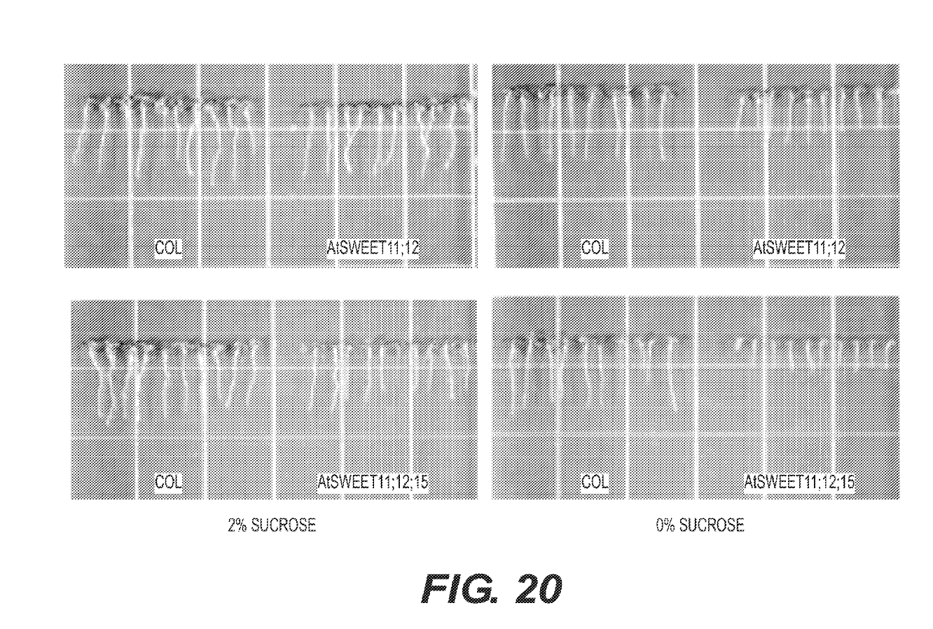

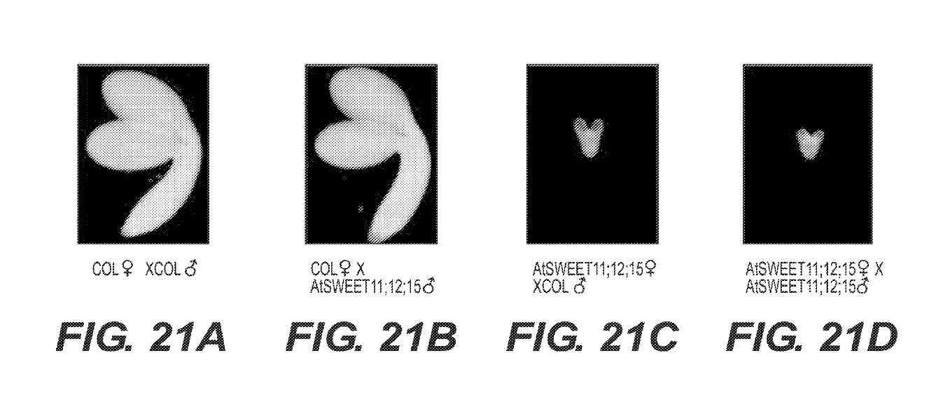

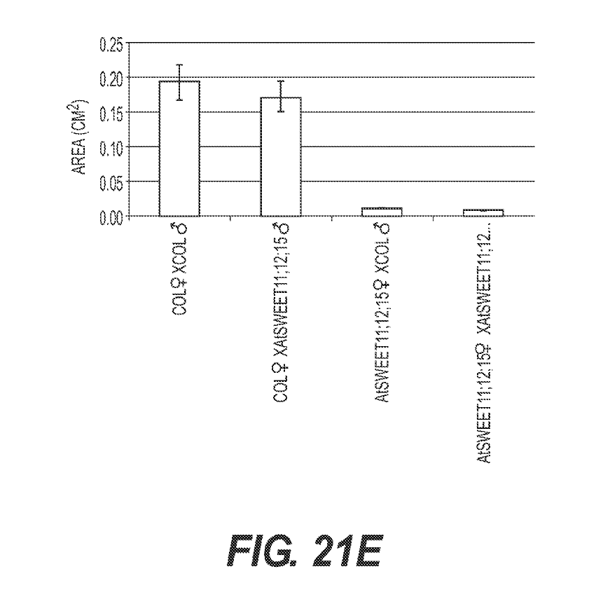

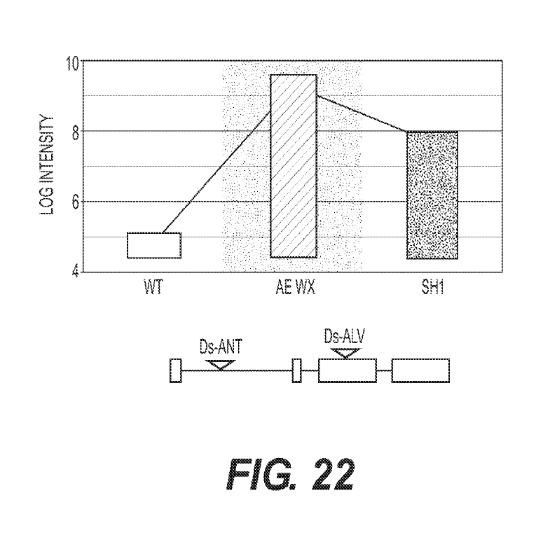

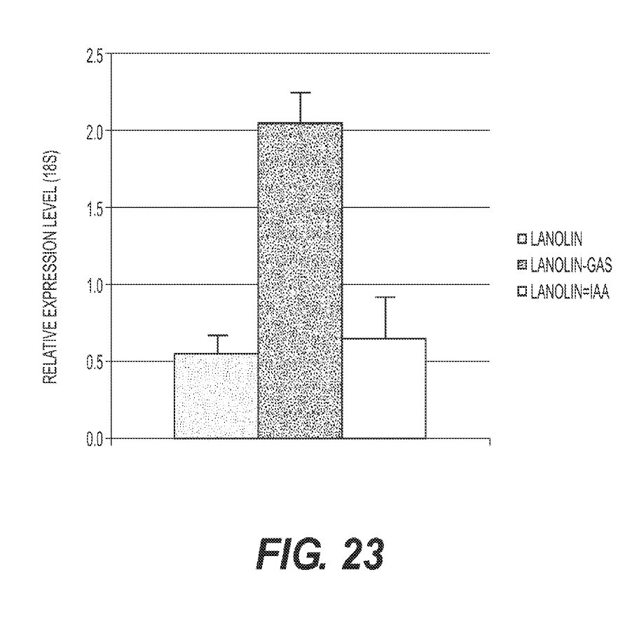

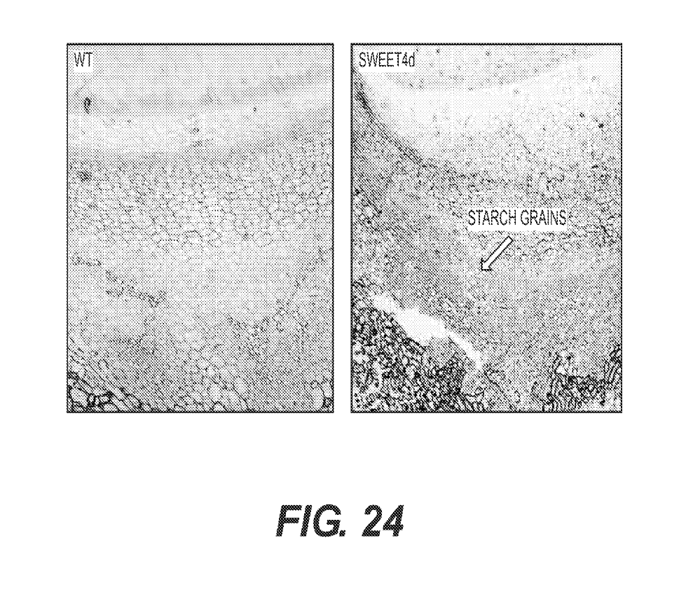

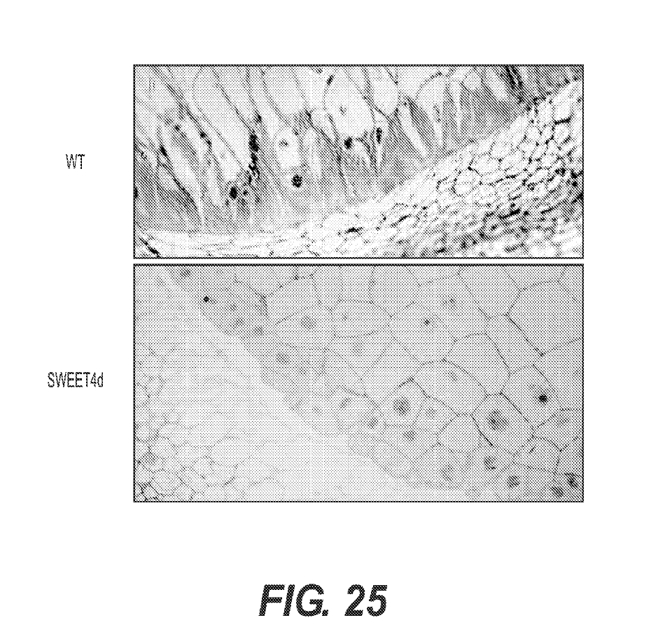

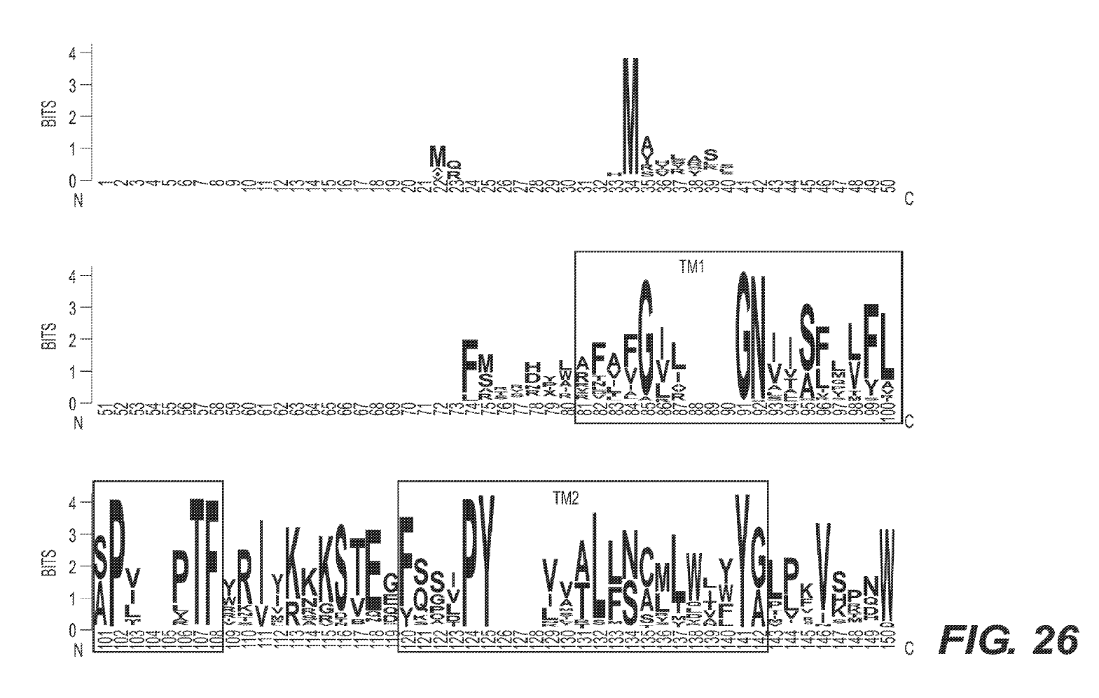

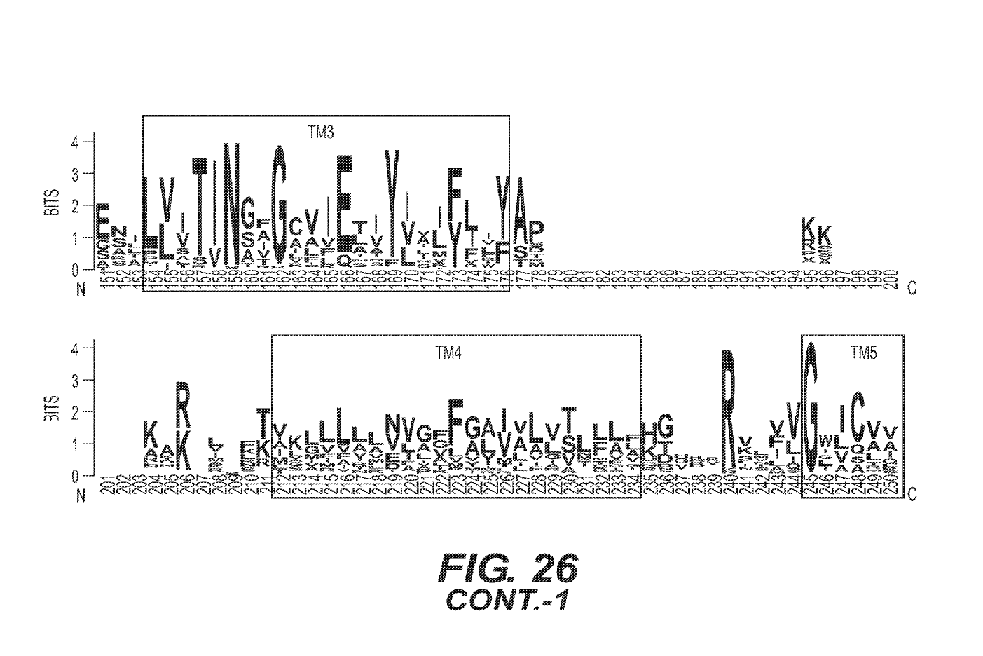

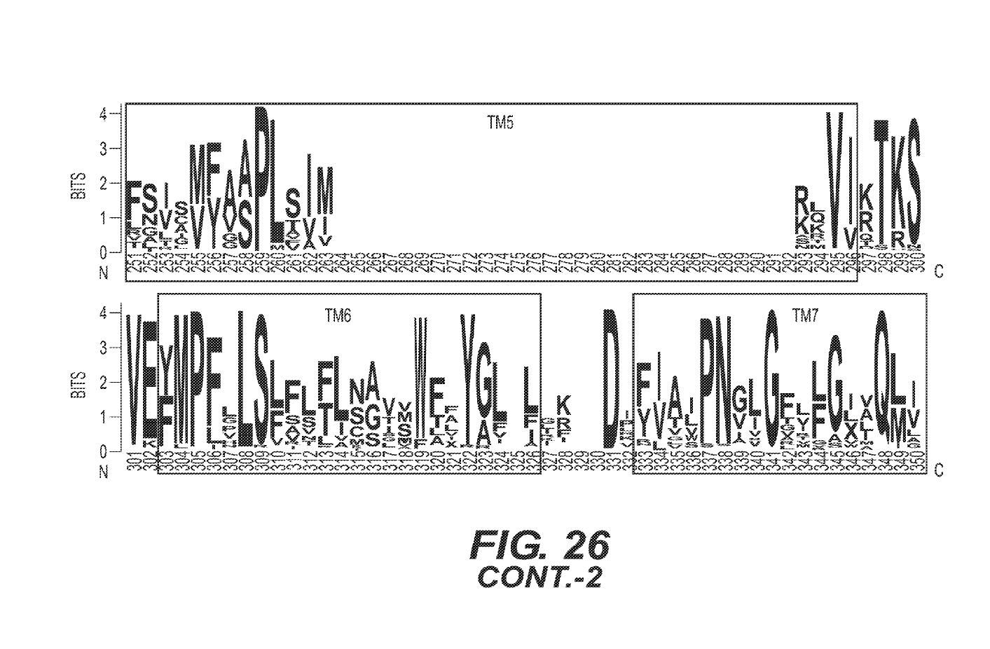

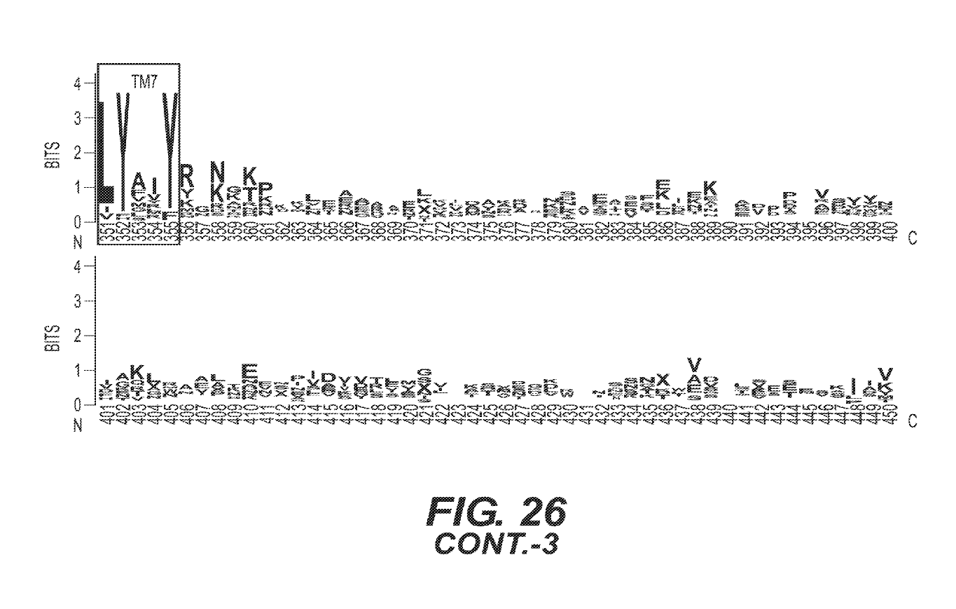

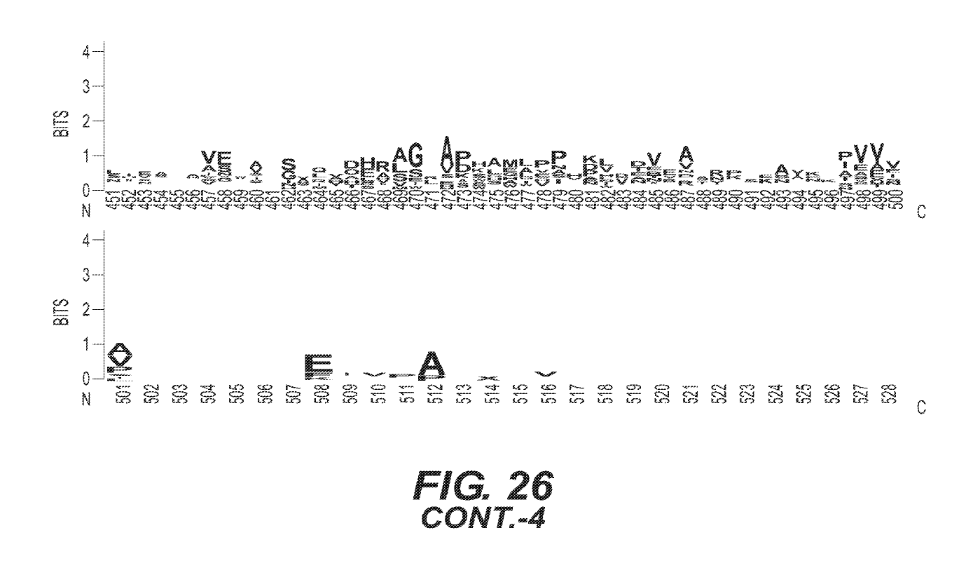

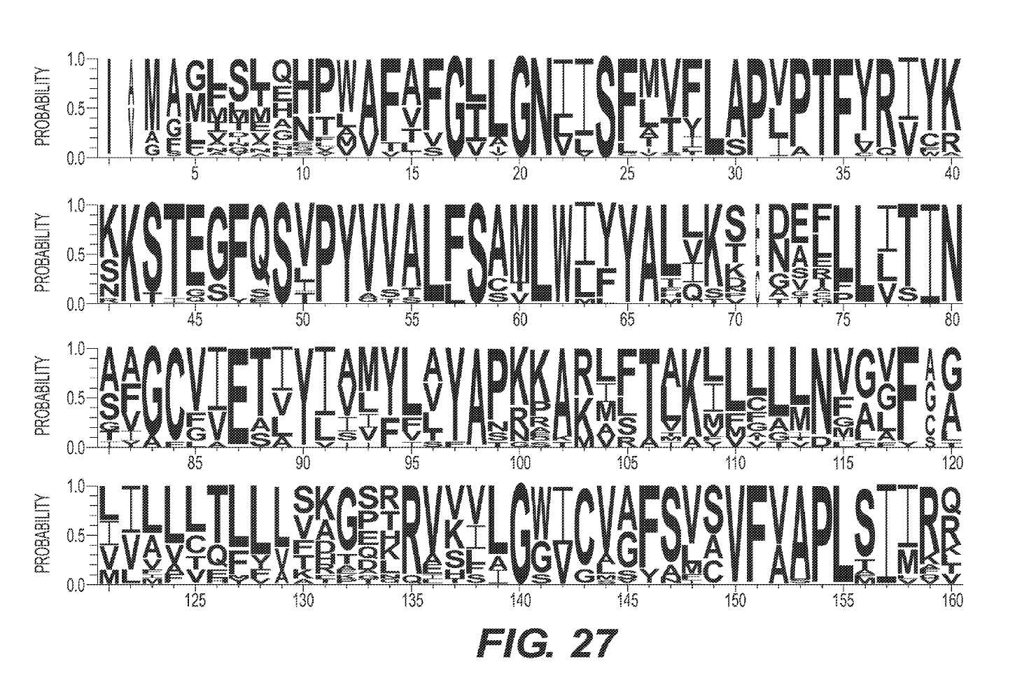

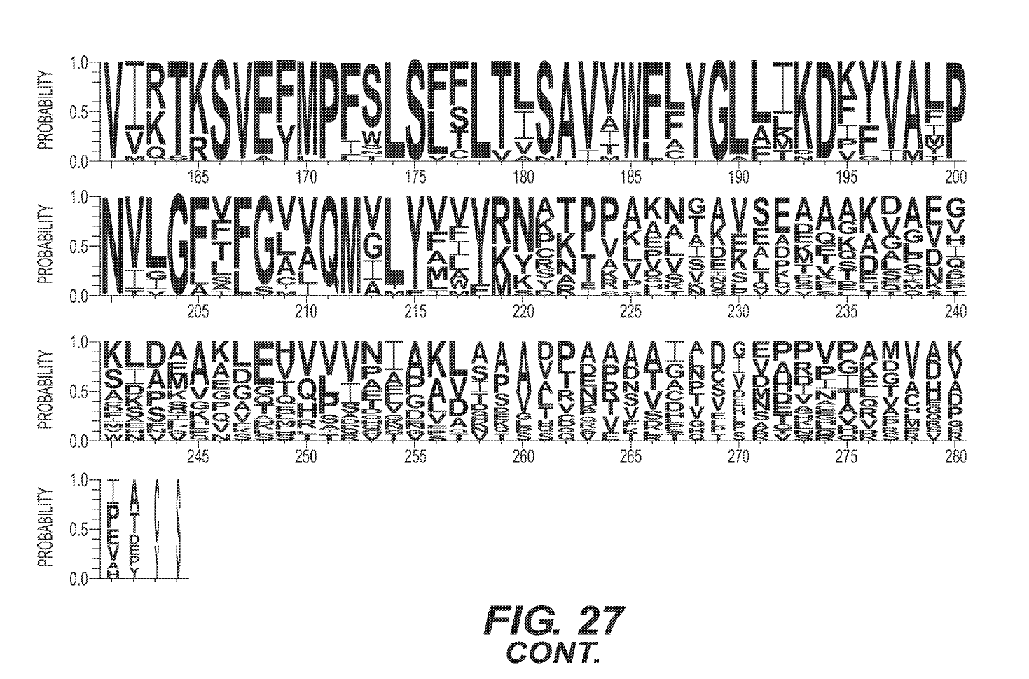

Consistent with these preliminary data that implicate SWEETs in seed filling in Arabidopsis, 12 out of the 22 SWEETs are highly expressed in maize kernels (See Maize eFP at bar.utoronto.ca/efp_maize/cgi-bin/efpWeb.cgi and QTELLER at qteller.com). Four Clade I/II hexose transporter SWEETs are highly expressed in seeds. Specifically, SWEET4b and SWEET4d are found both in embryo and endosperm, and SWEET4a and SWEET2 are expressed throughout the seed. Moreover, 8 sucrose transporting Clade III SWEETs are also expressed during seed maturation. Specifically, SWEET11, SWEET13b, SWEET13c and SWEET15b are expressed throughout the seed, SWEET14a, SWEET14b, SWEET15a and SWEET15b are expressed primarily in endosperm. Particularly, the three hexose transporters 4a,b and d (FIG. 7) in the BETL likely play crucial roles in endosperm filling. Similarly, an insertion in ZmSWEET4d obtained from UniformMu resources shows striking EMP (empty pericarp) kernel phenotype (FIGS. 8 A,B).

In the W22 background, caryopses collapse, endosperm is greatly reduced and embryo size appears smaller. This smaller phenotype is reminiscent of the documented mnl phenotype. In view of the high expression of ZmSWEET4d in BETL, the likely cause for this smaller phenotype is a block in sugar uptake into BETL affecting downstream kernel filling. The current model implicates a minimal number of transport steps, however, multiple SWEET and SUT paralogs in both Arabidopsis and maize seeds were identified, indicating greater complexity in seed filling, with the possibility of additional transport steps.

Automating the extraction, curation and reduction of spectroscopic data has greatly accelerated analysis of ¹³C-labeling data. The use of a single ¹³C-labeled sample analysis of endosperm tissue from single kernels can discriminate among four individual sibling plants from the same generation of a non-transgenic maize line. The plants and cultured kernels were grown together and the seed weights and compositions (starch, protein, oil, or cell wall contents and major soluble metabolite levels) were not significantly different among seeds from each plant. The small metabolic flux differences revealed by ¹³C-labeling patterns are due to segregation of the non-transgenic genetic background. Differences in flux profiles among 4 transgenic lines with the same growth and composition have been noted. In silico simulations using steady state flux maps are also able to predict the labeling patterns accompanying modest changes in core metabolism. A 10% change in TCA cycle flux results in ˜1% change in total carbon allocation and is associated with a distinct labeling phenotype, which can be discriminated from wild-type and other altered metabolic flux patterns, each of which yields its own label signatures or fingerprints.

SWEETs Involvement in Nectar Production

Plants have evolved anatomical and physiological features to attract animals to promote pollination. Reproductive isolation as one mechanism for speciation, is thought to be enhanced in animal pollinated species relative to wind transfer of pollen. Floral traits, including animal pollination, floral nectar spurs, bilateral symmetry and dioecious sexual system, can alter subsequent species abundance within clades. When Gaston de Saporta, Joseph Hooker, Oswald Heer and Charles Darwin discussed the ‘abominable mystery’—the apparent rapid radiation of angiosperms and insects in the mid-Cretaceous—de Saporta suggested that the development and refinement of insect-assisted pollination through the coevolution of pollinators and flowering plants may have been key to pollinator and angiosperm diversification. However the molecular mechanism of nectar secretion has remained elusive.

Flowering plants have evolved intricate methods to secure efficient interaction with pollinators and, thereby, both successful reproduction and genetic diversity through cross-pollination. Central to this process is the nectar, which contains high amounts of sugars and volatile compounds that attract and reward pollinators as well as toxins that repel unwanted floral visitors and compel pollinators to optimize outcrossing rates. Nectar composition varies widely quantitatively and qualitatively between species, presumably because it is produced to reward different families of animals. Depending on the species, 8 to 80% (w/w) of nectar is comprised of sugars, the most prevalent of which are sucrose, glucose and fructose. Nectar differs in composition from phloem sap, which delivers sugars to nectaries and is dominated by the di- and tri-saccharides sucrose and raffinose. Angiosperm nectar is synthesized and secreted by specialized organs called nectaries. Plants invest significant amounts of energy into the formation of flowers, the production of nectaries, and the secretion of sugary nectar. For example, Nicotiana attenuata, a self-compatible, hawkmoth- and hummingbird-pollinated Asterid, produces nectar that contains sucrose, hexoses and numerous secondary metabolites including nicotine. Brassica rapa, comprising self-compatible and incompatible varieties, produces hexose-dominant sugar. Arabidopsis thaliana, a self-compatible, self-fertilizer, also develops functional nectaries that produce volatiles and secrete hexose-rich nectar. It remains unclear whether nectar production in self-fertilizing plants represents an evolutionary remnant or may function to secure the low rate of outcrossing. Thus, understanding the phylogeny and biochemistry of nectar secretion may help to elucidate the processes underlying diversification of angiosperms.

Despite the importance of nectar, its secretion process has remained a matter of debate, with few functional data on the transport mechanism. To identify a transporter responsible for nectar secretion, databases of candidate sugar transporters were searched for those transporters specifically expressed in nectaries with characteristics compatible with cellular sugar efflux. Members of the recently identified SWEET sugar transporter family appeared as prime candidates for a role in nectar secretion. SWEET11 and 12 sucrose transporters are known to be responsible for cellular efflux that is key to phloem loading and, therefore, for translocation of sucrose from photosynthetic tissue to heterotrophic tissue, such as roots, flowers and seeds. Previous studies had described a SWEET9 homolog in Petunia hybrida, PhNEC1, to be specifically expressed in nectaries, and developmental timing of PhNEC1 expression has previously been correlated inversely with nectar starch content, making this transporter a prime candidate for having a role in nectar secretion. SWEET9, a close relative of NEC1, is highly expressed in Arabidopsis nectaries.

Previous studies had identified a subfamily of SWEETs as a novel class of sucrose efflux transporters responsible for moving sucrose from phloem parenchyma, the first step of loading sucrose into the vascular conduits of the phloem. Microarray and RT-PCR analyses show that SWEET9, which shares ˜50% sequence identity with SWEET11 and 12, is specifically expressed in Arabidopsis nectaries. SWEET9 is the only SWEET highly expressed in nectaries, therefore it is conceivable that SWEET9 mediates sucrose or hexose transport for nectar production. Transport studies show that SWEET9 mediates uptake and efflux of sucrose as assayed in Xenopus oocytes. Sucrose transport activity of SWEET9 was further confirmed by coexpression of SWEET9 with a Förster Resonance Energy Transfer (FRET) sucrose sensor in human embryonic kidney cells. Together these results show that SWEET9 can mediate both uptake and efflux of sucrose, consistent with the facilitated diffusion mechanism of a sucrose uniporter. Some SWEET homologs had been shown to transport both sucrose and glucose, and although the present inventors have not been able to obtain conclusive data that exclude the possibility that SWEET9 also transports hexoses, hence SWEET9 may also function in hexose efflux.

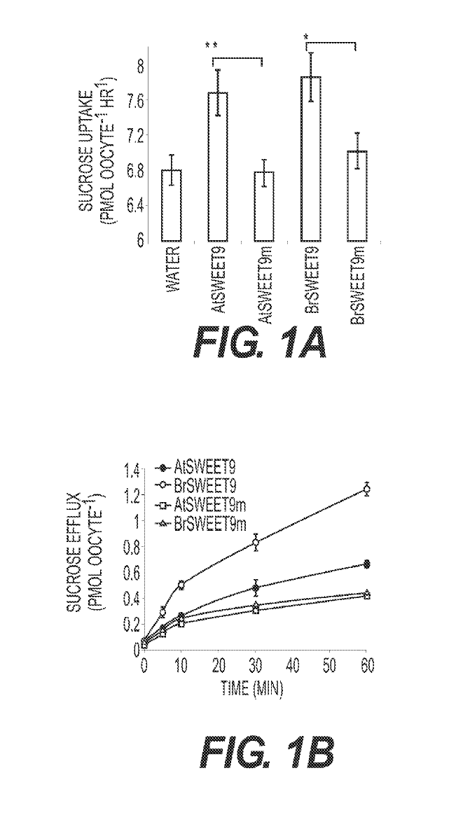

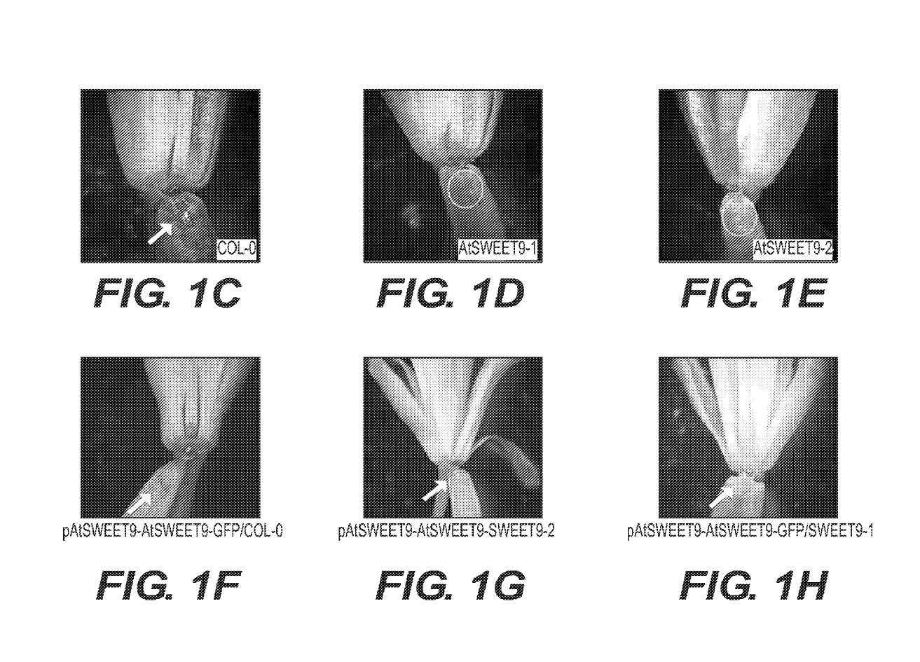

To determine directly whether SWEET9 is involved in sugar secretion from nectaries, nectar secretion was examined in three independent T-DNA insertion mutant lines [atsweet9-1, sk225 (carries a T-DNA insertion in position −308 before start codon and had no detectable transcript levels), atsweet9-2, SALK_060256 (pos. −940 before start codon and had reduced transcript levels), atsweet9-3, SALK_202913C (pos. 779 after the start codon in exon 4 or +345 from the start codon in the cDNA, knockout line). If SWEET9 plays a role in sugar uptake into or efflux from nectaries, one may expect specific phenotypes in the mutants such as but not limited to reduced sugar content, or, if the sugar efflux creates the osmotic driving force for nectar secretion, the loss of fluid secretion. In Arabidopsis, nectar droplets accumulate inside the cups formed by sepals that surround the lateral nectaries of wild-type flowers. None of the sweet9 mutants produced detectable nectar droplets (FIG. 1c-1e). Otherwise, the mutants were indistinguishable from wild-type. As judged by scanning electron microcopy (SEM), mutant nectaries had a similar morphology as wild-type nectaries, indicating that the loss of nectar secretion was not caused by a physical defect of nectaries but by loss of sugar efflux activity.

To test if SWEET9 activity is limiting for nectar secretion, nectar secretion was analyzed in transgenic lines expressing SWEET9-GFP fusions under its native promoter in wild-type background. The extra copies of SWEET9 in wild-type background showed increased nectar volume as judged by droplet size quantification (FIG. 1f). Restoration of nectar secretion by SWEET9 or SWEET9-GFP in sweet9 mutants further supports the role of SWEET9 in nectar secretion (FIGS. 1g and 1h).

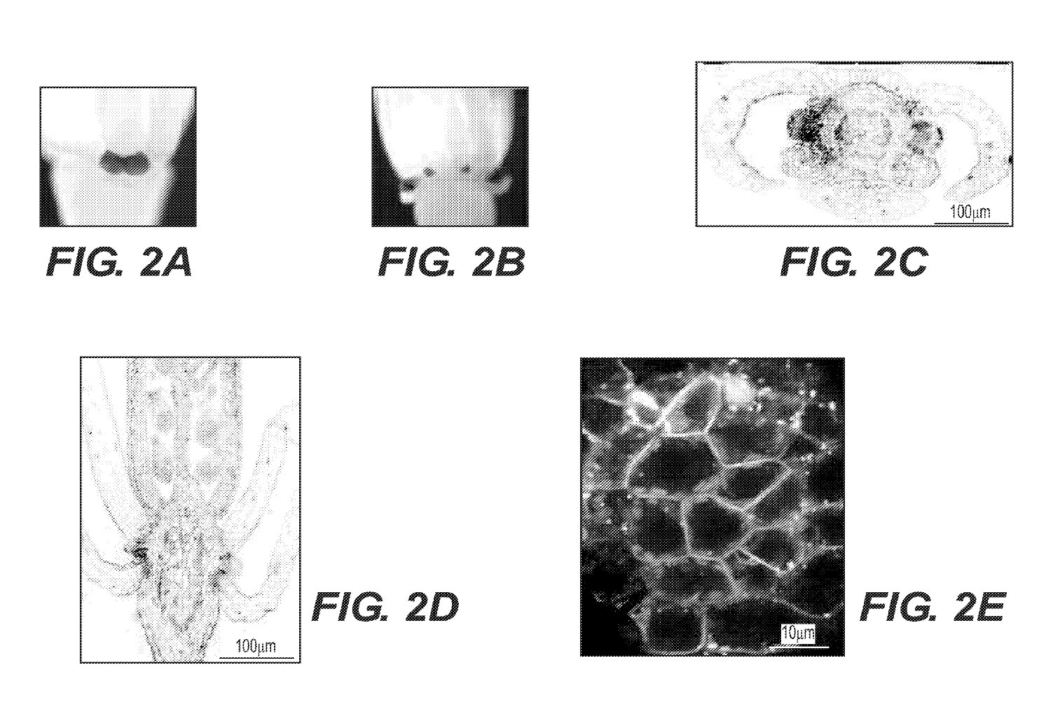

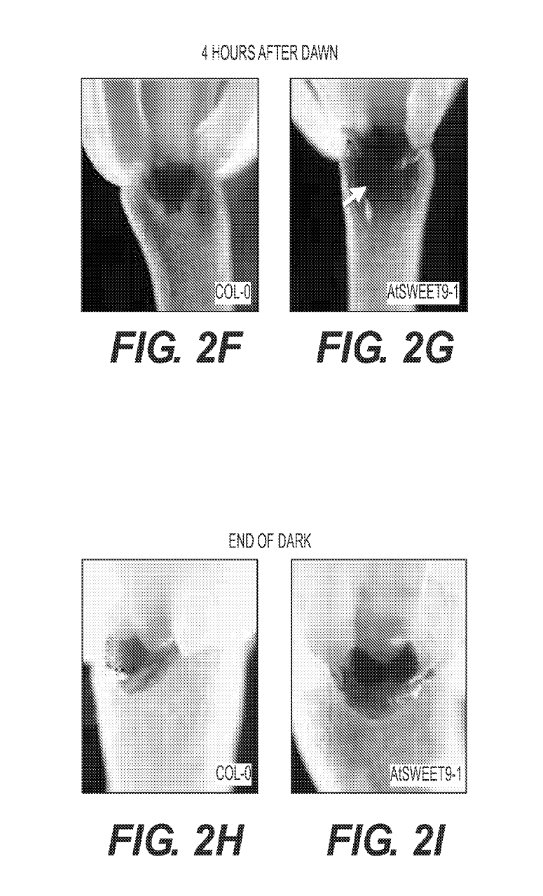

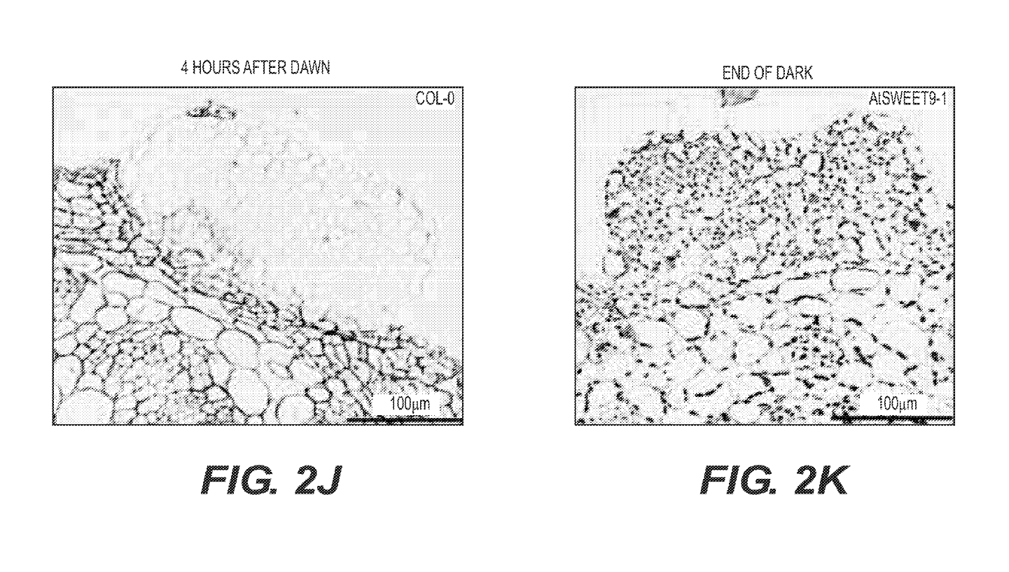

Without being bound to theory, it is possible that SWEET9 could function in at least one of at least three ways, depending on its localization. First SWEET9 may facilitate sucrose efflux at the phloem strands near nectaries, it may facilitate sugar uptake into nectary parenchyma, and/or it may facilitate sugar efflux from nectary parenchyma delivering sugars to the nectarial apoplasm. Translational fusions with GUS and eGFP under control of the SWEET9 promoter were specifically expressed in floral nectaries (FIG. 2). The highest expression was observed in the lower half of the nectary parenchyma, but not in the guard cells and phloem (FIG. 2c-d). The fluorescence intensity of SWEET9-eGFP increased during maturation, and was highest when flowers opened and when maximal nectar secretion occurs. The pattern of starch accumulation in nectaries of sweet9 mutants might be different if SWEET9 were involved in uptake into nectarial parenchyma (no starch accumulation in nectary) versus cellular efflux from nectarial cells (accumulation in nectary due to inability to export sugars). In wild type plants, starch accumulates within chloroplasts of nectary parenchyma cells before anthesis and is degraded at anthesis, serving as a source for sugar secretion.

To assess starch accumulation in sweet9-1, starch was stained with Lugol's iodine solution in fixed sections (FIG. 2f-2k). In the mutants, starch accumulated in all cells of the floral parenchyma, indicating that SWEET9 is responsible for cellular sugar efflux. Nectarial guard cells of wild-type plants contained starch granules at anthesis, but not in the sweet9 mutants. The accumulation of starch in the guard cells in wild-type nectaries may be caused by reabsorption of nectar. Taken together, the functional characterization of SWEET9 as a sucrose efflux transport, the presence of the protein in the nectary parenchyma and the pattern of starch accumulation in sweet9 mutants unable to secrete nectar, demonstrates that SWEET9 is a key transporter responsible for cellular export of sugar. High cytosolic levels of sugars in the nectarial parenchyma and extracellular hydrolysis of sucrose by a cell wall invertase create would thus facilitate the driving force for nectar secretion via this facilitated-diffusion carrier. Indeed, multiple genes in the pathway for sucrose biosynthesis were previously found to be upregulated in mature, secretory nectaries. Further, Arabidopsis nectar is a hexose:sucrose ratio of 33:1.

Since SWEET11 and 12 are plasma membrane-localized sucrose efflux transporters, we analyzed the subcellular localization of the SWEET9-promoter driven SWEET9-eGFP fusion. SWEET9-eGFP fusions localized both at the plasma membrane and the Golgi-like compartments (FIG. 2e). SWEET9 could thus operate through exocytosis or direct plasma membrane-mediated efflux. To explore any contribution of the plasma membrane localization of SWEET9 to secretion, plasma membrane localized paralogs SWEET11 and 12 were tested for their ability to restore nectar secretion in atweet9 mutants. When expressed under control of the SWEET9 promoter, both plasma membrane transporters were able to restore nectar secretion. Together, these data indicate that the impaired ability of nectar secretion of the sweet9 mutants is mainly due to reduced sugar transport across the plasma membrane. SWEET9, however, may also play a role in vesicular secretion. Except for the plasma membrane localization, SWEET9-eGFP protein also accumulated in highly mobile particles, which may be components of the Golgi or trans-Golgi network apparatus (FIG. 2e). The accumulated protein in the Golgi and Golgi-like compartments appears to serve as a reserve. Thus it is possible that SWEET9 also imports sugar into the Golgi prior to vesicular secretion.

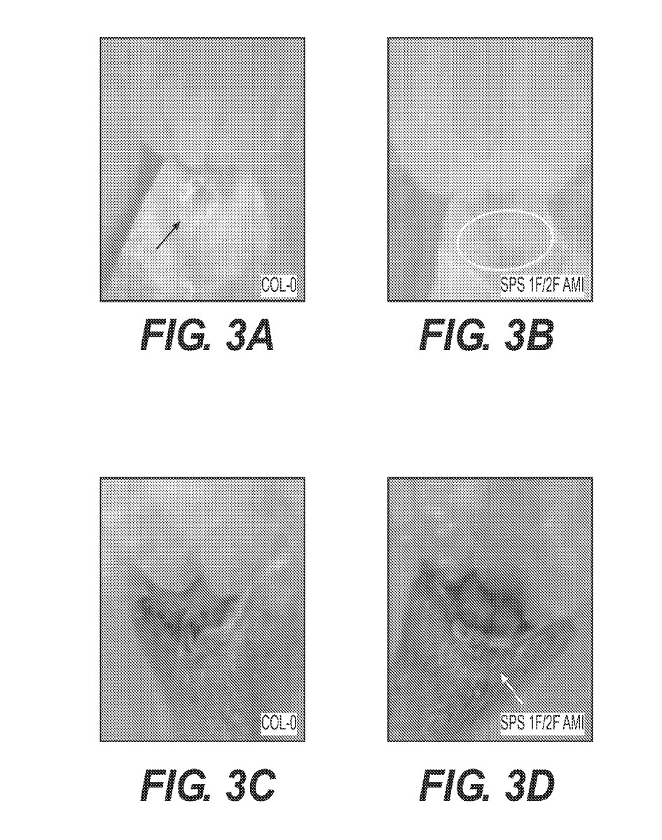

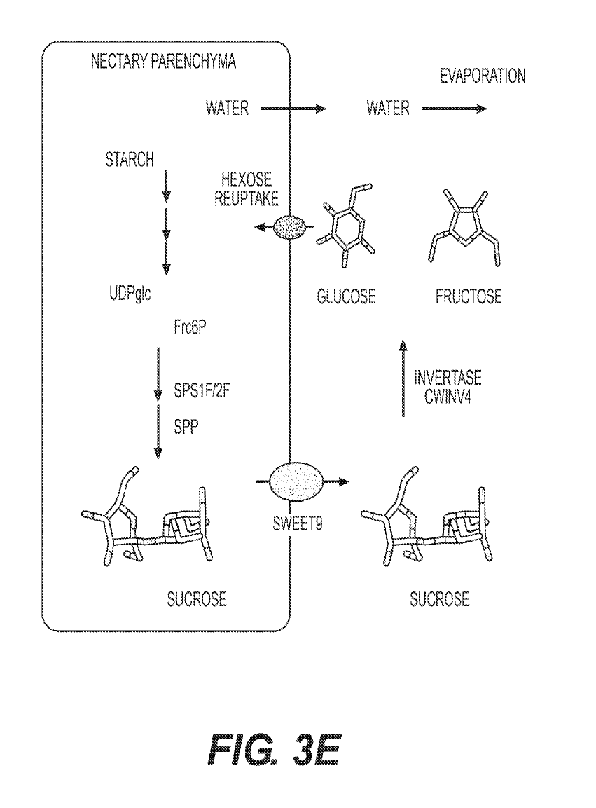

Bioinformatic analysis from previous studies of gene expression in nectaries of Arabidopsis suggests that genes involved in sucrose biosynthesis are upregulated in nectaries, indicating that resynthesis of sucrose from starch drives sugar efflux via SWEET9. The data previous suggests that two sucrose phosphate synthase (SPS) genes, both of which encode key enzymes for sucrose biosynthesis, are induced to high levels in maturing nectaries. Indeed, the SPS1F and SPS2F genes are highly expressed in nectaries. Artificial microRNA inhibition of the expression of the two SPS genes leads to a loss of nectar secretion and altered starch accumulation, which mimics the phenotype of the sweet9 mutants (FIG. 3). The phenotype of the sweet9 mutants and the SPS-miRNA lines is also similar to that of the nectarial cell wall invertase mutant cwinv4-1 that has been published previously. Together these data demonstrate that starch-derived sucrose that synthesized in nectaries is exported by SWEET9, and that sucrose hydrolysis by CWINV4 is necessary to create a sufficient osmotic gradient to sustain water secretion (FIG. 3e).

To explore whether SWEET9 is also essential for sugar efflux from nectaries of other Brassicaceae, the ortholog of SWEET9 was identified in turnip flowers (Brassica rapa). FIGS. 1a and 1b show that BrSWEET9 also transports sucrose. BrSWEET9 has previously been identified as a nectary-expressed gene. BrSWEET9, however, is also essential for sugar efflux and nectar secretion (FIGS. 1a-b and 4a-c). B. rapa belongs to the same order as Arabidopsis within the Rosid clade, and varieties can be categorized as self-incompatible outcrossers or as a self-compatible self-fertilizers. Nectar from the Rosids A. thaliana and B. rapa is predominantly composed of hexoses, which is consistent with the role of cell wall invertase in post-secretory sucrose hydrolysis.

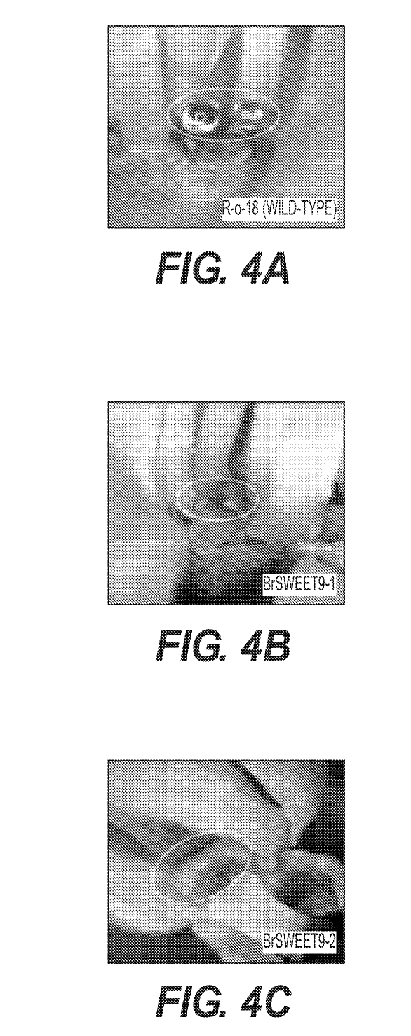

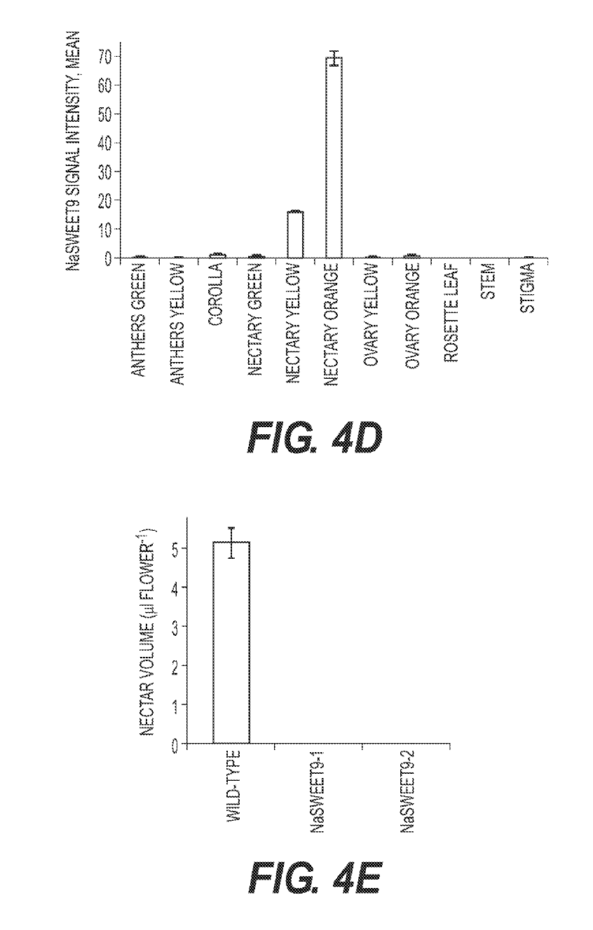

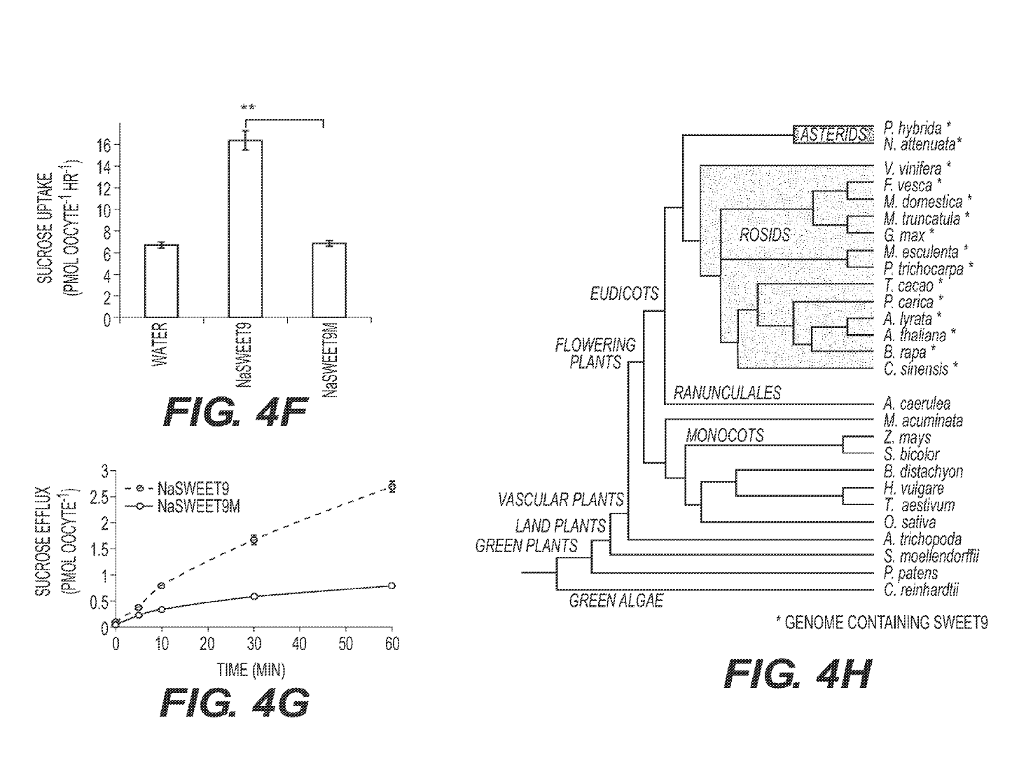

To test whether Asterids also use SWEET9 orthologs for nectar secretion, SWEET9 was identified in Nicotiana attenuata. NaSWEET9 was most highly expressed in nectaries, and expression was found to increase during nectary maturation (FIG. 4d). SWEET9 in N. attenuata mediated sucrose uptake and efflux when expressed in oocytes (FIG. 4f-g). Similar to SWEET9 in Brassicaceae, SWEET9 in N. attenuata was also essential for nectar secretion as shown in two independent RNAi lines (FIG. 4e). Together, thus SWEET9 also serves as a sugar efflux transporter at the plasma membrane of the nectary parenchyma and is necessary for secretion of nectar in core Eudicots.

A phylogenetic analysis tentatively traces the origin of SWEET9 to a point before the split of Eudicots (Asterids and Rosids; FIG. 4h) ˜120 mya. All genomes that were analyzed, including grasses, Selaginella and Physcomitrella, contain multiple SWEET paralogs. Evolution of SWEET9 may have occurred at the time when core Eudicots evolved. The presence of floral nectaries is also correlated with the existence of SWEET9. Wind-pollinated rice and maize (monocots), ancestral angiosperms such as Amborella, and basal eudicots such as Aquilegia do not appear to have SWEET9 orthologs. It is possible that, within a population, plants that differentiated a member of the SWEET Clade III into SWEET9 had a selective advantage in hijacking phloem sap from nearby sieve elements to create a secretion that would attract pollinators and thus achieve the greatest reproduction and outbreeding rates.

A model for the nectar secretion mechanism is shown in FIG. 3e. The accumulation of starch in the floral stalk of mutants may be taken as an indication that phloem-derived sucrose is imported into nectaries symplasmically. Sucrose is then hydrolyzed and stored either in the form of hexoses in the vacuole, or in the form of starch. During nectary maturation, sucrose is resynthesized via sucrose phosphate synthase, and SWEET9 begins to export sucrose down a concentration gradient, leading to sucrose accumulation in the apoplasm. Since SWEET9 appears to function as a uniporter, and since the cytosol contains other solutes that contribute to the osmotic potential, uniporter-driven efflux is unlikely to be solely sufficient for osmotically driven water secretion. Thus, sucrose in the apoplasm is then hydrolyzed by cell wall invertases to produce glucose and fructose, potentially doubling the osmotic driving force and allowing water to be secreted. Ultimately a high concentration sugary nectar is secreted through the open stomata. Together, the results presented herein show that SWEET9 serves as a sugar efflux transporter at the plasma membrane of the nectary parenchyma and is necessary for secretion of nectar in core Eudicots.

Microarray data show that the several proton-coupled sugar transporters including hexose transporting STPs are also expressed in nectaries (expressions is relatively low compared to SWEET9), indicating that these proton-coupled sugar transporters may serve as selective reuptake activities. The relative activities of cell wall invertase combined with selective reuptake activities may determine the final ratio of sucrose, fructose and glucose (FIG. 3e).

The observation of starch accumulation in mutant stems at the floral base emphasizes not only the significant energy investment involved in nectar production but also the lack of feedback regulation of sucrose delivery or translocation to other parts of the flower, even in self-pollinating plants such as Arabidopsis. That largely self-pollinating Arabidopsis has retained nectar production and produces volatiles and secretes sugary nectar to attract and reward potential pollinators suggests the importance of securing outcrossed progeny, even at a low rate. This outcrossing plays a role in coevolution and limits inbreeding depression. For highly self-pollinating species with no inbreeding depression, however, nectar sugar accumulation and sugar accumulation in floral stems of sweet9 or cwinv4 may attract pathogens and provide strong selection for reduced nectar production.

Here, the critical role of SWEET9 in nectar secretion has been shown by confirming its expression in nectaries, demonstrating its sucrose transport actions, and showing localization at both the plasma membrane and an intracellular compartment with features similar to the Golgi apparatus. Mutation of SWEET9 or nectary-expressed sucrose phosphate synthase genes led to complete loss of nectar secretion. Surprisingly, sugars delivered to defective nectaries accumulated in the stems at the floral base, indicating the lack of negative feedback on phloem delivery and the inability to relocate the sucrose efficiently. The function of SWEET9 in nectar secretion is conserved in Rosids and Asterids (the two major clades of core Eudicot species), by blocking its expression in A. thaliana, B. rapa and N. attenuata.

The Examples provided herein are meant for illustrative purposes of select embodiments of the present invention are not intended to limit the scope of the invention in any way.

### EXAMPLES

### Example 1

### Methods for Detecting SWEET9 Involvement in Nectar Production

Heterologous expression of SWEET9 from the three species in HEK293T cells and Xenopus oocytes was performed as established in the art. Insertion sites and reduced transcript levels were verified by PCR and qPCR. BrSWEET9 TILLING mutants were obtained from RevGen UK (John Innes Centre, Norwich, UK; revgenuk.jic.ac.uk/) via screening of previously described mutant populations. Wild-type N. attenuata lines were transformed by Agrobacterium tumefaciens (strain LBA 4404) to silence N. attenuata sweet9 (nasweet9). SPS1F and SPS2F were co-silenced via a single amiRNA targeting the mRNAs for both genes, and nectar secretion was evaluated using a compound microscope (Leica MZ6) by eye, and documented by photography. Starch was stained using potassium iodide. Flowers (stage 14˜15, at anthesis) were examined for starch accumulation by iodine—potassium iodide (IKI) staining (Jensen, 1962). Assay was performed following the protocol mentioned in Ruhlmann et al., 2009, which is incorporated by reference.

### Example 2

### Overexpression of Sugar Transporter SWEET9 Leads to Increased Nectar Secretion

The Arabidopsis SWEET9 gene encodes for a nectary-specific sugar transporter. Total nectar glucose content ratio and nectar droplet size of AtSWEET9 overexpression lines (driven by its native promoter) vs wild-type, were evaluated. Nectar glucose content was evaluated in each line and showed higher glucose content relative to wild-type nectar (2.04-2.66 times higher). In the same overexpressor lines, the volume of the nectar droplets was also evaluated, showing an average of 31% larger nectar volume compared with the wild-type droplets.

### Example 3

To investigate if the SWEET4d sugar transport activity within the seed BETL could be a liming factor for the sugar accumulation into the maize endosperm, transgenic A188 plants were generated to express both (i) full-length cDNA of gene GRMZM2G137954_T01 (SWEET4d) under the control of the rice Actin promoter, as well as (ii) full-length gDNA of gene GRMZM2G137954_T01 (SWEET4d) using as promoter the native 2 kb of 5′UTR upstream the ATG.

The plasmid used for the production plants containing construct (i) from above (SWEET4d overexpressors using cDNA) contained the backbone of vector pSB11 (Ishida et al., 1996), a Basta resistance cassette (rice Actin promoter and intron, Bar gene, and Nos terminator) next to the right border, and the SWEET4d coding sequence (lacking a stop codon) fused with the fluorescent protein GFP, under the control of the rice Actin promoter next to the left border. The plasmid used for the production of plants containing construct (ii) above (SWEET4d-overexpressors using genomic DNA) contained the backbone of vector pSB11 (Ishida et al., 1996), a Basta resistance cassette (rice Actin promoter and intron, Bar gene, and Nos terminator) next to the right border, and the SWEET4d full-length gDNA sequence (lacking a stop codon) fused with the fluorescent protein GFP, under the control of SWEET4d native promoter (2 kb) promoter next to the left border.

Agrobacterium-mediated transformation of maize inbred line A188 was based on a published protocol (Ishida et al., 2007). For each transformation event, the number of T-DNA insertions was evaluated by qRT-PCR, and the integrity of the transgene was verified by PCR.

References—all of which are incorporated by reference.
Kay, K. M. et al. Floral characters and species diversification. (Oxford University Press, 2006).
Harder, L. D. & Barrett, S. C. H. Ecology and evolution of flowers. (Oxford University Press, 2006).
Dodd, M. E., Silvertown, J. & Chase, M. W. Phylogenetic analysis of trait evolution and species diversity variation among angiosperm families. Evolution 53, 732-744 (1999).
Heilbuth, J. C. Lower species richness in dioecious clades. Am. Nat. 156, 221-241 (2000).
Sargent, R. D. Floral symmetry affects speciation rates in angiosperms. Proc. Biol. Sci. 271, 603-608 (2004).
Friedman, W. E. The meaning of Darwin's ‘abominable mystery’. Am. J. Bot. 96, 5-21 (2009).
De la Barrera, E. & Nobel, P. S. Nectar: properties, floral aspects, and speculations on origin. Trends Plant Sci. 9, 65-69 (2004).
Kessler, D., Gase, K. & Baldwin, I. T. Field experiments with transformed plants reveal the sense of floral scents. Science 321, 1200-1202 (2008).
Kessler, D. et al. Unpredictability of nectar nicotine promotes outcrossing by hummingbirds in Nicotiana attenuata. Plant J. 71, 529-538 (2012).
Nepi, M. & Pacini, E. Nectaries and nectar. (Springer, 2007).
Deeken, R. et al. Loss of the AKT2/3 potassium channel affects sugar loading into the phloem of Arabidopsis. Planta 216, 334-344 (2002).
Sime, K. R. & Baldwin, I. T. Opportunistic out-crossing in Nicotiana attenuata (Solanaceae), a predominantly self-fertilizing native tobacco. BMC Ecol. 3, 6 (2003).
Isokawa, S. et al. Novel self-compatible lines of Brassica rapa L. isolated from the Japanese bulk-populations. Genes Genet. Syst. 85, 87-96 (2010).
Davis, A. R., Pylatuik, J. D., Paradis, J. C. & Low, N. H. Nectar-carbohydrate production and composition vary in relation to nectary anatomy and location within individual flowers of several species of Brassicaceae. Planta 205, 305-318 (1998).
Huang, M. et al. The major volatile organic compound emitted from Arabidopsis thaliana flowers, the sesquiterpene (E)-β-caryophyllene, is a defense against a bacterial pathogen. New Phytol. 193, 997-1008 (2012).
Kram, B. W. & Carter, C. J. Arabidopsis thaliana as a model for functional nectary analysis. Sex. Plant Reprod. 22, 235-246 (2009).
Hoffmann, M. H. et al. Flower visitors in a natural population of Arabidopsis thaliana. Plant Biol. 5, 491-494 (2003).
Bomblies, K. et al. Local-scale patterns of genetic variability, outcrossing, and spatial structure in natural stands of Arabidopsis thaliana. PLoS Genet. 6, e1000890 (2010).
Nicolson, S. W., Nepi, M. & Pacini, E. Nectaries and nectar. (Springer, 2007).
Chen, L. Q. et al. Sugar transporters for intercellular exchange and nutrition of pathogens. Nature 468, 527-532 (2010).
Chen, L. Q. et al. Sucrose efflux mediated by SWEET proteins as a key step for phloem transport. Science 335, 207-211 (2012).
Ge, Y. X. et al. NEC1, a novel gene, highly expressed in nectary tissue of Petunia hybrida. Plant. 24, 725-734 (2000).
Kram, B. W., Xu, W. W. & Carter, C. J. Uncovering the Arabidopsis thaliana nectary transcriptome: investigation of differential gene expression in floral nectariferous tissues. BMC Plant Biol. 9, 92 (2009).
Ren, G. et al. Transient starch metabolism in ornamental tobacco floral nectaries regulates nectar composition and release. Plant Sci. 173, 277-290 (2007).
Langenberger, M. W. & Davis, A. R. Temporal changes in floral nectar production, reabsorption, and composition associated with dichogamy in annual caraway (Carum carvi; Apiaceae). Am. J. Bot. 89, 1588-1598 (2002).
Fahn, A. Structure and function of secretory cells. Adv. Bot. Res. 31, 37-75 (2000).
Gutierrez, R., Lindeboom, J. J., Paredez, A. R., Emons, A. M. & Ehrhardt, D. W. Arabidopsis cortical microtubules position cellulose synthase delivery to the plasma membrane and interact with cellulose synthase trafficking compartments. Nature Cell Biol. 11, 797-806 (2009).
Ruhlmann, J. M., Kram, B. W. & Carter, C. J. CELL WALL INVERTASE 4 is required for nectar production in Arabidopsis. J. Exp. Bot. 61, 395-404 (2010).
Hampton, M. et al. Identification of differential gene expression in Brassica rapa nectaries through expressed sequence tag analysis. PLoS ONE 5, e8782 (2010).
Davies, T. J. et al. Darwin's abominable mystery: Insights from a supertree of the angiosperms. Proc. Natl. Acad. Sci. USA 101, 1904-1909 (2004).
Flor, S. et al. Spatiotemporal reconstruction of the Aquilegia rapid radiation through next-generation sequencing of rapidly evolving cpDNA regions. New Phytol. 198, 579-592 (2013).
Whittall, J. B. & Hodges, S. A. Pollinator shifts drive increasingly long nectar spurs in columbine flowers. Nature 447, 706-709 (2007).
Ishida Y, et al. Agrobacterium-mediated transformation of maize. Nature Protocols 2, 1614-1621 (2007)
Ishida Y, et al. High efficiency transformation of maize (Zea mays L.) mediated by Agrobacterium tumefaciens. Nature Biotechnology 14, 745-750 (1996).
Wang et al. Control of rice grain-filling and yield by a gene with a potential signature of domestication. Nat Genet. November, 40(11):1370-4. (2008).
Wingenter K. et al. Increased activity of the vacuolar monosaccharide transporter TMT1 alters cellular sugar partitioning, sugar signaling, and seed yield in Arabidopsis. Plant Physiol. October, 154(2):665-77 (2010).

1. A method of increasing the levels of at least one sugar in developing seeds in a plant, the method comprising
a) inserting an exogenous nucleic acid in a plant cell, wherein the exogenous nucleic acid comprises a polynucleotide sequence that codes for at least one sugar transporter protein (SWEET protein), to generate a transgenic plant cell, and
b) subjecting the transgenic plant cell to conditions that promote expression of the at least one SWEET protein during seed development,
wherein the levels of at least one sugar are increased in developing seeds in the transgenic plant as compared to seeds in non-transgenic plants of the same species grown under the same conditions as the transgenic plants.

2. The method of claim 1, wherein the exogenous nucleic acid comprises at least one promoter selected from the group consisting of, a constitutive promoter operably linked to the at least one SWEET protein, a tissue-specific promoter operably linked to the at least one SWEET protein, an inducible promoter operably linked to the at least one SWEET protein.

3. The method of claim 1, wherein the at least one SWEET protein is selected from the group consisting of SWEET 1, SWEET 2, SWEET 4, SWEET 5, SWEET 7, SWEET 8, SWEET 9, SWEET 10, SWEET 11, SWEET 12, SWEET 15.

4. The method of claim 1, wherein the at least one SWEET protein is selected from the group consisting of SWEET 2, SWEET 4a, SWEET 4b, SWEET 4d, SWEET 13b, SWEET 13c, SWEET 14a, SWEET 14b, SWEET 15a, SWEET 15b.

5. The method of claim 3, wherein the SWEET protein is from the same genus or the same species of plant as the transgenic plant.

6. The method of claim 1, wherein the sugar transporter is a sucrose uniporter and the at least one sugar is sucrose, wherein the sugar transporter is a glucose uniporter and the at least one sugar is glucose, or wherein the sugar transporter is a fructose uniporter and the at least one sugar is fructose.

7. The method of claim 1, wherein the plant cell is comprised within a mature plant.

8. The method of claim 1, wherein subjecting the transgenic plant cell to conditions that promote expression of the at least one SWEET protein during seed development occurs during or after the plant cell develops into a mature plant.

9. A transgenic plant seed with increased levels of at least one sugar as compared to non-transgenic plant seeds of the same species, produced by the method of claim 1.

10. A method of making a transgenic plant that produces seeds that have increased levels of at least one sugar contained therein as compared to non-transgenic plants of the same species grown under the same conditions, the method comprising
a) inserting an exogenous nucleic acid into a plant cell, wherein the exogenous nucleic acid comprises a polynucleotide sequence that codes for at least one sugar transporter protein (SWEET protein), to generate a transgenic plant cell, and
b) growing the transgenic plant cell into a mature transgenic plant under conditions that promote expression of the at least one SWEET protein during seed development, wherein the levels of the at least one sugar are increased in developing seeds in the transgenic plant as compared to seeds from non-transgenic plants of the same species grown under the same conditions as the transgenic plants.

11. The method of claim 10, wherein the exogenous nucleic acid comprises at least one promoter selected from the group consisting of, a constitutive promoter operably linked to the at least one SWEET protein, a tissue-specific promoter operably linked to the at least one SWEET protein, an inducible promoter operably linked to the at least one SWEET protein.

12. The method of claim 10, wherein the at least one SWEET protein is selected from the group consisting of SWEET 1, SWEET 2, SWEET 4, SWEET 5, SWEET 7, SWEET 8, SWEET 9, SWEET 10, SWEET 11, SWEET 12, SWEET 15.

13. The method of claim 10, wherein the at least one SWEET protein is selected from the group consisting of SWEET 2, SWEET 4a, SWEET 4b, SWEET 4d, SWEET 11, SWEET 13b, SWEET 13c, SWEET 14a, SWEET 14b, SWEET 15a, SWEET 15b.

14. The method of claim 10, wherein the SWEET protein is from the same genus or the same species of plant as the transgenic plant.

15. The method of claim 10, wherein the sugar transporter is a sucrose uniporter and the at least one sugar is sucrose, wherein the sugar transporter is a glucose uniporter and the at least one sugar is glucose, or wherein the sugar transporter is a fructose uniporter and the at least one sugar is fructose.

16. Transgenic plant seed produced by harvesting the seeds produced in the transgenic plant that is produced by the method of claim 10.
